(19) United States
(12) Patent Application Publication
(10) Pub. No.: US 2008/0269163 A1
(43) Pub. Date: Oct. 30, 2008

(54) Methods and Compositions for Protecting Cells from Ultrasound-Mediated Cytolysis

(76) Inventors: Joe Z. Sostaric, Rockville, MD (US); Norio Miyoshi, Fukui (JP); Peter Riesz, Bethesda, MD (US)

Correspondence Address: NATIONAL INSTITUTE OF HEALTH;C/O Ballard Spahr Andrews & Ingersoll, LLP, SUITE 1000, 999 PEACHTREE STREET, ATLANTA, GA, 30309, US

(21) Appl. No.: 11/577,555

(22) PCT Filed: Oct. 19, 2005
(86) PCT No.: PCT/US2005/037912

§ 371 Date: Jun. 28, 2008

Related U.S. Application Data
(60) Provisional application No. 60/620,258, filed on Oct. 19, 2004.

(51) Int. Cl. A61K 31/70; A61K 31/7004; A61P 35/00; C12N 5/00; A61K 31/7016; A61N 7/00; A61K 31/715; A61K 31/727; A61K 31/717; A61K 31/726

(52) U.S. Cl. 514/53; 514/23; 435/375; 514/54; 514/56; 514/57; 601/2

(57) ABSTRACT

Described herein are methods for protecting cells from ultrasound-mediated cytolysis.

### CROSS-REFERENCE TO RELATED APPLICATIONS

This application claims the benefit of U.S. Provisional Application No. 60/620,258, filed Oct. 19, 2004, by Sostaric et al, which is herein incorporated by reference in its entirety.

### FIELD OF THE INVENTION

Disclosed herein are surfactants and compositions thereof that are able to protect cells from ultrasound-mediated cytolysis. Also disclosed are methods in which the disclosed surfactants and compositions thereof are delivered to cells, or cells within a subject, prior to or concurrent with the administration of ultrasound.

### BACKGROUND OF THE INVENTION

The use of ultrasound in diagnostic applications is well-known. Therapeutic uses of ultrasound, for example in physiotherapy, have been used for some time. Other therapeutic uses of ultrasound are emerging such as, for example, High Intensity Focused Ultrasound (HIFU), which is being used in patients to ablate tumors. Additionally, ultrasound energy is being used or investigated for use in gene therapy, sonoporation, transdermal drug delivery, sonodynamic therapy, cardiovascular applications, and many others. These therapeutic uses of ultrasound induce changes in tissue state, including cytolysis, through thermal effects (e.g., hyperthermia), mechanical effects (e.g., acoustic cavitation or through radiation force, acoustic streaming and other ultrasound induced forces), and chemical effects (via sonochemistry or by the activation of solutes by sonoluminescence).

Acoustic cavitation involves the formation, growth, and in certain circumstances the almost adiabatic collapse of microbubbles in a liquid medium (Neppiras, E. A. 1980; Apfel, R. E. J. 1981; Leighton, T. G. 1994). In the study of the effects of acoustic cavitation in medicine and biology, the bubbles are classified as either inertial or gas body activation (stable cavitation) bubbles (Miller, M. W. et al. 1996). When inertial bubbles collapse high temperature and pressure hot spots are formed (Noltingk, B. E. and Neppiras, E. A. 1950; Neppiras, E. A. and Noltingk, B. E. 1951; Suslick, K. S. et al. 1986; Suslick, K. S. 1990; Didenko, Y. T. et al. 1999a) which are the source of sonoluminescence (Harvey, E. N. 1939; Didenko, Y. T. et al. 1996; Didenko, Y. T. et al. 1999b; McNamara, W. B. et al. 1999) and sonochemistry (Suslick, K. S. 1988; Mason, T. J. and Lorimer, J. P. 1988; Mason, T. J. 1990). Stable bubbles oscillate around an equilibrium radius for hundreds of acoustic cycles, during which time they create regions of shear stress in their surrounding environment. This definition of bubbles is slightly ambiguous in that stable cavitation bubbles may also grow through a process of rectified diffusion to a size where they can undergo inertially driven collapse (Leighton, T G, The Acoustic Bubble; Academic Press: London, 1994, see p 335 and p 427, incorporated herein by reference for its teaching of the types of bubbles that can be found during sonolysis).

The major chemical products of inertial cavitation in a biological system have been summarized (see Miyoshi, N. et al. 2003), incorporated herein by reference for the teaching of these products. In essence, the violent collapse of inertial cavitation bubbles in an environment possessing water results in the homolysis of water vapor in the bubble to create H. atoms and .OH radicals, which are known as the primary radicals of sonolysis. The primary radicals can recombine to produce H₂O, H₂ and H₂O₂. In the presences of air, H. atoms can also react with oxygen to form the hydroperoxyl radical (HO₂.) which mostly dissociates at natural pH to the superoxide radical anion (O₂.⁻). Furthermore, the primary radicals are extremely reactive and will abstract hydrogen atoms from non-volatile organic solutes (RH), especially those that are in relatively high concentrations at the gas/solution interface of inertial cavitation bubbles. This creates carbon-centered radicals (R.) that also react with oxygen to produce relatively long lived organic peroxyl radicals (RO₂.) and other reactive oxygen radicals derived from the organic solute. The mechanism of cavitation induced cytolysis has not been fully elucidated; however cavitation bubbles could induce cytolysis through the formation of cytotoxic species such as H₂O₂ (Henglein, A. 1987) and free radical intermediates (Lippitt, B. et al. 1972; Misik, V. and Riesz, P. 1999) (inertial bubbles) and/or physical forces on the cell membrane, such as shear stress induced by acoustic streaming flow around a cavitation bubble (Neppiras, E. A. 1980; Leighton, T. G. 1994; Miller, M. W. et al. 1996; Young, F. R. 1989; Kondo, T. et al. 1989) (inertial and stable bubbles). Recently, it has been shown that even the shear forces created by a single, linearly oscillating microbubble are of large enough magnitude to cause the poration and lysis of lipid vesicles (Marmottant, P. and Hilgenfeldt, S. 2003).

Certain molecules such as, for example, thiol-based molecules (Fahey, R. C. 1988; Zheng, S. X. et al. 1988; Mitchell, J. B. et al. 1991; Aguilera, J. A et al. 1992) and nitroxides (Hahn, S. M et al. 1992a; Hahn, S. M. et al. 1992b; Newton, G. L et al. 1996) scavenge radicals in the vicinity of the nucleus of the cell and can protect against the damaging effects of ionizing radiation on mammalian cells. However, it would be difficult to envisage molecules that could protect against cavitation induced damage, which may include damage to the lipid membrane and its constituents (Ellwart, J. W. et al. 1988; Hristov, P. K. et al. 1997; Kawai, N. et al. 2003), DNA damage (Dooley, D. A. et al. 1984; Miller, D. L. et al. 1991), loss of reproductive viability (Fu, Y.-K. et al. 1979; Kondo, T. et al. 1988; Inoue, M. et al. 1989), apoptosis (Lagneaux, L. et al. 2002), and immediate cell lysis (Miyoshi, N. et al. 2003 Sacks, P. G. et al. 1982; Church, C. C. et al. 1982). The protecting molecules would presumably possess the ability to protect cells against both the chemical and physical effects of cavitation.

The beneficial effects of ultrasound in biological systems and in medicine are generally paralleled by, and are therefore limited by, the detrimental effects of ultrasound, for example, damage to healthy tissue or cytolysis of healthy cells. Thus, in many applications it would be advantageous to administer compounds that can reduce or prevent ultrasound-mediated cytolysis. For example, the glycosaminoglycans sodium hyaluronate and sodium chondroitin sulfate have been used as the major ingredients of ophthalmic viscosurgical devices (OVDs) to protect corneal endothelial cells during phacoemulsification, i.e., the use of ultrasound to break the cataract into very minute fragments and pieces (Miyata K, et al. 2002. J Cataract Refract Surg. 28(9):1557-60). These OVDs function, in part, by forming a meshwork structure that adheres to the endothelial cells during phacoemulsification. Although the mechanism of protection is not known, it has been suggested that this OVD mesh protects the endothelial cells from the detrimental effects of ultrasound-induced radicals, due to their antioxidant properties (Takahashi H, et al. 2002. Arch Opthalmol. 120(10):1348-52).

However, the high viscosity of the long chain glycosaminoglycans, will also alter the viscosity of the system, which interferes with the formation and dynamics of acoustic cavitation bubbles and thus the potentially positive effects of ultrasound. In other systems, for example in a suspension of cells in vitro or in tissue deep inside the human body, it would be impractical, if not impossible, to create such a viscous framework on the cells or on the tissue, respectively. Instead of applying a viscous mesh-like structure on the surface of cells, it would be advantageous to provide molecules that protect cells from ultrasound mediated cytolysis with only a minimal to no effect on the physical properties induced by ultrasound. It would be further advantageous to use relatively small solute molecules that can accumulate at the gas/solution interface of acoustic cavitation bubbles and protect cells from ultrasound mediated damage at the site of radical formation. The compounds, compositions, and methods described herein accomplish this goal over a broad range of ultrasound frequency and intensity conditions, and create new opportunities for the use of ultrasound in diagnostic, therapeutic and biotechnological applications not currently available.

### SUMMARY OF THE INVENTION

Described herein are methods for protecting cells from ultrasound-mediated cytolysis. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows particle size distribution of HL-60 cells measured with the Coulter counter (a) healthy, untreated HL-60 cells. The effect of sonolysis of HL60 cells at 1.057 MHz for 15 seconds and power=30 W is shown for (b) no additives and (c) in the presence of 5 mM HGP. *The graphs shown are edited versions of photographs taken from the monitor readout of the Coulter counter instrument. The original photographs were slightly rotated, cropped and the color adjusted to produce FIG. 1. Therefore, the values of the x-axis and also the relative heights of the particle distributions for a, b and c are not exact, however they are a very close approximation of the originals.

FIG. 2 shows the effect of various glucopyranosides on the percentage cytolysis observed as a function of glucopyranoside concentration (0-10 mM), following Coulter counter analysis: ◯ MGP; ▪ HGP; Δ HepGP; ♦ OGP. The insert shows this effect in the glucopyranoside concentration range of 0-30 mM. Conditions: 1.057 MHz ultrasound, air exposed, 298 K, power=10 W, sonolysis time=5 s, % cytolysis ±SD (n=5 to 8).

FIG. 3 shows Coulter counter analysis of HL-60 cells (1 ml suspensions) exposed to ultrasound (1.057 MHz) at various powers and ultrasound exposure times in the presence of HGP (5 mM). % cytolysis ±SD (n=5 to 8).

FIG. 4 shows reproduction assay following sonolysis at 1.057 kHz, time=5 seconds, p=10 W and various glucopyranosides. Cells were allowed to reproduce for 24 hours. Reproduction fraction ±SD (where n=6 to 8) is calculated by dividing the number of cells counted following 24 hours of reproduction by the number of cells counted at the start of incubation.

FIG. 5 shows reproduction assay of HL-60 cells under control (□, i.e., no sonolysis) and relatively extreme sonolysis conditions: frequency=1.057 MHz; time=15 seconds; 1 ml HL-60 cells; HGP 5 mM and power=(a) ·40 W; (b)×60 W. On the log scale shown, the error bars are within the size of the data points. Control data points were run in triplicate with a standard deviation of less than 10%. Sonolysis points represent an average of six separate cell suspensions, with a standard deviation of less than 10%.

FIG. 6 shows mechanical fragility of cells determined using a Burrell wrist action shaker set to 50% power. 10 ml borosilicate glass beads in a 125 ml conical flask. 10 ml cell suspension in the presence and absence (control) of various glucopyranosides (HGP, HepGP, MGP, and OGP) in a cell culture medium containing 10% DPBS solution. Shaking was conducted over a period of 30 minutes. Each data point represents ±SD, where n=5.

FIG. 7 shows ESR spectra observed following sonolysis of cell suspensions in the presence of DBNBS (3 mg/ml); (a) no glucopyranoside and (b) 5 mM HGP. Tertiary carbon-centered radicals are labeled 1, secondary carbon-centered radicals are labeled 2. Primary carbon-centered radicals are labeled 3. Conditions of sonolysis: frequency=1.057 MHz; time=15 seconds, power=60 W, argon-saturated solutions, temperature=25° C.

FIG. 8 shows explanation of the events occurring around inertial cavitation bubbles during sonolysis of a cell suspension (a) in RPMI 1640 medium and (b) in the presence of 2 to 5 mM concentrations of HGP, HepGP or OGP, glucopyranoside surfactants during sonolysis at 1 MHz frequency.

FIG. 9 shows the effect of various glucopyranosides on the percentage cytolysis observed as a function of glucopyranoside concentration (0-10 mM), following Coulter counter analysis: ◯ MGP; ▪ HGP; A HepGP; ♦ OGP. Conditions: 614 kHz ultrasound, air exposed, 298 K, power=20 W, sonolysis time=5 s, % cytolysis ±SD (n=5 to 8). MGP data was gathered at half the ultrasound power of the other experiments, i.e., 10 W.

FIG. 10 shows the effect of various glucopyranosides on the percentage cytolysis observed as a function of glucopyranoside concentration (0-10 mM), following Coulter counter analysis: ◯ MGP; ▪ HGP; ♦ OGP. Conditions: 354 kHz ultrasound, air exposed, 298 K, power=15 W, sonolysis time=5 s, % cytolysis ±SD (n=5 to 8).

FIG. 11 shows the effect of various glucopyranosides on the percentage cytolysis observed as a function of glucopyranoside concentration (0-10 mM), following Coulter counter analysis: ◯ MGP; ▪ HGP; ♦ OGP. Conditions: 42 kHz ultrasound, air exposed, 298 K, power=50% reduction of original, sonolysis time=5 s, % cytolysis ±SD (n=5 to 8).

FIG. 12 shows the effect of ultrasound frequency on the sonoprotecting properties of glucopyranosides. The data from FIGS. 12a to 12d has been normalized at zero glucopyranoside concentration to compare the effect of ultrasound frequency on the sonoprotecting ability of any particular glucopyranoside: (a) OGP; (b) HepGP; (c) HGP and (d) MGP.

FIG. 13 shows the effect of hexyl-β-D-maltopyranoside (HMP) on the percentage cytolysis observed as a function of HMP concentration (0-3 mM), following Coulter counter analysis. Conditions: 1.057 MHz ultrasound, air exposed, 298 K, power=10 W, sonolysis time=5 s, % cytolysis ±SD (n=4-6)

FIG. 14 shows the effect of n-octyl-β-D-maltopyranoside (OMP) on the percentage cytolysis observed as a function of OMP concentration (0-3 mM), following Coulter counter analysis. Conditions: 1.057 MHz ultrasound, air exposed, 298 K, power=10 W, sonolysis time=5 s, % cytolysis ±SD (n=6)

FIG. 15 shows the effect of n-octyl-β-D-thioglucopyranoside (OTGP) on the percentage cytolysis observed as a function of OTGP concentration (0-3 mM), following Coulter counter analysis. Conditions: 1.057 MHz ultrasound, air exposed, 298 K, power=10 W, sonolysis time=5 s, % cytolysis ±SD (n=6)

FIG. 16 shows the effect of 2-propyl-1-pentyl-β-D-maltopyranoside (PPMP) on the percentage cytolysis observed as a function of PPMP concentration (0-3 mM), following Coulter counter analysis. Conditions: 1.057 MHz ultrasound, air exposed, 298 K, power=10 W, sonolysis time=5 s, % cytolysis ±SD (n=6)

FIG. 17 shows the effect of Isopropyl-β-D-thiogalactopyranoside (IPTGalP) on the percentage cytolysis observed as a function of IPTGalP concentration (0-25 mM), following Coulter counter analysis. Conditions: 1.057 MHz ultrasound, air exposed, 298 K, power=10 W, sonolysis time=5 s, % cytolysis ±SD (n=6)

FIG. 18 shows the “reproduction ratio,” which is a measure of the ability of the surviving cell population to continue reproducing following treatment by ultrasound in the presence or absence of n-hexyl-β-D-glucopyranoside (HGP). The reproduction ratio is the number of cells present one or two days post treatment divided by the number of cells present on the treatment day.

FIG. 19 shows the effect of n-octyl-α-D-glucopyranoside (alphaOGP) on the percentage cytolysis observed as a function of alphaOGP concentration (0-3 mM), following Coulter counter analysis. Conditions: 1.057 MHz ultrasound, air exposed, 298 K, power=10 W, sonolysis time=5 s, % cytolysis ±SD (n=6).

FIG. 20 shows the effect of Methyl-6-O—(N-heptylcarbamoyl)-α-D-glucopyranoside (ANAMEG-7) on the percentage cytolysis observed as a function of ANAMEG-7 concentration (0-5 mM), following Coulter counter analysis. Conditions: 1.057 MHz ultrasound, air exposed, 298 K, power=10 W, sonolysis time=5 s, % cytolysis ±SD (n=6).

FIG. 21 shows the effect of 3-Cyclohexyl-1-propyl-β-D-glucoside (Cyglu-3) on the percentage cytolysis observed as a function of CYGLU-3 concentration (0-5 mM), following Coulter counter analysis. Conditions: 1.057 MHz ultrasound, air exposed, 298 K, power 10 W, sonolysis time=5 s, % cytolysis ±SD (n=6).

FIG. 22 shows the effect of 6-O-Methyl-n-Heptylcarboxyl-α-D-Glucopyranoside (MHC-alpha-GP) on the percentage cytolysis observed as a function of MHC-alpha-GP concentration (0-5 mM), following Coulter counter analysis. Conditions: 1.057 MHz ultrasound, air exposed, 298 K, power=10 W, sonolysis time=5 s, % cytolysis ±SD (n 6).

FIG. 23 shows the effect of glucopyranosides (MGP, HGP, OGP) on sonolysis of HL-525 cells. Conditions: 42 kHz, 50% power, 5 sec, 20° C., % cytolysis ±SD (n=6).

FIG. 24 shows the effect of glucopyranosides (MGP, HGP, HepGP, OGP) on sonolysis of HL-525 cells. Conditions: 354 kHz, 15 W, 5 sec, 20° C., % cytolysis ±SD (n=6).

FIG. 25 shows the effect of glucopyranosides (MGP, HGP, HepGP, OGP) on sonolysis of HL-525 cells. Conditions: 614 kHz, 20 W, 5 sec, 20° C., % cytolysis ±SD (n=6).

FIG. 26 shows the effect of glucopyranosides (MGP, HGP, HepGP, OGP) on sonolysis of HL-525 cells. Conditions: 1057 kHz, 10 W, 5 sec, 20° C., % cytolysis ±SD (n=6).

FIG. 27 shows the effect of different concentrations of glucopyranosides (MGP, HGP, HepGP, OGP) on mechanical fragility of HL-525 cells. Conditions: 50% power, 30 min. shaking, 10 mL borosilicate glass beads, 10 mL cell suspension.

### DETAILED DESCRIPTION

The present invention may be understood more readily by reference to the following detailed description of preferred embodiments of the invention and the Examples included therein and to the Figures and their previous and following description.

Before the present compounds, compositions, articles, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

A weight percent of a component, unless specifically stated to the contrary, is based on the total weight of the formulation or composition in which the component is included.

Variables such as R¹-R⁹, X, and Y used throughout the application are the same variables as previously defined unless stated to the contrary.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “ester” as used herein is represented by the formula —C(O)OR, where R can be an alkyl, alkenyl, alkynyl, aryl, heteroaryl, cycloalkyl, cycloalkenyl, heterocycloalkyl, or heterocycloalkenyl group described above.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, or heterocycloalkyl group described above.

The term “amide” as used herein is represented by the formula —C(O)NR, where R can be an alkyl, alkenyl, alkynyl, aryl, heteroaryl, cycloalkyl, cycloalkenyl, heterocycloalkyl, or heterocycloalkenyl group described above.

The phrase “or a combination thereof” with respect to R1-R7 referred to herein means each of R1-R7 can optionally possess two or more of the groups listed above. For example, if R1 is a straight chain alkyl group, one of the hydrogen atoms of the alkyl group can be substituted with another group such as, for example, an aryl group or cycloalkyl group. Here R1 is a combination of an alkyl group and an aryl group.

The term “monosaccharide” as used herein is any carbohydrate that cannot be broken down into simpler units by hydrolysis.

The term “disaccharide” as used herein is any carbohydrate that is produced from two monosaccharide units.

The term “polysaccharide” as used herein is any carbohydrate that is produced from more than two monosaccharide units.

The term “residue” as used herein refers to the moiety that is the resulting product of the chemical species in a particular reaction scheme or subsequent formulation or chemical product, regardless of whether the moiety is actually obtained from the chemical species. For example, a polysaccharide that contains at least one —COOH group can be represented by the formula Y—COOH, where Y is the remainder (i.e., residue) of the polysaccharide molecule.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Provided herein are compositions and methods for protecting cells from ultrasound-mediated cytolysis. In one aspect, the composition comprises a sonoprotectant (also referred to herein as sonoprotector). In a further aspect, the sonoprotectant comprises a surfactant. In a yet further aspect, the sonoprotectant comprises two or more surfactants. The term “surfactant” is used herein to designate a substance which exhibits some superficial or interfacial activity between a liquid-liquid interface or gas-liquid interface. The surfactant can be anionic, cationic, or neutral depending upon the surfactant selected, the mode of administration, and the cells to be treated. The use of amphoteric or zwitterionic surfactants (i.e., surfactant molecule exhibits both anionic and cationic properties) are also contemplated.

In one aspect, the method comprises administering to the cells a surfactant, wherein the surfactant comprises a carbohydrate comprising at least one hydrophobic group. The term “carbohydrate” is defined herein as a polyhydroxy aldehyde or ketone. The carbohydrate can be a monosaccharide, a disaccharide, or a polysaccharide as defined above. It is contemplated that the carbohydrate can be cyclic or acyclic. In the case of cyclic carbohydrates useful herein, the term “pyranoside” as used herein is the ring-form of an acyclic carbohydrate. Carbohydrates can readily be converted to the cyclic and acyclic forms using techniques known in the art. Examples of monosaccharides include, but are not limited to, 2-deoxyribose, fructose, idose, gulose, talose, galactose, mannose, altrose, allose, xylose, lyxose, arabinose, ribose, threose, glucosamine, erythrose, or the pyranoside thereof. In one aspect, the monosaccharide is a glucopyranoside. Examples of disaccharides include, but are not limited to, lactose, cellobiose, or sucrose. In one aspect, the disaccharide is a maltosepyranoside. Examples of polysaccharides include, but are not limited to, hyaluronan, chondroitin sulfate, dermatan, heparan, heparin, dermatan sulfate, and heparan sulfate, alginic acid, pectin, or carboxymethylcellulose.

In the aspect above, the surfactant is a carbohydrate having at least one hydrophobic group. The term “hydrophobic group” is defined herein as any group that has little to no affinity to water. The hydrophobic group is generally covalently attached to the carbohydrate group. It is contemplated that two or more hydrophobic groups can be attached to the carbohydrate. In one aspect, the hydrophobic group is a branched- or straight-chain alkyl group having from 1 to 25 carbon atoms. In another aspect, the hydrophobic group is a C₁-C₂₀, C₁-C₁₅, C₁-C₁₀, C₂-C₁₅, C₃-C₁₅, C₄-C₁₅, C₅-C₁₅, C₅-C₁₀, C₂-C₉, or C₄-C₉ branched- or straight-chain alkyl group.

In one aspect, described herein is a method for protecting cells from ultrasound-mediated cytolysis, comprising delivering to the cells a surfactant, wherein the surfactant comprises at least one unit having the formula I

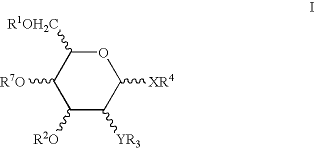

wherein X is oxygen, sulfur, or NR⁵, and

Y is oxygen, sulfur, or NR⁶,

wherein R¹-R⁷ are each, independently, hydrogen, a branched- or straight-chain alkyl group, a substituted or unsubstituted aryl group, an aralkyl group, a cycloalkyl group, an ester group, an aldehyde group, a keto group, an amide group, a residue of a saccharide, or a combination thereof,

or the pharmaceutically-acceptable salt or ester thereof,

wherein at least one of R¹-R⁷ is a hydrophobic group,

wherein the surfactant is not sodium chondroitin sulfate, sodium hyaluronate, or a combination thereof.

In another aspect, described herein is a method for protecting cells from ultrasound-mediated cytolysis, comprising delivering to the cells a surfactant, wherein the surfactant comprises at least one unit having the formula I

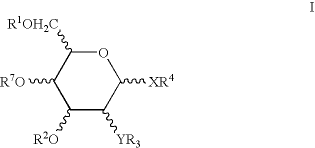

wherein X is oxygen, sulfur, or NR⁵, and

Y is oxygen, sulfur, or NR⁶,

wherein R¹-R⁷ are each, independently, hydrogen, a branched- or straight-chain alkyl group, a substituted or unsubstituted aryl group, an aralkyl group, a cycloalkyl group, an ester group, an aldehyde group, a keto group, an amide group, a residue of a saccharide, or a combination thereof,

or the pharmaceutically-acceptable salt or ester thereof,

wherein at least one of R¹-R⁷ is a hydrophobic group,

wherein the surfactant has a molecular weight of less than 5,000 Da.

In these aspects, the term “unit” with respect to the surfactant is a compound having at least one fragment having the formula I incorporated in the surfactant. For example, when the surfactant is a polysaccharide, the unit having the formula I can be incorporated within the polysaccharide chain or at the terminus of the polysaccharide chain. Referring to formula I, when R⁴ and R⁷ are a residue of a saccharide, the unit having the formula I is incorporated in the polysaccharide chain. Alternatively, when R⁴ is hydrogen and R⁷ is a residue of a saccharide, the surfactant is terminated with a unit having the formula I. The term “saccharide” is defined herein as any monosaccharide, disaccharide, or polysaccharide defined above. In one aspect, the surfactant can be a disaccharide having the unit of formula I (e.g., R⁷ is a monosaccharide). In another aspect, the surfactant is a monosaccharide of unit I, where R¹-R⁷ is not a residue of a saccharide.

It is contemplated that when the surfactant is a carbohydrate (e.g., a carbohydrate having at least one unit of the formula I), the carbohydrate can assume a number of different configurations. In one aspect, the carbohydrate can exist as an acetal or hemiacetal. Additionally, when the surfactant is a carbohydrate, different anomers and epimers are contemplated as well.

In one aspect, the molecular weight of the surfactant is less than 5,000 Da, less than 4,500 Da, less than 4,000 Da, less than 3,500 Da, less than 3,000 Da, less than 2,500 Da, less than 2,000 Da, less than 1,500 Da, less than 1,000 Da, less than 500 Da, less than 400 Da, or less than 300 Da. In another aspect, the surfactant has 10, 9, 8, 7, 6, 5, 4, 3, 2, or 1 unit having the formula I. Not wishing to be bound by theory, the surfactant is a compound that does not necessarily have to alter the viscosity of the target, i.e., the medium, plasma, or intercellular fluid of cells; the surface of cells or the surface of tissue; cells of a subject or regions within a subject that will be treated with ultrasound. Any changes in viscosity would be incidental and not a requirement for sonoprotection. Thus, in one aspect, the surfactant is a compound that does not significantly alter the viscosity of the target. In another aspect, the surfactant is not high molecular weight sodium hyaluronate or sodium chondroitin sulfate sold under the trade name HEALON® (Alcon laboratories, Inc.) or VISCOAT® (Pharmacia).

In one aspect, R⁴ is a hydrophobic group and R¹-R³ and R⁷ are, independently, hydrogen or a residue of a saccharide. In another aspect, R⁴ is a hydrophobic group, R¹-R³ are hydrogen, and R⁷ is hydrogen or a residue of a saccharide. In a further aspect, R⁷ is a hydrophobic group and R¹-R⁴ are, independently, hydrogen or a residue of a saccharide. In another aspect, R⁷ is a hydrophobic group, R¹-R³ are hydrogen, and R⁴ is hydrogen or a residue of a saccharide.

In another aspect, when the surfactant has at least one unit having the formula I, at least one of R¹-R⁴ and R⁷ is hydrogen. In another aspect, X and Y are oxygen. In any of the preceding aspects, R¹-R³ are hydrogen. In any of the preceding aspects, R⁷ is hydrogen.

In another aspect, R⁷ of unit I is a residue of a saccharide. In one aspect, the saccharide is a monosaccharide such as, for example, 2-deoxyribose, fructose, idose, gulose, talose, galactose, mannose, altrose, allose, xylose, lyxose, arabinose, ribose, threose, glucosamine, erythrose, or the pyranoside thereof. In another aspect, R⁷ of unit I is a glucopyranoside.

In another aspect, R⁴ of unit I is the hydrophobic group. In one aspect, R⁴ is a branched- or straight chain C₁-C₂₅, C₁-C₂₀, C₁-C₁₅, C₁-C₁₀, C₂-C₁₅, C₃-C₁₅, C₄-C₁₅, C₅-C₁₅, C₅-C₁₀, C₂-C₉, or C₄-C₉ alkyl group. In another aspect, R⁴ is methyl, ethyl, propyl, isopropyl, butyl, pentyl, hexyl, heptyl, octyl, nonyl, or decyl.

In another aspect, R¹ in unit I is the hydrophobic group. In one aspect, R¹ is C(O)R⁸, wherein R⁸ is a branched- or straight chain C₁-C₂₅, C₁-C₂₀, C₁-C₁₅, C₁-C₁₀, C₂-C₁₅, C₃-C₁₅, C₄-C₁₅, C₅-C₁₅, C₅-C₁₀, C₂-C₉, or C₄-C₉ alkyl group. In another aspect, R¹ is C(O)NHR⁹, wherein R⁹ is a branched- or straight chain C₁-C₂₅, C₁-C₂₀, C₁-C₁₅, C₁-C₁₀, C₂-C₁₅, C₃-C₁₅, C₄-C₁₅, C₅-C₁₅, C₅-C₁₀, C₂-C₉, or C₄-C₉ alkyl group. In either of these aspects, R², R³, and R⁷ are hydrogen. In any of the preceding aspects, R⁴ is a branched- or straight chain C₁ to C₂₅ alkyl group such as, for example, methyl, ethyl, propyl, butyl, pentyl, or hexyl.

In one aspect, the surfactant having the unit I is the α-anomer. In another aspect, the surfactant having the unit I is the α-anomer.

The surfactants useful herein can be prepared using techniques known in the art. Alternatively, surfactants that are commercially available can be used in the methods described herein. For example, the alkylated carbohydrates sold by Anatrace, Inc., Maumee, Ohio, USA can be used herein.

In one aspect, the surfactant is an alkyl-β-D-thioglucopyranoside, an alkyl-β-D-thiomaltopyranoside, alkyl-β-D-galactopyranoside, an alkyl-β-D-thiogalactopyranoside, or an alkyl-β-D-maltrioside.

Examples of alkyl-β-D-thioglucopyranosides include, but are not limited to, hexyl-β-D-thioglucopyranoside, heptyl-β-D-thioglucopyranoside, octyl-β-D-thioglucopyranoside, nonyl-β-D-thioglucopyranoside, decyl-β-D-thioglucopyranoside, undecyl-β-D-thioglucopyranoside, or dodecyl-β-D-thioglucopyranoside. Examples of alkyl-β-D-thiomaltopyranosides include, but are not limited to, octyl-β-D-thiomaltopyranoside, nonyl-β-D-thiomaltopyranoside, decyl-β-D-thiomaltopyranoside, undecyl-β-D-thiomaltopyranoside, or dodecyl-β-D-thiomaltopyranoside.

In another aspect, the surfactant is an alkyl-β-D-glucopyranoside. Examples of alkyl-β-D-glucopyranosides include, but are not limited to, hexyl-β-D-glucopyranoside, heptyl-β-D-glucopyranoside, octyl-β-D-glucopyranoside, nonyl-β-D-glucopyranoside, decyl-β-D-glucopyranoside, undecyl-β-D-glucopyranoside, dodecyl-β-D-glucopyranoside, tridecyl-β-D-glucopyranoside, tetradecyl-β-D-glucopyranoside, pentadecyl-β-D-glucopyranoside, hexadecyl-β-D-glucopyranoside, methyl-6-O—(N-heptylcarbamoyl)-α-D-glucopyranoside, 6-O-methyl-n-heptylcarboxyl)-α-D-glucopyranoside, or 3-cyclohexyl-1-propyl-β-D-glucopyranoside.

In another aspect, the surfactant is an alkyl-β-D-maltopyranoside. Examples of alkyl-β-D-maltopyranosides include, but are not limited to, 2-propyl-1-pentyl-β-D-maltopyranoside, hexyl-β-D-maltopyranoside, heptyl-β-D-maltopyranoside, octyl-β-D-maltopyranoside, nonyl-β-D-maltopyranoside, decyl-β-D-maltopyranoside, undecyl-β-D-maltopyranoside, dodecyl-β-D-maltopyranoside, tridecyl-β-D-maltopyranoside, tetradecyl-β-D-maltopyranoside, pentadecyl-β-D-maltopyranoside, or hexadecyl-β-D-maltopyranoside.

In another aspect, the surfactant is laetrile, arbutin, salicin, digitoxin, n-lauryl-beta-D-maltopyranoside, glycyrritin, p-nitrophenyl-beta-D-glucopyranoside, p-nitrophenyl-beta-D-galactopyranoside, p-nitrophenyl-beta-D-lactopyranoside, or p-nitrophenyl-beta-D-maltopyranoside.

In another aspect, the surfactant is derived from a naturally-occurring product. In one aspect, the surfactant is (Z)-5′-hydroxyjasmone 5′-O-beta-D-glucopyranoside or 3′-O-beta-D-glucopyranosyl-catalpol isolated from the aerial part of Asystasia intrusa; prinsepiol-4-O-beta-D-glucopyranoside and fraxiresinol-4′-O-beta-D-glucopyranoside isolated from the roots of Valeriana prionophylla; quercetin 3-O-alpha-L-arabinopyranosyl-(1-->2)-beta-D-glucopyranoside, kaempferol 3-O-beta-D-glucopyranoside, and quercetin 3-O-beta-D-glucopyranoside isolated from the leaves of Eucommia ulmoides; catechin (4-alpha-->8) pelargonidin 3-O-beta-glucopyranoside, epicatechin (4-alpha-->8) pelargonidin 3-O-beta-glucopyranoside, afzelechin (4-alpha-->8) pelargonidin 3-O-beta-glucopyranoside, and epiafzelechin (4-alpha-->8) pelargonidin 3-O-beta-glucopyranoside isolated from strawberries; and quercetin 3,7-O-beta-D-diglucopyranoside, quercetin 3-O-alpha-L-rhamnopyransol-(1-->6)-beta-D-glucopyranosol-7-O-beta-D-glucopyranoside, isorhamnetin-3-O-beta-D-6′-acetylglucopyranoside, and isorhamnetin-3-O-beta-D-6′-acetylgalactopyranoside extracted from Hemerocallis leaves.

Any of the surfactants described herein can be the pharmaceutically acceptable salt or ester thereof. Pharmaceutically acceptable salts are prepared by treating the free acid or alcohol with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

In another aspect, if the surfactant possesses a basic group, it can be protonated with an acid such as, for example, HCl or H₂SO₄, to produce the cationic salt. In one aspect, the reaction of the surfactant with the acid or base is conducted in water, alone or in combination with an inert, water-miscible organic solvent, at a temperature of from about 0° C. to about 100° C. such as at room temperature. In certain aspects where applicable, the molar ratio of the surfactants described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the surfactants and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

It is contemplated that the pharmaceutically-acceptable salts or esters of the surfactants described herein can be used as prodrugs or precursors to the active compound prior to the administration. For example, if the active surfactant is unstable, it can be prepared as its salts form in order to increase stability.

Any of the surfactants described herein can be formulated with a pharmaceutically acceptable carrier to produce a pharmaceutical composition. By “pharmaceutically acceptable” is meant a material that is not biologically or otherwise undesirable, i.e., the material may be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

As used throughout, administration of any of the surfactants and compositions described herein can occur in conjunction with other therapeutic agents. Thus, the surfactant can be administered alone or in combination with one or more therapeutic agents. For example, a subject can be treated with a surfactant alone, or in combination with nucleic acids, chemotherapeutic agents, antibodies, antivirals, steroidal and non-steroidal anti-inflammatories, conventional immunotherapeutic agents, cytokines, chemokines, and/or growth factors. Combinations may be administered either concomitantly (e.g., as an admixture), separately but simultaneously (e.g., via separate intravenous lines into the same subject), or sequentially (e.g., one of the compounds or agents is given first followed by the second). Thus, the term “combination” or “combined” is used to refer to either concomitant, simultaneous, or sequential administration of two or more agents.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Administration of the compositions can be either local or systemic. The pharmaceutical composition can be administered in a number of ways depending on whether local or systemic treatment is desired, and on the area to be treated. Administration may be topically (including ophthalmically, vaginally, rectally, intranasally), orally, by inhalation, intracranially or parenterally (e.g., intravenous drip, subcutaneous, intraperitoneal or intramuscular injection. The disclosed compositions can be administered intravenously, intraperitoneally, intramuscularly, subcutaneously, intracavity, or transdermally).

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Thus, the pharmaceutical carrier for the sonoprotectant and/or other compounds can be a polymeric matrix. U.S. Pat. No. 4,657,543, which is incorporated herein by reference, provides a method for delivering a composition from a polymeric matrix by exposing the polymeric matrix containing the composition to ultrasonic energy. After the polymeric matrix containing the composition or molecule to be released is implanted at the desired location in a liquid environment, such as in vivo, it is subjected to ultrasonic energy to partially degrade the polymer thereby to release the composition or molecule encapsulated by the polymer. The main polymer chain rupture in the case of biodegradable polymers is thought to be induced by shock waves created through the cavitation, which are assumed to cause a rapid compression with subsequent expansion of the surrounding liquid or solid. Apart from the action of shock waves, the collapse of cavitation bubbles is thought to create pronounced perturbation in the surrounding liquid which can possibly induce other chemical effects as well. The agitation may increase the accessibility of liquid molecules, e.g. water, to the polymer. In the case of nondegradable polymers, cavitation may enhance the diffusion process of molecules out of these polymers.

The acoustic energy and the extent of modulation can readily be monitored over wide range of frequencies and intensities. The selection of the parameters will depend upon the particular polymeric matrix utilized in the composition which is encapsulated by the polymeric matrix. The ultrasound frequency or intensity range that is used can be determined empirically, using standard techniques, based on the exposure necessary to result in cavitation and/or the physical effects of ultrasound. Representative suitable ultrasonic frequencies are between about 20 KHz and about 1000 KHz, usually between about 50 KHz and about 200 KHz while the intensities can range between about 1 watt and about 30 watts, generally between about 5 w and about 20 w. The times at which the polymer matrix-composition system are exposed to ultrasonic energy obviously can vary over a wide range depending upon the environment of use. Generally suitable times are between about 1 minute and about 2 hours.

In one aspect, the pharmaceutical carrier for the sonoprotectant and/or other compounds can be a microcapsule. The term “microcapsule” is used herein to mean a small, sometimes microscopic capsule or sphere of organic polymer or other material designed to release its contents when broken by pressure, dissolved, or melted, usually used for slow release drug delivery or to protect orally administered agents from destruction in digestive tract. In one aspect, these microcapsules can be liposomes, microparticles, micelles, microspheres or microbubbles. Previously described microcapsules that can be used with the sonoprotectants disclosed herein are provided as non-limiting examples.

The pharmaceutical carrier for the sonoprotectant and/or other compounds can be a liposome. PCT Application No. WO 92/22298 is incorporated herein by reference for its teaching of methods for the use of liposomes for drug delivery that can be destroyed by irradiation with ultrasound. Provided is a controlled delivery of drugs to a region of a patient wherein the patient is administered a drug containing liposome. Ultrasound is used to determine the presence of the liposomes in the region and to then rupture the liposome to release the drugs in the region. When ultrasound is applied at a frequency corresponding to the peak resonant frequency of the drug containing gas filled liposomes, the liposomes will rupture and release their contents. The peak resonant frequency can be determined by one skilled in the art either in vivo or in vitro by exposing the liposomes to ultrasound, receiving the reflected resonant frequency signals and analyzing the spectrum of signals received to determine the peak, using conventional means. The peak, as so determined, corresponds to the peak resonant frequency (or second harmonic, as it is sometimes termed).

Ultrasound is generally initiated at lower intensity and duration, preferably at peak resonant frequency, and then intensity, time, and/or resonant frequency increased until liposomal rupturing occurs. Although application of the various principles will be readily apparent to one skilled in the art based on the present disclosure, as a general guide for gas filled liposomes of about 1.5 to about 2.0 microns diameter, the resonant frequency will generally be about 750 KHz.

Liposomes described herein may be of varying sizes, but preferably are of a size range wherein they have a mean outside diameter between about 30 nanometers and about 10 microns, with the preferable mean outside diameter being about 2 microns. As is known to those skilled in the art, liposome size influences biodistribution and, therefore, different size liposomes may be selected for various purposes. For intravascular use, for example, liposome size is generally no larger than about 5 microns, and generally no smaller than about 30 nanometers, in mean outside diameter. To provide drug delivery to organs such as the liver and to allow differentiation of tumor from normal tissue, smaller liposomes, between about 30 nanometers and about 100 nanometers in mean outside diameter, are useful. With the smaller liposomes, resonant frequency ultrasound will generally be higher than for the larger liposomes.

The pharmaceutical carrier for the sonoprotectant and/or other compounds can be a microparticle. U.S. Pat. No. 6,068,857, which incorporated herein by reference, provides microparticles containing active ingredients that contain at least one gas or a gaseous phase in addition to the active ingredient(s) and methods for ultrasound-controlled in vivo release of active ingredients. The particles exhibit a density that is less than 0.8 g/cm³, preferably less than 0.6 g/cm³, and have a size in the range of 0.1-8 μm, preferably 0.3-7 μm. In the case of encapsulated cells, the preferred particle size is 5-10 μm. Due to the small size, after i.v. injection they are dispersed throughout the entire vascular system. While being observed visually on the monitor of a diagnostic ultrasound device, a release of the contained substances that is controlled by the user can be brought about by stepping up the acoustic signal, whereby the frequency that is necessary for release lies below the resonance frequency of the microparticles. Suitable frequencies lie in the range of 1-6 MHz, preferably between 1.5 and 5 MHz.

As shell materials for the microparticles that contain gas/active ingredient, basically all biodegradable and physiologically compatible materials, such as, e.g., proteins such as albumin, gelatin, fibrinogen, collagen as well as their derivatives, such as, e.g., succinylated gelatin, crosslinked polypeptides, reaction products of proteins with polyethylene glycol (e.g., albumin conjugated with polyethylene glycol), starch or starch derivatives, chitin, chitosan, pectin, biodegradable synthetic polymers such as polylactic acid, copolymers consisting of lactic acid and glycolic acid, polycyanoacrylates, polyesters, polyamides, polycarbonates, polyphosphazenes, polyamino acids, poly-ξ-caprolactone as well as copolymers consisting of lactic acid and ξ-caprolactone and their mixtures, are suitable. Especially suitable are albumin, polylactic acid, copolymers consisting of lactic acid and glycolic acid, polycyanoacrylates, polyesters, polycarbonates, polyamino acids, poly-ξ-caprolactone as well as copolymers consisting of lactic acid, and ξ-caprolactone.

The enclosed gas(es) can be selected at will, but physiologically harmless gases such as air, nitrogen, oxygen, noble gases, halogenated hydrocarbons, SF₆ or mixtures thereof are preferred. Also suitable are ammonia, carbon dioxide as well as vaporous liquids, such as, e.g., steam or low-boiling liquids (boiling point <37° C.).

The pharmaceutical carrier for the sonoprotectant and/or other compounds can be a polymeric microsphere/microbubble. U.S. Pat. Nos. 5,498,421, 5,635,207, 5,639,473, 5,650,156, and 5,665,382, are incorporated herein by reference for their teaching of the synthesis of polymeric shells containing biologics using high intensity ultrasound. Polymeric microspheres would possess a pharmaceutically viable solution possessing the sonoprotectors in concentrations of between 1 to 100 mM, depending on the sonoprotectors to be used. Microspheres that enter the focal region of the ultrasound beam would rupture due to the physical action of the ultrasonic wave on the microsphere. This would result in the sudden release of sonoprotectors in and in the region of the focal point. The initial relatively high concentration of sonoprotectors encapsulated within the microspheres would be rapidly diluted in the region of treatment to non-toxic levels where the sonoprotectors would still retain their sonoprotecting ability. It would be expected, for example, that the final concentration of sonoprotectors in the region to be treated would instantaneously have to be in the order of 0.1 to 30 mM, depending on the sonoprotecting agent being employed.

A gas space needs to be present so that the bubbles are compressed under the influence of the ultrasonic wave, rupture and release the sonoprotecting agents. Thus, the microbubbles can not be completely filled with solution possessing sonoprotector. Another method, however, is to have a heterogenous mixture of microbubbles that are filled with varying amounts of sonoprotecting solution (from empty bubbles to fully-filled bubbles). In this way, the bubbles possessing less of the sonoprotector solution would violently oscillate and rupture, creating physical forces in the vicinity of partially and fully-filled microbubbles, causing them to rupture.

The pharmaceutical carrier for the sonoprotectant and/or other compounds can be a polymeric micelle. PCT Patent Application No. WO 99/15151 is incorporated herein by reference for its teaching of a method for delivery of a drug to a selected site in a patient using a polymeric micelle. The polymeric micelle can have a hydrophobic core and an effective amount of an encapsulated drug disposed in the hydrophobic core. The application of ultrasonic energy to the selected site can release the drug from the hydrophobic core to the selected site. Polymeric micelles formed by hydrophobic-hydrophilic block copolymers, with the hydrophilic blocks comprised of PEO chains, are very attractive drug carriers. These micelles have a spherical, core-shell structure with the hydrophobic block forming the core of the micelle and the hydrophilic block or blocks forming the shell. Block copolymer micelles have promising properties as drug carriers in terms of their size and architecture.

As a result of the use of microcapsules, i.e., liposomes, microparticles, microbubbles, microspheres, or micelles, combined control of the rate and the site of release of the active ingredients by the user within the entire body can be achieved. This release, by destruction of the microcapsule, can be achieved with ultrasound frequencies that are far below the resonance frequency of the microcapsule with sonic pressures that are commonly encountered in medical diagnosis, without resulting in tissue heating.

An alternative approach, when the frequency of ultrasound required to rupture the microcapsules would be higher than the desired frequency for the ultrasound treatment, is to use other forms of energy to rupture the microcapsules. For example, electricity (Kwon, I. C., et al. Nature 354:291-293, 1991), magnetic fields (Edelman, E. R., et al. J. Biomed. Mater. Res. 19:67-83, 1985), light (Mathiowitz, E. & Cohen, M. D. J. Membr. Sci. 40:67-86, 1989), enzymes (Fischel-Ghodsian, F., et al. Proc. Natl. Acad. Sci. USA 85:2403-2406, 1988), temperature fluctuations (Bae, Y. H., et al. Makromol. Chem. Rapid Commun. 8:481-485, 1987), or pH changes (Siegel, R. A., et al. J. Control. Release 8:179-182, 1988) can be used in place of ultrasound (Kost, J., et al. Proc. Natl. Acad. Sci. USA 86:7663-7666, 1989) to rupture microcapsules comprising the sonoprotectant, all references herein disclosed for their teaching of the rupture of microcapsules with an extrinsic source of energy.

For all compositions and pharmaceutical carriers provided herein, effective dosages and schedules for administration may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. It would be expected that the final concentration of sonoprotectors in the region to be treated would be in the order of 0.1 to 30 mM, depending on the sonoprotecting agent to be employed.

Disclosed herein are methods for protecting cells from ultrasound-mediated cytolysis. The term “ultrasound” is used herein to mean vibrations of the same physical nature as sound but with frequencies above the range of human hearing, i.e., vibrating at frequencies of approximately greater than 20,000 cycles per second (Hz). The term “sonolysis” is used herein to mean a physical/chemical reaction initiated by the formation, growth, oscillations or implosion of cavitation bubbles in liquid, induced by ultrasound. The term “cytolysis” is used herein to mean the pathological breakdown of cells by the destruction of their outer membrane as well as other inducible forms of cell death including, but not limited to, apoptosis and necrosis caused by ultrasound and sonolysis. The use of ultrasound in medicine has diagnostic and therapeutic applications. The term “protecting” as used herein is defined as the reduction of ultrasound-mediated cytolysis to the prevention of ultrasound-mediated cytolysis.

Diagnostic medical ultrasonic imaging is well known, for example, in the common use of sonograms for fetal examination. Ultrasound can also be used to enhance the performance of bioreactors. Therapeutic ultrasound refers to the use of high intensity ultrasonic waves to induce changes in tissue state through both thermal effects (e.g., induced hyperthermia) and mechanical effects (e.g., direct effects of the ultrasonic wave on cells and tissue or indirect effects such as cavitation and acoustic streaming). High frequency ultrasound has been employed in both hyperthermic and cavitational medical applications, whereas low frequency ultrasound has been used principally for its cavitation effect. Examples of therapeutic uses of ultrasound include High Intensity Focused Ultrasound (HIFU), Focused Ultrasound Surgery (FUS), phacoemulsification, sonophoresis (or phonophoresis), thrombolysis, and sonoporation.

Various aspects of diagnostic and therapeutic ultrasound methodologies and apparatus are discussed in depth in an article by G. ter Haar, Ultrasound Focal Beam Surgery, Ultrasound in Med. & Biol., Vol. 21, No. 9, pp. 1089-1100, 1995, and the IEEE Transactions on Ultrasonics, Ferroelectrics, and Frequency Control, November 1996, Vol. 43, No. 6 (ISSN 0885-3010), both of which are incorporated herein by reference for their teaching of medical applications for ultrasound. The IEEE journal is quick to point out that: “The basic principles of thermal effects are well understood, but work is still needed to establish thresholds for damage, dose effects, and transducer characteristics . . . ” Id., Introduction, at page 990.

In the disclosed ultrasound and sonoprotection methods, the cells can be any cells that are cultured in vitro. In one aspect, the disclosed cells can be prokaryotic. In one aspect, the disclosed cells can be eukaryotic. In another aspect, the cells can be any cells within a subject. In one aspect, the subject can be human. In another aspect, the cells can be any healthy cells in the vicinity of a tumor or a thrombus. In another aspect, the cells can be any cells being treated for gene transfection by sonoporation. In another aspect, the cells are non-proliferating cells such as neurons and muscle cells that must be protected from ultrasound mediated cytolysis. In another aspect, the cells can be various forms of plant, animal or microbial cells used in bioreactors.

Described herein are improved methods utilizing ultrasound comprising delivering to the cells, or cells of a subject, any of the surfactants described herein alone or in combination with a pharmaceutically acceptable carrier in conjunction with the administration of ultrasound. As used herein, “delivering to” refers to the administration of the provided composition to, into, or in the vicinity of the target. Delivering to a cell can therefore include, for example, contacting, transfecting, or surrounding a cell. Thus, the provided surfactant can be delivered to regions within a subject that will be treated with ultrasound so as to protect healthy cells that lie in, or in the vicinity of, the region to be treated. As used herein, “in conjunction with” refers to the combination of two or more compositions or methods either concurrently or consecutively. Consequently, in one aspect the provided method comprises delivering to the cells, or cells of a subject, the surfactant(s) prior to the administration of ultrasound. In another aspect, the provided methods comprise delivering to the cells, or cells of a subject, the surfactant(s) concurrent with the administration of ultrasound. The delivery step can further be performed in vitro, in vivo, or ex vivo.

The provided sonoprotection methods are not limited to any particular method or type of ultrasound. For example, the sonoprotection methods and compositions disclosed protect cells in a subject undergoing diagnostic ultrasound. Diagnostic ultrasound can cause capillary lung and intestinal bleeding, which is dependent on the frequency, intensity and duration of ultrasound exposure [Rott, H. D. et al. Ultraschall Med. 18, 226-228 (1997)]. There is a risk of producing unwanted bioeffects, especially in the presence of contrast agents [Barnett, S. B., et al. Ultrasound Med. Biol. 23, 805-812 (1997)], unless strict guidelines for the application parameters of ultrasound for diagnostic purposes are not adhered to [Barnett, S. B. et al. et al. Ultrasound Med. Biol. 26, 355-366 (2000)].

The sonoprotection methods and compositions disclosed protect cells that are in bioreactors. Ultrasound has been shown to enhance the performance of bioreactors through a number of mechanisms. Although sonication is generally associated with the disruption of cells, carefully controlling the ultrasound parameters yields beneficial effects, while minimizing the detrimental effects of ultrasound [Sinisterra, J. V. Ultrasonics 30, 180-185 (1992)]. There is a very narrow window of ultrasound parameters that can be used for obtaining beneficial effects for pollutant destruction by a biological process [Schlafer, O., et al. Ultrasonics 40, 25-29 (2002)]. In a European study on the ultrasound-assisted biological treatment of wastewater for application to the food industry, ultrasound was shown to improve biological activity in laboratory scale reactors [Schlafer, O., et al. Ultrasonics 38, 711-716 (2000)]. However, application of ultrasound above a certain threshold intensity resulted in cavitation and decreased the biological activity well below that observed in the absence of ultrasound [Schlafer, O., et al. Ultrasonics 40, 25-29 (2002)].

In another aspect, the disclosed sonoprotection methods and compositions protect cells adjacent to tumor cells undergoing high intensity focused ultrasound (HIFU). Examples of methods for the use of HIFU have been described in U.S. Pat. No. 6,315,741, which is incorporated herein by reference for its teaching of methods for the in vivo use of HIFU. Disclosed herein are improvements to these methods by the use of any of the surfactants disclosed herein to protect cells of a subject from collateral damage during the use of HIFU to ablate tumors.

Another use of ultrasound to ablate tissue is during phacoemulsification. The technique of phacoemulsification utilizes a small incision, wherein the tip of the instrument is introduced into the eye through this small incision. Localized high frequency waves are generated through this tip to break the cataract into very minute fragments and pieces, which are then sucked out through the same tip in a controlled manner. The ultrasound energy has two main components, a mechanical component which can destroy the cataract, but also a cavitation component which can cause severe disadvantages (Pacifico, R. L. 1994. J. Cataract. Refract. Surg. 20, 338-341). Cavitation bubbles formed during phacoemulsifaction result in the formation of free radicals (Topaz, M. et al. 2002. Ultrasound Med. Biol. 28, 775-784), which are believed to be a source of damage to the corneal endothelium (Holst, A., Rolfsen, W., Svensson, B., Ollinger, K. & Lundgren, B. 1993. Curr. Eye Res. 12, 359-365; Takahashi, H. et al. 2002. Arch. Opthalmol. 120, 1348-1352). Viscoelastic substances are used in cataract surgery to help prevent corneal endothelial cell loss (Hessemer, V. & Dick, B. 1996. Klinische Monatsblat. Augenheilkunde 209, 55-61). Sonoprotective agents can therefore be used either in combination with current viscoelastic substances or as an ingredient in a whole new branch of protective liquid mixtures during phacoemulsification. One benefit of this is a reduction in the viscosity of the additive necessary for protection of the corneal endothelial cells, thereby allowing for easier aspiration but at the same time, superior protection from the detrimental effects of ultrasound. Superior protection properties can also allow for higher ultrasound intensities to be used, thereby reducing treatment time.

In high-intensity focused ultrasound (HIFU) hyperthermia treatments, the intensity of ultrasonic waves generated by a highly focused transducer increases from the source to the region of focus where very high temperatures can be reached, e.g. 98° C. The absorption of the ultrasonic energy at the focus induces a sudden temperature rise of tissue—as high as one to two hundred degrees Kelvin/second—which causes the irreversible ablation of the target volume of cells in the focal region. Thus, for example, HIFU hyperthermia treatments can cause necrotization of an internal lesion without damage to the intermediate tissues. The focal region dimensions are referred to as the depth of field, and the distance from the transducer to the center point of the focal region is referred to as the depth of focus. In the main, ultrasound is a promising non-invasive surgical technique because the ultrasonic waves provide a non-effective penetration of intervening tissues, yet with sufficiently low attenuation to deliver energy to a small focal target volume. Currently there is no other known modality that offers noninvasive, deep, localized focusing of non-ionizing radiation for therapeutic purposes. Thus, ultrasonic treatment has a great advantage over microwave and radioactive therapeutic treatment techniques.

In addition to the use of HIFU to ablate tissue, also considered is the beneficial use of HIFU under more controlled conditions of ultrasound application in the reversible and non-destructive disruption the blood brain barrier (BBB) [Mesiwala, A. H. et al. Ultrasound Med. Biol. 28, 389-400 (2002)], incorporated herein by reference for the use of HIFU to disrupt the BBB. A large problem with this technique is that irreparable damage to the tissue of the brain can occur. Sonoprotectors can protect against such damage while allowing the reversible disruption of the BBB.

A major issue facing the use of HIFU techniques is cavitation effects. Cavitation can occur in at least three ways important for consideration in the use of ultrasound for medical procedures. The first is gaseous cavitation, where dissolved gas diffuses into cavitation bubbles during a negative pressure phase of an acoustic wave. The second is vaporous cavitation due to the negative pressure amplitude of the wave becoming low enough for a fluid to convert to its vapor form at the ambient temperature of the tissue fluid. The third is where the ultrasonic energy is absorbed to an extent to raise the temperature above boiling at ambient pressure. At lower frequencies, the time that the wave is naturally in the negative pressure phase is longer than at higher frequencies, providing greater time for gas or vapor containing cavitation bubbles to be formed. All other factors being equal, exposure at lower frequency requires lower pressure amplitudes in order for cavitation bubbles to be formed, compared to higher frequencies of ultrasound. Higher frequencies are more rapidly absorbed and therefore raise the temperature more rapidly for the same applied intensity than a lower frequency. Thus, gaseous and vaporous cavitation are promoted by low frequencies and boiling cavitation by high frequency. However, both types of cavitation can occur at all frequencies depending on the mode of irradiation, for example, time of ultrasound exposure, pulsed or continuous exposure regimes, etc.

For HIFU applications it has been found that ultrasonically induced cavitation occurs when an intensity threshold is exceeded such that tensile stresses produced by acoustic rarefaction generates vapor cavities within the tissue itself. Subsequent acoustic cycles cause bubbles to oscillate around a mean position and may cause bubbles to grow to a size where they can undergo inertially driven collapse; because non-condensing gases are created, there are strong radiating pressure forces that exert high shear stresses. Consequently, the tissue can shred or be pureed into an essentially liquid state. Control of such effects has yet to be realized for practical purposes; hence, it is generally desirable to avoid tissue damaging cavitation whenever it is not a part of the intended treatment.

For HIFU, the focused ultrasound may be produced in any manner. The ultrasound transducers are preferably operated while varying one or more characteristics of the ablating technique such as the frequency, power, ablating time, and/or location of the focal axis relative to the tissue. For example, the transducer can be operated at a frequency of 2-7 MHz and a power of 80-140 watts for 0.01-1.0 second. The transducer can be operated at a frequency of 2-14 MHz at a power of 20-60 watts for 0.7-4 seconds. The ultrasonic transducer can also be activated at a at a frequency of 6-16 MHz at 2-10 watts until the near surface NS temperature reaches 70-85° C.

Another field of HIFU use is as a direct surgical tool for non-invasive surgical procedures, i.e., Focused Ultrasound Surgery (FUS). Ultrasound can be used as an electromechanical driver for cutting tool implementations, e.g., U.S. Pat. No. 5,324,299 to Davison et al., incorporated herein by reference for its teaching of an ultrasonic scalpel blade, sometimes referred to as a “harmonic scalpel,” and its uses).

Any of the surfactants described herein can be used alone or in combination with other surfactants to protect cells from ultrasound-mediated cytolysis that occurs during, for example, HIFU. In one aspect, the surfactant used to protect cells from ultrasound-mediated cytolysis comprises a carbohydrate having at least one hydrophobic group. In another aspect, the surfactant has at least one unit having the formula I described above. In a further aspect, the surfactant is hexyl-β-D-glucopyranoside, heptyl-β-D-glucopyranoside, octyl-β-D-glucopyranoside, nonyl-β-D-glucopyranoside, hexyl-β-D-maltopyranoside, n-octyl-β-D-maltopyranoside, n-octyl-β-D-thioglucopyranoside, 2-propyl-1-pentyl-β-D-maltopyranoside, methyl-6-O—(N-heptylcarbamoyl)-α-D-glucopyranoside, 3-cyclohexyl-1-propyl-β-D-glucoside, or 6-O-methyl-n-heptylcarboxyl-α-D-glucopyranoside.

In another aspect, described herein are methods for delivering a compound to a cell. In one aspect, the method involves:

(a) delivering to the cells a composition comprising any surfactant described herein, wherein the surfactant accumulates at the gas/liquid interface of cavitation bubbles, wherein the surfactant quenches a radical; and
(b) subjecting the cells to ultrasound frequencies sufficient to sonoporate the cells in the presence of the compound, thereby delivering the compound to the cells.

The surfactants described herein can facilitate the delivery of a compound into a cell. The provided methods are not limited to a particular cell type or location. The term “compound” is defined herein to include any bioactive material such as, for example, a nucleic acid, a protein, or small molecule (e.g., pharmaceutical). Thus, sonoporation can be used for gene therapy to transfect the cell with naked or plasmid DNA [Fechheimer, M. et al. Proc. Natl. Acad. Sci. U.S.A. 84, 8463-8467 (1987)]. Sonoporation can also be used to transport a relatively large drug molecule across the plasma membrane [Miller, M. W. Ultrasound Med. Biol. 26, S59-S62 (2000)]. Thus, in one aspect of the method, the disclosed compound is a nucleic acid being delivered to cells of a subject. In another aspect, the delivery of the nucleic acid is for the purpose of gene therapy. Thus, provided are improved methods of gene therapy wherein the gene can be delivered by sonoporation and wherein a sonoprotectant is administered in conjunction with the gene. In another aspect, the nucleic acid is being delivered to non-proliferating cells within a subject, such as neurons or muscle cells, which cannot afford to be damaged during sonoporation.

Methods involving nucleic acid based delivery systems are well known in the art. Briefly, transfer vectors can be any nucleotide construct used to deliver genes into cells (e.g., a plasmid), or as part of a general strategy to deliver genes, e.g., as part of recombinant retrovirus or adenovirus (Ram et al. Cancer Res. 53:83-88, (1993)). As used herein, plasmid or viral vectors are agents that transport the disclosed nucleic acids into the cell without degradation and include a promoter yielding expression of the gene in the cells into which it is delivered. In some embodiments the promoters are derived from either a virus or a retrovirus. The nucleic acids that are delivered to cells typically contain expression controlling systems. For example, the inserted genes in viral and retroviral systems usually contain promoters, and/or enhancers to help control the expression of the desired gene product.

A promoter is generally a sequence or sequences of DNA that function when in a relatively fixed location in regard to the transcription start site. A promoter contains core elements required for basic interaction of RNA polymerase and transcription factors, and may contain upstream elements and response elements. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. The transcribed units can contain other standard sequences alone or in combination with the above sequences improve expression from, or stability of, the construct.

The delivery step can be performed in vitro, in vivo, or ex vivo using techniques known in the art. After step (a), ultrasound radiation is applied with an intensity and for a period of time effective to sonoporate the cells. The term “sonoporate” as used herein refers to the application of ultrasound to a living surface that is acting as a barrier (e.g., skin of a subject or the plasma membrane of a cell) for temporarily permeabilising the barrier so as to facilitate the entry of large or hydrophilic molecules (e.g., a drug or nucleic acid). The use of “sonoporation” is not meant to be limited to a specific mechanism by which the barrier is permeabilized except as to indicate that ultrasound is the initiator. For example, as used herein, sonoporation comprises the permeabilization of a living barrier, such as the lipid membrane, due, at least in part, to the collapse of contrast agents, ultrasound-induced microbubbles and/or the physical effects of ultrasound and acoustic cavitation.

The effects of both sonoporation and sonoprotection are dependent upon the specific barrier, i.e., cell type and environment that is targeted. However, the optimum frequency can be routinely and empirically determined for each cell type and sonoprotectant being used. In one aspect, the frequency of ultrasound used for sonoportation is between 20 kHz and 5 MHz.

Sonoporation is recognized as a method for the transfection of genes into cultured cells (Miller D L, et al. Somat Cell Mol. Genet. 2002 November; 27(1-6):115-34), incorporated herein by reference for its teaching of methods for the delivery of nucleic acid to cells by sonoporation. Ultrasound has been used with contrast agents such as for example, Optison or Albunex, which enhance the sonoporation effect, to transfect a variety of cell lines with naked plasmid DNA in vivo as well as in vitro (Taniyama Y, et al. Gene Ther. 2002 March; 9(6):372-80), incorporated herein by reference for their teaching of sonoporation. Sonoporation results in the formation of transient holes (typically less than 5 μm) in the cell surface, which explains the rapid migration of transgenes into the cells. Difficulties with concomitant cell death in many of these studies have highlighted the need for methods of protecting the cells from the deleterious chemical effects of ultrasound, e.g., radical damage, while still allowing the mechanical formation of pores in the cell membrane for gene transfection.

As used herein, “sonophoresis” refers to a subtype of sonoporation whereby ultrasound is used to increase the penetration of compounds through the skin and other biological membranes. U.S. Pat. No. 5,421,816, U.S. Pat. No. 5,618,275, U.S. Pat. No. 6,712,805 and U.S. Pat. No. 6,487,447, are incorporated herein by reference for their teaching of ultrasound mediated delivery of compounds through the skin.

Transdermal and/or intradermal delivery of compounds such as drugs offer several advantages over conventional delivery methods including oral and injection methods. It is a non-invasive, convenient, and painless method for the delivery of a predetermined drug dose to a localized area with a controlled steady rate and uniform distribution.

Transdermal and/or intradermal delivery of compounds require transport of the compounds through the stratum corneum, i.e., the outermost layer of the skin. The stratum corneum provides a formidable chemical barrier to any chemical entering the body and only small molecules having a molecular weight of less than 500 Da (Daltons) can passively diffuse through the skin at rates resulting in therapeutic effects. A Dalton is defined as a unit of mass equal to 1/12 the mass of a carbon-12 atom, according to “Steadman's Electronic Medical Dictionary” published by Williams and Wilkins (1996). Thus, ultrasound is used to provide openings in the skin through which larger molecules can be delivered.

Sonophoresis is limited by the range of ultrasound parameters that can be applied for its safe use [Mitragotri, S. & Kost, J. Adv. Drug Deliv. Rev. 56, 589-601 (2004)]. “Low frequency ultrasound” for sonophoresis has been described, and is provided herein, as lying in the range from approximately 20 kHz to 450 kHz [Mitragotri, S. & Kost, J. Adv. Drug Deliv. Rev. 56, 589-601 (2004); Mutoh, M. et al. J. Control. Release 92, 137-146 (2003)]. For low frequency ultrasound, acoustic cavitation is the main mechanism by which sonophoresis operates [Merino, G., et al. J. Pharm. Sci. 92, 1125-1137 (2003); Lavon, A. & Kost, J. Drug Discov. Today 9, 670-676 (2004); Mitragotri, S. & Kost, J. Adv. Drug Deliv. Rev. 56, 589-601 (2004)]. Since the stratum corneum (SC) has a thickness of approximately 15 μm, cavitation cannot occur within the SC at these frequencies, since the resonance radius of bubbles at 20 to 100 kHz is 10 to 100 μm [Mitragotri, S. & Kost, J. Adv. Drug Deliv. Rev. 56, 589-601 (2004)]. Instead, cavitation can occur in the coupling medium between the skin and the transducer. Spherical collapse of the bubbles near the surface of the SC produces shock waves that can disrupt the SC lipid bilayer, whereas high speed liquid jetting from the asymmetric collapse of cavitation-bubbles on the SC can penetrate into the SC, thereby disordering lipids of the SC and opening aqueous transport channels [Lavon, A. & Kost, J. Drug Discov. Today 9, 670-676 (2004); Mitragotri, S. & Kost, J. Adv. Drug Deliv. Rev. 56, 589-601 (2004)].

Evidence also exists of the possibility of cavitation occurring in the SC when high frequency ultrasound is used, since the resonance size of the bubbles are relatively small (less than 3 microns) [Machet, L. & Boucaud, A. et al. Int. J. Pharm. 243, 1-15 (2002)]. However, the safety aspects of both high and low frequency sonophoresis have not yet been addressed [Lavon, A. & Kost, J. Drug Discov. Today 9, 670-676 (2004)]. Low frequency cavitation is known to be associated with the formation of radicals and bubbles collapsing near or on the SC will not only produce mechanical effects, but potentially damaging free radical effects to the SC.

Thus, provided is an improved method of performing in vivo sonophoresis of a skin area and transdermal and/or intradermal delivery of a compound. Sonophoresis allows the painless and rapid delivery of compounds such as, for example, drugs through the skin for either topical or systemic therapy. In one aspect, the method includes administering to the skin any of the surfactants provided herein in a pharmaceutically accepted carrier.

In one example, the method further includes providing a container containing a predetermined amount of the drug solution and having a first end and a second end, the second end being covered with a porous membrane can be used. Next, a tip of an ultrasound horn is submerged in the drug solution through the first end of the container and then the porous membrane is placed in contact with the skin area. The ultrasound radiation is applied with an intensity, for a period of time, and at a distance from the skin area effective to generate cavitation bubbles. In one aspect, the frequency of ultrasound is between 20 kHz and 5 MHz. In another aspect, the ultrasound frequency is between 20 kHz and 500 kHz. The cavitation bubbles collapse and transfer their energy into the skin area thus causing the formation of pores in the skin area. The ultrasound radiation intensity and distance from the skin area are also effective in generating ultrasonic jets, which ultrasonic jets then drive the drug solution through the porous membrane and the formed pores into the skin area.

Any of the surfactants described herein can be used alone or in combination with other surfactants to protect cells from ultrasound-mediated cytolysis that occurs during sonoporation. In one aspect, the surfactant comprises a carbohydrate having at least one hydrophobic group. In another aspect, the surfactant has at least one unit having the formula I described above. In a further aspect, the surfactant is hexyl-β-D-glucopyranoside, heptyl-β-D-glucopyranoside, octyl-β-D-glucopyranoside, nonyl-β-D-glucopyranoside, hexyl-β-D-maltopyranoside, n-octyl-β-D-maltopyranoside, n-octyl-β-D-thioglucopyranoside, 2-propyl-1-pentyl-β-D-maltopyranoside, n-octyl-α-D-glucopyranoside, methyl-6-O—(N-heptylcarbamoyl)-α-D-glucopyranoside, 3-cyclohexyl-1-propyl-β-D-glucoside, or 6-O-methyl-n-heptylcarboxyl-α-D-glucopyranoside.

In another aspect, disclosed herein are methods of enhancing the metabolic activity of cells in a bioreactor. In one aspect the method involves:

(a) delivering to the cells a composition comprising any surfactant described herein, wherein the surfactant accumulates at the gas/liquid interface of cavitation bubbles, wherein the surfactant quenches a radical; and

(b) subjecting the cells to ultrasound frequencies sufficient to enhancing the metabolic activity of cells in a bioreactor.

Bioreactors comprise plant, animal or microbial cells whose metabolic activity dictates the efficiency of the particular process. Enhancing the metabolic activity of these cells can greatly enhance the efficacy of biotechnological processes. Ultrasound has been shown to enhance the performance of bioreactors through a number of mechanisms. Although sonication is generally associated with the disruption of cells, carefully controlling the ultrasound parameters yields beneficial effects, while minimizing the detrimental effects of ultrasound (Sinisterra, J. V. 1992. Ultrasonics 30, 180-185). There is, however, a very narrow window of ultrasound frequencies that can be used for obtaining beneficial effects for pollutant destruction by a biological process (Schlafer, O., et al. 2002. Ultrasonics 40, 25-29).

The addition of sonoprotectors can allow a more flexible use of ultrasound intensities, making the choice of ultrasound power for the process less critical. This can result in the beneficial effects of cavitation induced physical processes (such as acoustic streaming for enhanced mixing and mass transport) while protecting microbes from ultrasound induced inactivation. The optimum frequency can thus be routinely and empirically determined for each cell type and sonoprotectant being used. In general, the frequency of ultrasound is between 20 kHz and 5 MHz. Furthermore, since different cells are known to have different susceptibilities to ultrasound damage (Chisti, Y. 2003. Trends Biotechnol. 21, 89-93), sonoprotectors can protect a diverse population of microbes from ultrasound inactivation, thereby allowing organisms with different pollutant degradation pathways to operate simultaneously in the one system.

In another aspect, disclosed herein is a method of treating a tumor in a subject in need of such treatment, comprising (a) administering to the area of the tumor an effective amount of a surfactant, wherein the surfactant accumulates at the gas/liquid interface of cavitation bubbles, wherein the surfactant quenches a radical; and subjecting the tumor to high intensity focused ultrasound (HIFU), whereby the tumor is treated. By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.). “Treatment” or “treating” means to administer a composition to a subject or a system with an undesired condition. The effect of the administration of the composition to the subject can have the effect of but is not limited to reducing or preventing the symptoms of the condition, a reduction in the severity of the condition, or the complete ablation of the condition. By “effective amount” is meant a therapeutic amount needed to achieve the desired result or results. The effects of both HIFU and sonoprotection are dependent upon the specific cell type and environment that is targeted. However, the optimum frequency can be routinely and empirically determined for each cell type and sonoprotectant being used. In general, the frequency of ultrasound is between 20 kHz and 5 MHz.

Any of the various types of ultrasound devices, including diagnostic ultrasound imaging devices, may be employed in the practice of the invention, the particular type or model of the device not being critical to the method of the invention. Also suitable are devices designed for administering ultrasonic hyperthermia, such devices being described in U.S. Pat. Nos. 4,620,546, 4,658,828, and 4,586,512, the disclosures of each of which are hereby incorporated herein by reference in their entirety. Preferably, the device employs a resonant frequency (RF) spectral analyzer. Also suitable are ultrasound devices designed to contact the target cells or tissues directly via a probe. These devices can be used to target ultrasound to internal organs or tissues during, for example, HIFU or sonoporation. The sonoprotectants of the invention can be directed to these organs and tissues via the same portals using the disclosed means.

Tumors that can be treated by HIFU and sonoprotection can include for example uterine leiomyoma, breast tumor, prostate cancer, benign prostatic hyperplasia, liver tumor, kidney tumor; brain tumor; primary malignant bone tumor, tumors of the lymphnode, lung and pleura, pancreas, soft tissue and adrenal tumors.

Modes of administration of the sonoprotectant can include for example transvaginal treatment, transrectal treatment, transcranial treatment, inhalation to the lung, or injection into the heart.

Any of the surfactants described herein can be used alone or in combination to protect cells from ultrasound-mediated cytolysis that occurs during the treatment of tumors. In one aspect, the surfactant used to treat a tumor in a subject comprises a carbohydrate having at least one hydrophobic group. In another aspect, the surfactant has at least one unit having the formula I described above. In a further aspect, the surfactant is hexyl-β-D-glucopyranoside, heptyl-β-D-glucopyranoside, octyl-β-D-glucopyranoside, nonyl-β-D-glucopyranoside, hexyl-β-D-maltopyranoside, n-octyl-β-D-maltopyranoside, n-octyl-β-D-thioglucopyranoside, 2-propyl-1-pentyl-β-D-maltopyranoside, n-octyl-α-D-glucopyranoside, methyl-6-O—(N-heptylcarbamoyl)-α-D-glucopyranoside, 3-cyclohexyl-1-propyl-β-D-glucoside, or 6-O-methyl-n-heptylcarboxyl-α-D-glucopyranoside.

In another aspect, disclosed herein is a method for protecting cells from ultrasound-mediated cytolysis comprising administering to the cells any of the surfactants described herein, wherein the surfactant accumulates at the gas/liquid interface of cavitation bubbles, wherein the surfactant quenches radicals. The phrase “quenches a radical” is defined herein as the ability of the surfactant to reduce the concentration of radicals present in a cavitation bubble. Reactive radicals include, but are not limited to, primary radicals, cytotoxic radicals, or precursors of cytotoxic radicals. Examples of primary radicals include, but are not limited to, H. and HO..

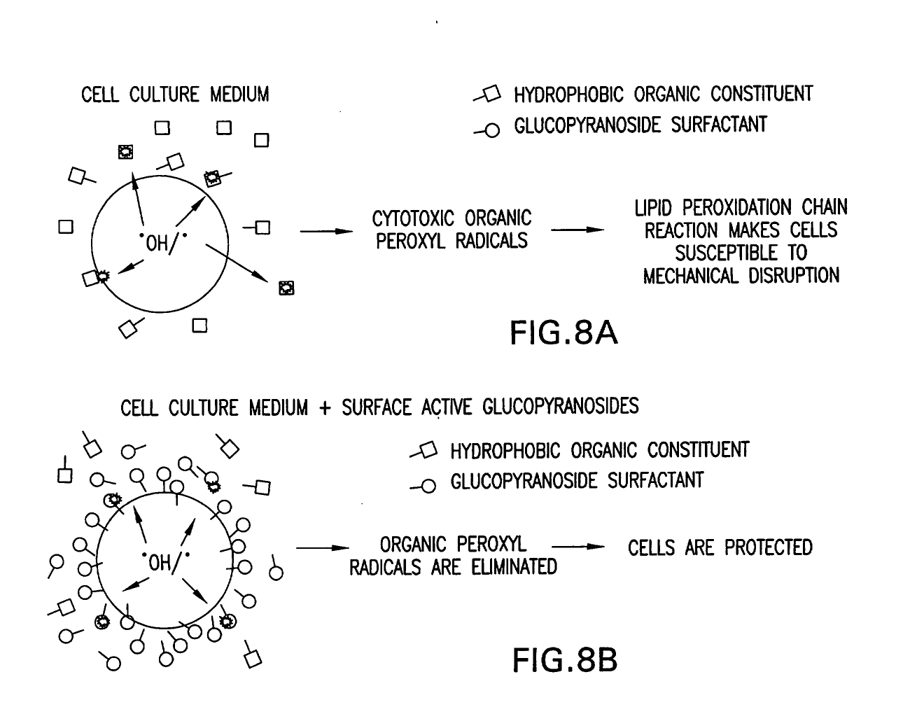

Not wishing to be bound by theory, it is believed that the first step in a quenching mechanism of a surfactant provided herein involves the rapid abstraction of a hydrogen atom of the surfactant by reactive radicals. In the case when the surfactant is an alkylated carbohydrate, hydrogen abstraction from a ring carbon occurs in preference to abstraction of a hydrogen atom from the alkyl chain of the surfactant, which is in competition with reactions of the radicals with the hydrophobic components of the cell culture medium (see FIG. 8b). This significantly reduces the number of carbon-centered radicals formed on the hydrophobic components of the cell culture medium to which oxygen could otherwise rapidly add to produce cytotoxic substrate derived reactive oxygen species, such as organic peroxyl radicals, that could damage the cell membrane. Thus, the surfactant is quenching (i.e., reducing the concentration of) deleterious radicals. For example, D-glucose can undergo relatively rapid hydrogen abstraction reactions with hydroxyl radicals in aqueous solutions [Bothe, Schuchmann and von Sonntag, 1977]. Oxygen rapidly adds to carbon-centered radicals formed on the glucopyranoside ring to form mainly α-hydroxy peroxyl radicals. However, α-hydroxy peroxyl radicals formed on the ring structure of the glucopyranosides are relatively short lived due to either the rapid elimination of the hydroperoxyl radical (HO.₂), or fragmentation reactions due to bimolecular reactions of peroxyl radicals The rate of these reactions can be as fast as diffusion controlled and depend on a number of variables, namely the site of H-abstraction from the glucopyranoside ring and the concentration of oxygen.

Although the elimination reaction described above involves the formation of hydroperoxyl radicals, at neutral pH, hydroperoxyl radicals decompose via a disproportionation reaction with superoxide to produce H₂O₂. In comparison to substrate derived reactive oxygen species, such as peroxyl radicals, relatively low concentrations of H₂O₂ formed in this way would not be expected to be as effective at initiating lipid peroxidation chain reactions in the cell membrane.

The above mechanism offers one possible explanation of how the yield of cytotoxic organic peroxyl radicals and other substrate derived reactive oxygen species are decreased in the presence of the disclosed sonoprotectants during sonolysis, thereby protecting cells from ultrasound induced cytolysis.

The ability of the surfactants to quench harmful radicals produced by ultrasound is based in part on their ability to accumulate at the gas/liquid interface of cavitation bubbles. The hydrophilic end of the surfactant is strongly attracted to the water molecules and the force of attraction between the hydrophobic group and water is only slight. Therefore, while not wishing to be bound by theory, it is believed that surfactant molecules can adsorb at the gas/solution interface of cavitation bubbles after aligning themselves so that the hydrophilic end of the surfactant is generally toward the water and the hydrophobic end points towards the gas/liquid interface of the cavitation bubble. Following the violent collapse of cavitation bubbles, the adsorbed molecules are randomly distributed throughout the interfacial region of the hot spot, which has different properties (for example, high temperature and pressure, low dielectric constant) compared to that of the interfacial region of cavitation bubbles under ambient conditions.

Suitable ultrasonic frequencies that can be used herein are generally between about 20 KHz and about 10 MHz, usually between about 20 KHz and about 1 MHz. Intensities can range between about 0.1 watt and about 150 watts, generally between about 5 w and about 20 w. The duration can vary over a wide range depending upon the environment of use. Generally, suitable times are between about 1 second and about 2 hours. Other suitable ultrasound exposure conditions are known in the art and provided herein. The preferred exposure conditions for target cell(s) and surfactant, or combination thereof, can be empirically determined.

The concentration of the surfactants described herein can, for example, be in the range of 0.1 to about 100 mM, including but not limited to 0.1, 0.2, 0.3, 0.4, 0.5, 0.6, 0.7, 0.8, 0.9, 1, 2, 3, 4, 5, 6, 7, 8, 9, 10, 15, 20, 25, 30, 40, 50, 60, 70, 80, 90, and 100 mM. Any of the herein provided surfactants can be used, either alone or in combination. Suitable concentrations for protecting target cells can be empirically determined.

Any of the surfactants described herein can be used alone or in combination with other solutes that promote the adsorption of sonoprotectors to the gas/solution interface of cavitation bubbles. For example, certain impurities (for example octanol) are known to promote adsorption of surfactants to the gas/solution interface and salts are known to promote adsorption of ionic surfactants to the gas/solution interface.

Any of the surfactants described herein can be used alone or in combination with one or more other surfactants to protect cells from ultrasound-mediated cytolysis by quenching a radical. In one aspect, the surfactants comprise a carbohydrate having at least one hydrophobic group. In another aspect, the surfactants have at least one unit having the formula I described above. In a further aspect, the surfactants are a combination of hexyl-β-D-glucopyranoside, heptyl-β-D-glucopyranoside, octyl-β-D-glucopyranoside, nonyl-β-D-glucopyranoside, hexyl-β-D-maltopyranoside, n-octyl-β-D-maltopyranoside, n-octyl-β-D-thioglucopyranoside, 2-propyl-1-pentyl-β-D-maltopyranoside, n-octyl-α-D-glucopyranoside, methyl-6-O—(N-heptylcarbamoyl)-α-D-glucopyranoside, 3-cyclohexyl-1-propyl-β-D-glucoside, or 6-O-methyl-n-heptylcarboxyl-α-D-glucopyranoside. Thus, the surfactants can be a combination of, for example, hexyl-β-D-glucopyranoside and heptyl-β-D-glucopyranoside, octyl-β-D-glucopyranoside and nonyl-β-D-glucopyranoside, hexyl-βD-maltopyranoside and n-octyl-β-D-maltopyranoside, n-octyl-β-D-thioglucopyranosid and 2-propyl-1-pentyl-β-D-maltopyranoside, n-octyl-α-D-glucopyranoside and methyl-6-O—(N-heptylcarbamoyl)-α-D-glucopyranoside, 3-cyclohexyl-1-propyl-β-D-glucoside and 6-O-methyl-n-heptylcarboxyl-α-D-glucopyranoside, heptyl-β-D-glucopyranoside and octyl-β-D-glucopyranoside, nonyl-β-D-glucopyranoside and hexyl-β-D-maltopyranoside, n-octyl-β-D-maltopyranoside and n-octyl-β-D-thioglucopyranoside, 2-propyl-1-pentyl-β-D-maltopyranoside and n-octyl-α-D-glucopyranoside, methyl-6-O—(N-heptylcarbamoyl)-α-D-glucopyranoside and 3-cyclohexyl-1-propyl-β-D-glucoside, 6-O-methyl-n-heptylcarboxyl-α-D-glucopyranoside and hexyl-β-D-glucopyranoside, hexyl-β-D-glucopyranoside and octyl-β-D-glucopyranoside, nonyl-β-D-glucopyranoside and n-octyl-β-D-maltopyranoside, n-octyl-β-D-thioglucopyranoside and n-octyl-α-D-glucopyranoside, methyl-6-O—(N-heptylcarbamoyl)-α-D-glucopyranoside and 6-O-methyl-n-heptylcarboxyl-α-D-glucopyranoside, heptyl-β-D-glucopyranoside and nonyl-β-D-glucopyranoside, hexyl-β-D-maltopyranoside and n-octyl-β-D-thioglucopyranoside, 2-propyl-1-pentyl-β-D-maltopyranoside and methyl-6-O—(N-heptylcarbamoyl)-α-D-glucopyranoside, or 3-cyclohexyl-1-propyl-β-D-glucoside and hexyl-β-D-glucopyranoside.

Combinations of surfactants can be at any ratio. As an example, for a given combination, a surfactant can be from about 0.001% to 99.999% of the total concentration of surfactant. Suitable concentrations for protecting target cells can be empirically determined.

As disclosed herein, the optimal glucopyranoside and concentration thereof and the preferred frequency of ultrasound that would result in sonolysis of one cell type but be sonoprotective for another cell type is a matter of selection. Thus, provided is a method of selecting a surfactant for sonoprotection of a cell or cells in a mixed (heterogeneous) population of cells, comprising starting with a mixed cell culture comprising at least a first and second cell type, adding to the culture the surfactant, or combination of surfactants, at a given concentration(s), exposing the cells to ultrasound at a given frequency, intensity and duration, and monitoring the survival of the first and second cell types.

Further provided is a method of selectively killing a first cell type located in a mixed population of cell types, while simultaneously protecting a second cell type, comprising administering to the cells a suitable surfactant, or combination of surfactants, at a suitable concentration(s) identified by the herein provided selection method, and exposing the cells to suitable ultrasound conditions identified herein for the first and second cell types, wherein the ultrasound conditions sonolyse the first cell type, and wherein the surfactant protects the second cell type from sonolysis.

For example, provided is a method of selectively killing target cells, such as leukemia cells, while protecting the remaining cells within a patients blood, comprising isolating a patients blood, administering to the blood a suitable surfactant, or combination of surfactants, at a suitable concentration(s) identified by the herein provided selection method, and exposing the blood to suitable ultrasound conditions identified herein for the cells, wherein the ultrasound conditions sonolyse the target cells, and wherein the surfactant protects the remaining cells from sonolysis, filtering the surfactants out of the blood, and administering the blood back to the patient.

Sonoprotecting surfactants can also be selected that can protect healthy tissue from the cavitation effects of ultrasound, but which do not effectively protect diseased tissue from cavitation induced sonolysis. For example, HIFU treatment can be combined with a selective sonoprotectant, such that diseased tissue is killed by both ablation and sonolysis, while the surrounding healthy tissue is protected from sonolysis. Suitable concentrations for protecting target cells can be empirically determined. Likewise, preferred exposure conditions for target cell(s) and surfactant, or combination thereof, can be empirically determined

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### N-alkyl-glucopyranosides Protect HL-60 Cells from Ultrasound-Induced Cytolysis

Chemicals: the nitroso spin trap 3,5-dibromo-4-nitrosobenzenesulfonic acid-sodium salt (DBNBS) was obtained from Sigma-Aldrich. Dulbeco's phosphate buffered saline (DPBS, pH=7.4) was obtained from Biofluids. Methyl-β-D-Glucopyranoside (MGP) was obtained from Sigma-Aldrich; hexyl-β-D-Glucopyranoside (HGP, ≧98%), heptyl-β-D-Glucopyranoside (HepGP, >98%), octyl-β-D-Glucopyranoside (OGP, ≧99%) and decyl-β-D-Glucopyranoside (DGP, ≧99%) were obtained from Fluka; n-octyl-α-D-glucopyranoside (alphaOGP), methyl-6-O—(N-heptylcarbamoyl)-α-D-glucopyranoside (ANAMEG-7), 3-cyclohexyl-1-propyl-β-D-glucoside(Cyglu-3), 6-O-methyl-n-heptylcarboxyl-α-D-glucopyranoside (MHC-alpha-GP) were obtained from Anatrace, Inc., Maumee, Ohio, USA.

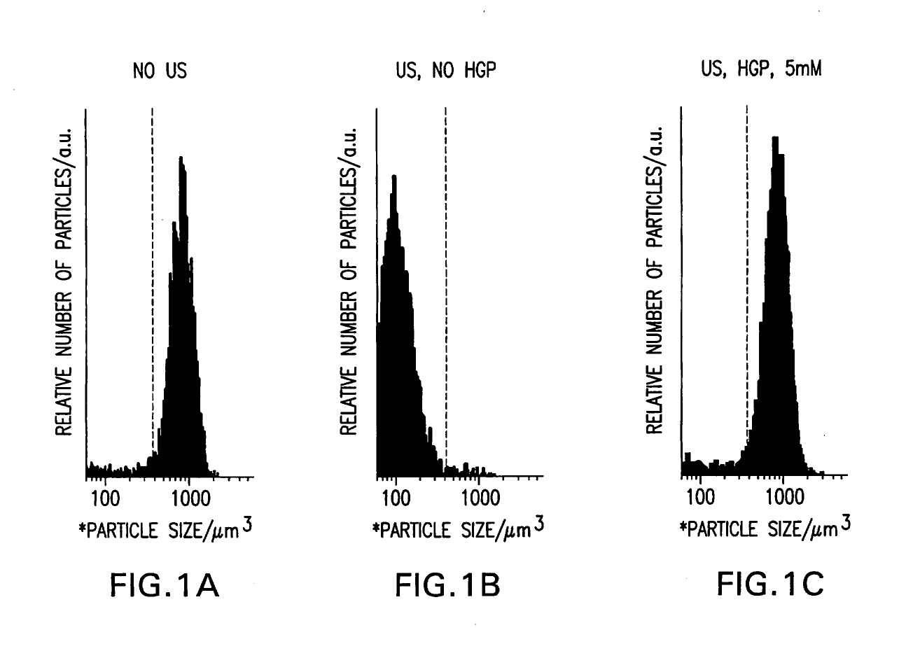

Cells: HL-60 myeloid leukemia cells (American Type Culture Collection) were grown in a suspension of RPMI 1640 medium (GIBCO, Gaithersburg, Md.) containing 10% calf serum. The population of HL-60 cells doubled every 23±1 hr (hour ±SEM) when incubated at 37° C. in a CO₂ (5%) containing atmosphere. Cells were harvested, re-suspended in fresh RPMI medium and kept at 25° C. until the start of the experiment (typically less than 1 hr). The cell concentration was kept constant in all experiments (≈5×10⁵ cells/ml) because of the possible effect of cell concentration on ultrasonically induced cell lysis (Brayman, A. A. et al. 1996). The fraction of intact cells before and after ultrasound was determined using a Coulter multisizer (model IIe) connected to a sampling stand (model IIa). The number of intact cells was determined by counting the total number of particles under the bell shaped curve (e.g., FIG. 1a) before and following sonolysis. The cytolysis percentage was determined by subtracting the number of intact cells following sonolysis from the number of intact cells before sonolysis. This value was divided by the number of intact cells before sonolysis and multiplied by 100 to obtain the cytolysis percentage value.

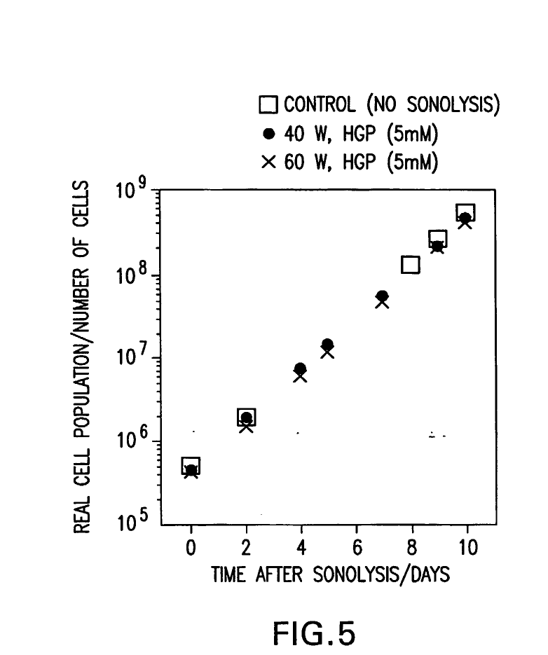

Reproduction Assay: for FIG. 5, a reproduction assay was conducted over a period of 10 days to determine the long term viability of cells following ultrasound treatment in the presence of either HGP or OGP at concentrations where 100% protection from cytolysis occurred. The long term viability of treated cell suspensions was compared to the long term viability of untreated control cell suspensions held under exactly the same conditions (FIG. 5). Immediately following sonolysis, a 100 μl aliquot of the 1 ml treated (or control) samples was used for Coulter counter analysis in order to confirm that 100% of the cells had survived the ultrasound treatment. Viability of the cells was determined by using a very small volume (approx. 20 μl) of suspension for trypan blue staining. The remaining ≈0.9 ml cell suspensions were diluted to 3 ml with fresh medium, centrifuged, washed with fresh medium, and finally re-suspended in fresh medium (2 ml). 100 μl of this new 2 ml of cells suspended in fresh medium was then used to determine the cell concentration. This cell concentration is defined as ‘the cell concentration at Day 0 after ultrasound treatment’ (Ci). Cell suspensions were then kept at 37° C. in a 5% CO2 incubator for a total of 10 days.

Over the 10 day period, cells had to be spun down occasionally and re-suspended in fresh medium to replenish the nutrients necessary for a healthy cell population. 100 μl aliquots of the original cell suspension were used to measure the cell concentration both before (Cfinal) and following (Cinitial) re-suspension in fresh medium. This procedure, although necessary, results in a slight underestimation in the expected number of cells on any given day, when compared to the original cell concentration, Ci. To account for this small, but significant underestimation, we calculated the actual number of cells that would have been observed had we not been periodically extracting small aliquots for the detection of cell numbers by the Coulter counter. This was done by calculating a ‘reproduction ratio’, determined by dividing Cfinal by Cinitial that was measured one or two days earlier and comparing this ratio to (Ci), to give a ‘real cell population’. It should be noted that this calculation is done simply as a matter of convenience and that the reproduction ability of the treated samples was compared to control samples that were treated in the same way over the day period, as shown in FIG. 5. Verification of this method is given by the fact that the cells of the control samples double approximately every day (FIG. 5), as expected for HL-60 cells under the conditions of incubation in the current study.

Ultrasound Exposure: unless otherwise stated, the cell suspensions (1 ml) were sonicated in an ultrasonic field in 13×100 mm disposable, autoclaved pyrex tubes (Corning Inc., Corning, N.Y.) exposed to air and fixed in the center of a sonication bath operating at 1.057 MHz (L3 Communications, ELAC-Nautik GmbH, transducer model number 74 051 8052; Cesar generator model number 7500 18003). Sonolysis of a suspension of activated charcoal (1 ml) produced no bands, which indicates the absence of any visible standing wave in the 1 ml sample solution. For experiments with cells, the electrical output of the ultrasound transducer was typically set to 10 W. We have previously characterized the spatially averaged power in the sonicated bath solution under these conditions to be 0.6 Watts/cm2 (Sostaric, J. Z. and Riesz, P. J. 2002), and this calorimetrically determined power input increased linearly as a function of the generator power, from 10 to 60 W (Sostaric, J. Z. and Riesz, P. J. 2002). In the current study, the generator power was quoted as the ultrasound intensity. However, the generator power can be compared to the calorimetrically determined power by referring to the earlier study, where a diagram of the experimental set-up is also available (Sostaric, J. Z. and Riesz, P. J. 2002). The temperature of the coupling water was 25° C. Cell experiments were completed within 5 to 10 minutes of adding the glucopyranosides to the cell suspensions, and each data point represents an average ±SEM, where n=5 to 8. It was found that 15 minute exposures of HL-60 cells to MGP, HGP, HepGP and OGP at the highest concentrations used in this study had no detrimental effect on the reproduction rate of the cells over the course of 120 hours. However, 15 minutes exposure of the cells to DGP resulted in immediate lysis of a large population of cells, as confirmed by Coulter counter and trypan blue staining. For this reason, we only studied the effects of MGP, HGP, HepGP and OGP on the ultrasound induced cytolysis of HL-60 cells. OGP has been used for the non-cytolytic extraction of membrane proteins, where various cells have been exposed to approximately 7 mM to 30 mM concentrations of OGP for up to 30 minutes (Jolly, C. L. et al. 2001; Lazo, J. S, and Quinn, D. E. 1980; Legrue, S. J. et al. 1982), with no cytolytic effects observed. The current study was conducted with OGP concentrations of 3 mM or less and for exposure times of up to 10 minutes and based on previous studies, this surfactant would not be expected to be effective at extracting a significant amount of membrane proteins under the conditions of the current study (Lazo, J. S, and Quinn, D. E. 1980; Legrue, S. J. et al. 1982).

Mechanical Fragility Test: the effect of glucopyranosides on the mechanical fragility of the cells was determined by inducing mechanical shear stress to a cell suspension. This involved placing 10 mL of HL-60 cells in suspension in 125 ml sized screw capped conical flasks containing 10 mL of borosilicate solid-glass beads (Sigma-Aldrich, mean particle diameter of 3 mm), similar to the methods described elsewhere (Carstensen, E. L. et al. 1993; Miller, M. W. et al. 2003). The samples were then shaken in a Burrell wrist action shaker (model number 75, Burrell Scientific, Pittsburgh, Pa.) at 50% power for a duration of 30 minutes. Using this method, up to eight sample solutions could be run at one time. By simultaneously shaking 8 cell suspension samples, it was determined that the position of the samples in the Burrell shaker did not have a significant effect on the percentage of cells that were mechanically lysed (30±2%). In samples containing glucopyranosides, the surfactant was dissolved in 1 ml of DPBS and added to 9 ml of the cell suspension.

Electron Spin Resonance (i.e., ESR or EPR) Measurements: cell suspensions (1 ml) with or without HGP (5 mM) were sonicated in the presence of DBNBS (3 mg/ml), which is effective at spin trapping carbon-centered radicals. Prior to sonolysis, the sample solution containing DBNBS was placed in the pyrex tube and sealed from the atmosphere using a “suba seal” (supplied by Aldrich). The sample was bubbled with argon gas through a needle for 5 minutes. The needle was raised to just above the sample solution, allowing argon gas to pass over the top of the cell suspension during sonolysis (1.057 MHz, p=60 W for 15 seconds). Purging the suspension with argon removes oxygen, thereby avoiding the formation of organic peroxyl radicals that cannot be spin trapped by DBNBS. Immediately following sonolysis, the sample was transferred into an ESR flat quartz cell. The ESR spectra were recorded on a Varian E-9X-band spectrometer with 100 kHz modulation frequency. The typical instrument settings were: modulation amplitude 1 G, time constant 0.128 s, scan speed 0.83 G s⁻¹.

The percentage of cytolysis of HL-60 cells was determined by measurement of the cell size distribution using a Coulter multisizer following sonolysis at 1057 Hz (FIG. 1). We have confirmed the validity of this technique for studying the effects of sonolysis in our ultrasound system by the trypan blue exclusion assay. The mean size of HL-60 cells was determined from the Coulter counter results prior to sonolysis and was approximately 650 μm3 (FIG. 1a) which equates to a mean cell diameter of 13 μm.

Following sonolysis of 1 ml cell suspensions at 1.057 MHz, the number of particles around the original size distribution of HL-60 cells decreased, while a simultaneous increase in much smaller particle sizes (≈100 μm3) was observed, which indicates that the original cells (mean particle volume of 650 μm3) had undergone cytolysis. An extreme example of this is shown in FIG. 1b, where sonolysis was conducted under conditions where almost all of the cells had undergone immediate cytolysis. Ultrasound induced cytolysis was eliminated with the addition of HGP (5 mM) to the cell suspensions just prior to sonolysis (FIG. 1c). Note that the cell size distribution in FIG. 1c looks similar to that shown for untreated, healthy cells (FIG. 1a).

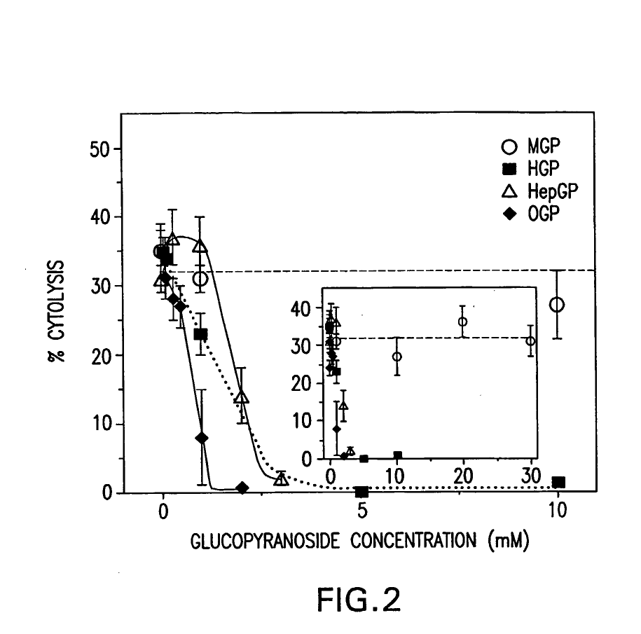

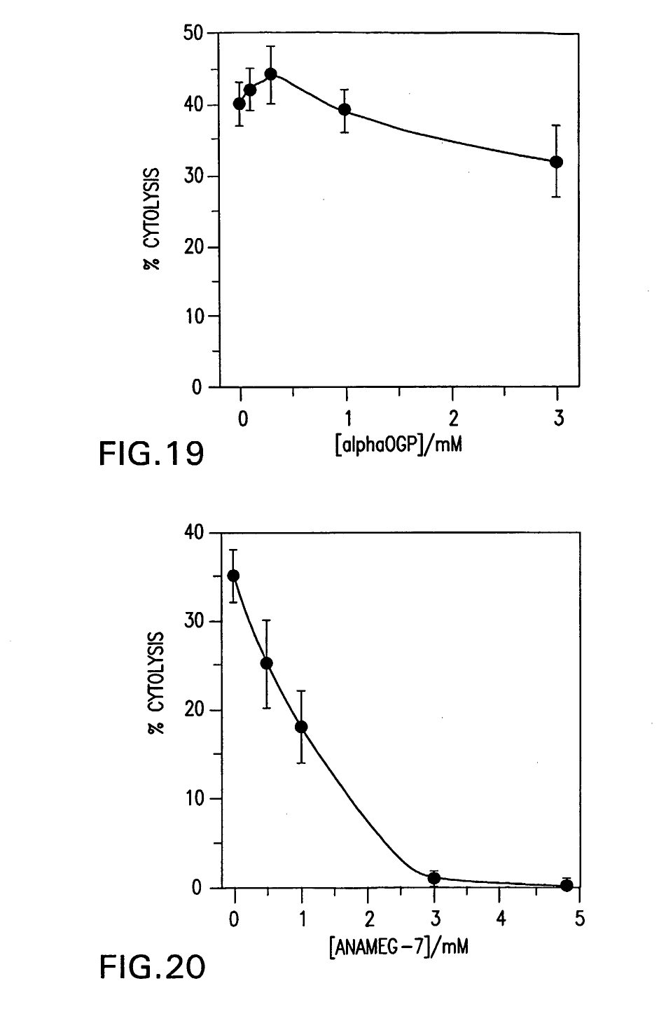

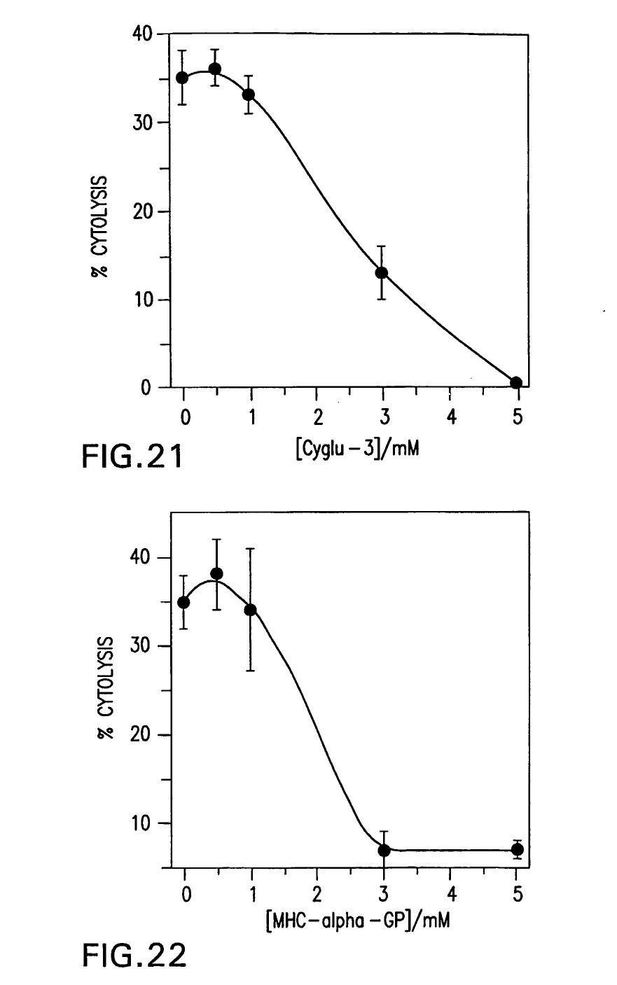

The effect of the concentration of MGP, HGP, HepGP, OGP, alphaOGP, ANAMEG-7, Cyglu-3, and MHC-alpha-GP on the protection of HL-60 cells from immediate cytolysis is shown in FIGS. 2, 19, 20, 21, and 22. The conditions of sonolysis for these experiments were such that approximately 35-40% cytolysis was observed immediately after sonolysis in the absence of any specific additives. For the n-alkyl glucopyranosides shown in FIG. 2, increasing n-alkyl chain length resulted in a more pronounced protection effect, with OGP completely protecting cells at a bulk solution concentration of only 2 mM. MGP, the non-surface active derivative had no effect on cytolysis in the concentration range studied (0 to 30 mM; FIG. 2 insert). AlphaOGP, which is the α-anomer of OGP, demonstrated a very slight protective effect up to 3 mM (FIG. 19). However, ANAMEG-7 (Anatrace, Maume, Ohio) (FIG. 20) and MHC-alpha-GP (FIG. 22), which are also α-D-glucopyranosides, were completely protective at 3 mM. Also demonstrated was complete sonoprotection with CYGLU-3 (Anatrace, Maume, Ohio) at 5 mM (FIG. 21). Coulter counter results which showed that 100% protection occurred following sonolysis were confirmed by trypan blue staining, where only healthy cells were observed at similar concentrations to the untreated controls.

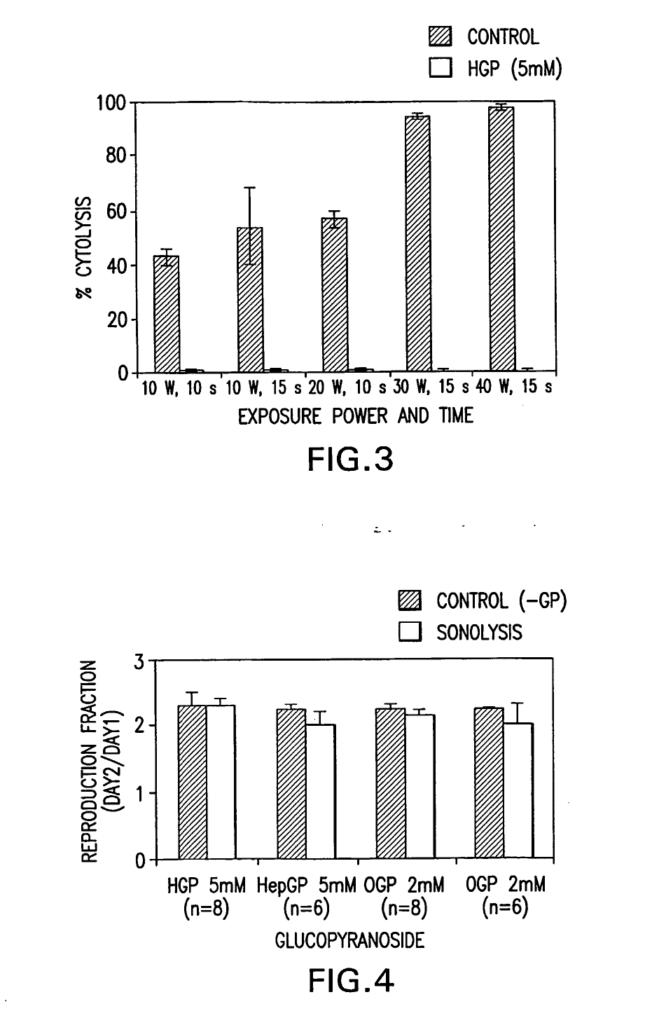

An experiment was conducted to show the effectiveness of HGP (5 mM) in protecting cells from sonolysis under a range of ultrasound intensities and exposure times, as shown in FIG. 3. In this case, it was shown that HGP could protect cells from cytolysis even during extreme conditions of sonolysis where almost 100% of the cell population had undergone cytolysis in the absence of HGP (5 mM).

This dramatic protection effect was confirmed through a series of experiments that studied the reproductive viability of cells following sonolysis in the presence of varying concentrations of HGP, HepGP and OGP over the period of 24 hours, as shown in FIG. 4. Within the experimental error, all of the treated samples continue to reproduce at the same rate as untreated control samples.

Further confirmation of this effect was obtained by conducting an extensive survey of the reproductive capability of cells treated with ultrasound under relatively extreme conditions, but protected from cytolysis by HGP (5 mM), as shown in FIG. 5. Over a period of 10 days, it is clear that the treated cells continue to reproduce at a rate comparable to that of untreated control cells.

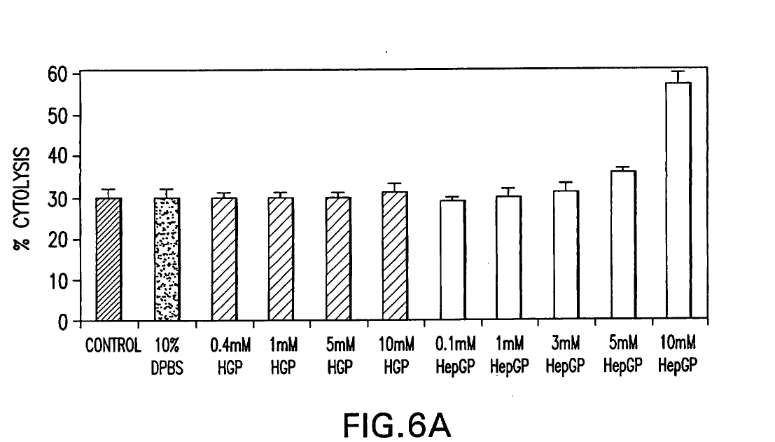

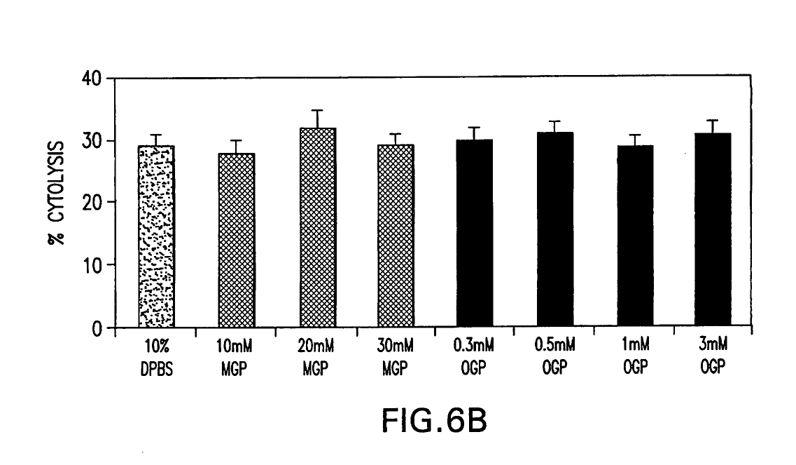

Cavitation induced shear stress is believed to be powerful enough to result in immediate cytolysis. Therefore, it was necessary to test whether the provided surfactants could stabilize cells against the effects of mechanical induced shear stress. FIG. 6 shows that none of the glucopyranosides tested, i.e., HGP (1 mM, 5 mM, 10 mM), HepGP (0.1 mM, 1 mM, 3 mM, 5 mM, 10 mM), MGP (10 mM, 20 mM, 30 mM), or OGP (0.3 mM, 0.5 mM, 1 mM, 3 mM), protected the HL-60 cells from mechanical induced cytolysis. In fact, relatively high concentrations the HepGP surfactant resulted in a significant destabilization of the cell membrane to mechanical shear stress.

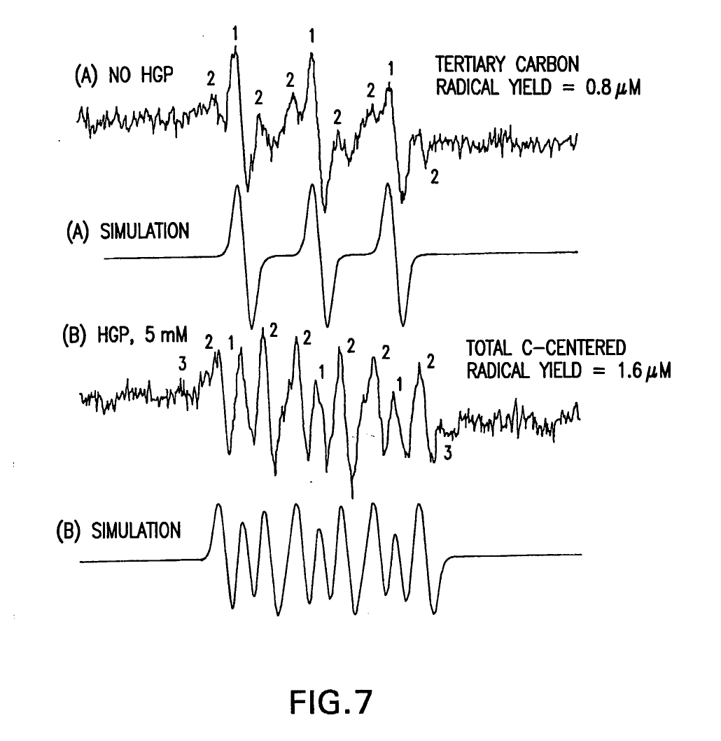

Protection of cells could occur through dampening of the cavitation process by the surfactants. In order to determine whether the surfactants could affect the inertial cavitation process in the cell suspension, we used the technique of spin trapping with DBNBS and electron spin resonance in order to determine the extent of carbon-centered radical formation in the cell suspension in the presence and absence of HGP (5 mM), as shown in FIG. 7. These experiments were done under argon gas, in order to avoid competition reactions between DBNBS and oxygen for carbon-centered radicals. In the absence of HGP, sonolysis of the cell suspension yielded mainly tertiary (R3-.C) carbon-centered radicals with a nitrogen coupling constant of aN =1.5 mT. A very small contribution from secondary (R2-.CH) carbon-centered radicals is also observed. From a simulation of the majority tertiary carbon-centered radical component, a carbon-centered radical yield of 0.8 μM was determined. The addition of HGP (5 mM) to the cell suspension prior to sonolysis yielded an ESR spectrum consisting of both tertiary and secondary carbon-centered radicals with a very small primary (R—.CH2) component. From a simulation of the mainly tertiary and secondary carbon-centered radical components of the ESR spectrum, a total carbon-centered radical yield of 1.6 μM was determined. This is two times higher than the carbon-centered radical yield observed following sonolysis of the cell suspensions in the absence of HGP.

### Example 2

### Effect of Ultrasound Frequency on Sonoprotection by n-alkyl-glucopyranosides

Chemicals: Dulbeco's phosphate buffered saline (DPBS, pH=7.4) was obtained from Biofluids. Methyl β-D-Glucopyranoside (MGP) was obtained from Sigma-Aldrich, hexyl β-D-Glucopyranoside (HGP, ≧98%), heptyl β-D-Glucopyranoside (HepGP, >98%) and octyl β-D-Glucopyranoside (OGP, ≧99%) were obtained from Fluka.

Cells: HL-60 myeloid leukemia cells (American Type Culture Collection) were grown in a suspension of RPMI 1640 medium (GIBCO, Gaithersburg, Md.) containing 10% calf serum. The population of HL-60 cells doubled every 23±1 hr (hour ±SEM) when incubated at 37° C. in a CO₂ (5%) containing atmosphere. Cells were harvested, re-suspended in fresh RPMI medium and kept at 25° C. until the start of the experiment (typically less than 1 hr). The cell concentration was kept constant in all experiments (≈5×10⁵ cells/ml) because of the possible effect of cell concentration on ultrasonically induced cell lysis (Brayman, A. A. et al. 1996). The fraction of intact cells before and after ultrasound was determined using a Coulter multisizer (model IIe) connected to a sampling stand (model IIa). The number of intact cells was determined by counting the total number of particles under the bell shaped curve (e.g., FIG. 1a) before and following sonolysis. The cytolysis percentage was determined by subtracting the number of intact cells following sonolysis from the number of intact cells before sonolysis. This value was divided by the number of intact cells before sonolysis and multiplied by 100 to obtain the cytolysis percentage value.

Ultrasound Exposure: unless otherwise stated, the cell suspensions (1 ml) were sonicated in an ultrasonic field in 13×100 mm disposable, autoclaved pyrex tubes (Corning Inc., Corning, N.Y.) exposed to air and fixed in the center of a sonication bath (L3 Communications, ELAC-Nautik GmbH; Cesar generator model number 7500 18003) operating at frequencies of 1057 or 354 kHz (USW51-052 type, model number 74-051-8052) or 614 kHz (USW51-051 type, model number 74-051-8051). 42 kHz sonolysis was conducted in a similar way but using a Branson ultrasound bath (model number 1510). Sonolysis of a suspension of activated charcoal (1 ml) produced no bands, which indicates the absence of any visible standing wave in the 1 ml sample solution. The electrical output of the ultrasound transducer (1057/354 and 614 kHz) was typically set to 10 W. We have previously characterized the spatially averaged power in the sonicated bath solution under these conditions to be 0.6 Watts/cm² (Sostaric, J. Z. and Riesz, P. J. 2002), and this calorimetrically determined power input increased linearly as a function of the generator power, from 10 to 60 W (Sostaric, J. Z. and Riesz, P. J. 2002). In the current study, the generator power was quoted as the ultrasound intensity. However, the generator power can be compared to the calorimetrically determined power by referring to the earlier study, where a diagram of the experimental set-up is also available (Sostaric, J. Z. and Riesz, P. J. 2002), which is incorporated by reference herein for its teaching of the protocol of the present method. At 42 kHz, a transducer was used to decrease the power of the bath to 50% of its original value. The temperature of the coupling water at all frequencies was 25° C. Cell experiments were completed within 5 to 10 minutes of adding the glucopyranosides to the cell suspensions, and each data point represents an average ±SEM, where n=5 to 8. It was found that 15 minute exposures of HL-60 cells to MGP, HGP, HepGP and OGP at the highest concentrations used in this study had no detrimental effect on the reproduction rate of the cells over the course of 120 hours. OGP has been used for the non-cytolytic extraction of membrane proteins, where various cells have been exposed to approximately 7 mM to 30 mM concentrations of OGP for up to 30 minutes (Jolly, C. L. et al. 2001; Lazo, J. S, and Quinn, D. E. 1980; Legrue, S J. et al. 1982), with no cytolytic effects observed. The current study was conducted with OGP concentrations of 3 mM or less and for exposure times of up to 10 minutes and based on previous studies, this surfactant would not be expected to be effective at extracting a significant amount of membrane proteins under the conditions of the current study (Lazo, J. S, and Quinn, D. E. 1980; Legrue, S J. et al. 1982).

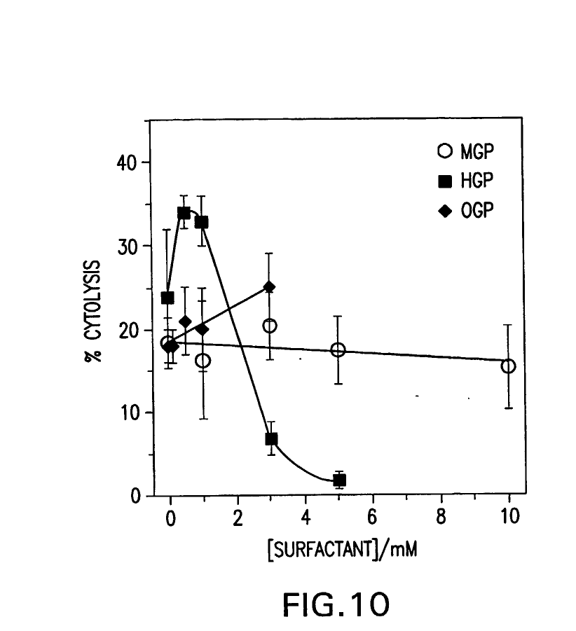

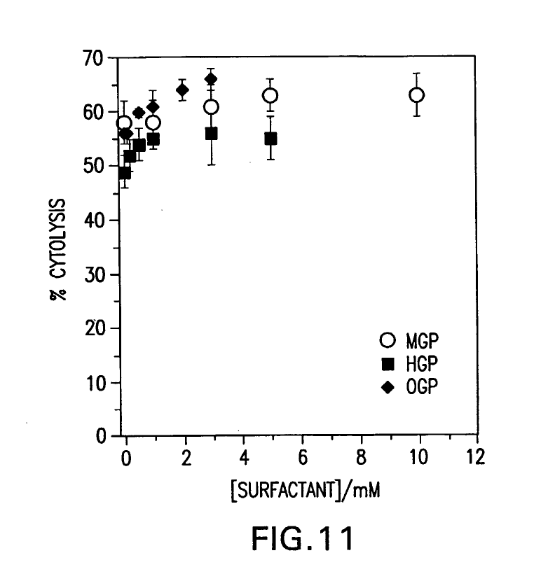

The percentage of cytolysis of HL-60 cells was determined by measurement of the cell size distribution using a Coulter multisizer. This method correlates well with percentage cytolysis measured using the Trypan blue exclusion assay immediately following sonolysis and is explained in detail elsewhere (Miyoshi, N. et al. 2003). Sonolysis of cell suspensions at all frequencies resulted in a certain percentage of cells undergoing cytolysis immediately during ultrasound exposure. This immediate ultrasound induced cytolysis (i.e., % cytolysis) is represented in FIGS. 2, 10-12 at a concentration of zero.

The addition of HGP, HepGP or OGP to the cell suspensions just prior to sonolysis at 1 MHz resulted in a concentration dependent decrease in the percentage of cytolysis, as shown in FIG. 2. Approximately 100% protection from ultrasound induced cytolysis was observed at concentrations of 2 mM (OGP), 3 mM (HepGP) and 5 mM (HGP). It is interesting to note that the concentration of glucopyranoside required to completely protect cells followed the order of the n-alkyl chain lengths of the glucopyranosides, with the longest n-alkyl chain possessing surfactant (OGP) being most effective at protecting cells from ultrasound induced cytolysis. Furthermore, MGP, the non-surface active derivative has no effect on percentage cytolysis at 1 MHz, even up to a concentration of 30 mM. Thus, protection of cells by glucopyranosides from 1 MHz ultrasound is not only dependent on the concentration of glucopyranosides, but also on the surfactant properties of these solutes.

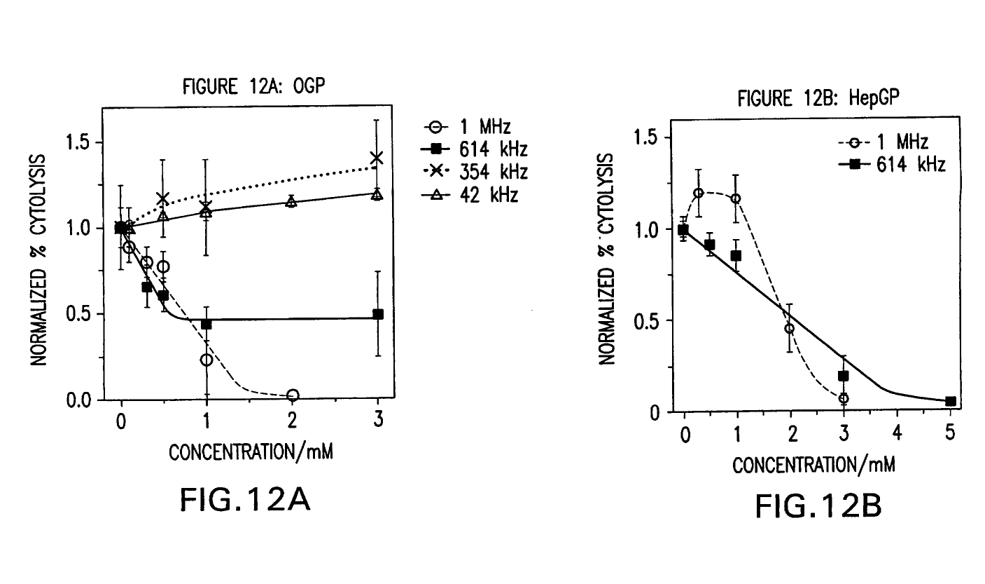

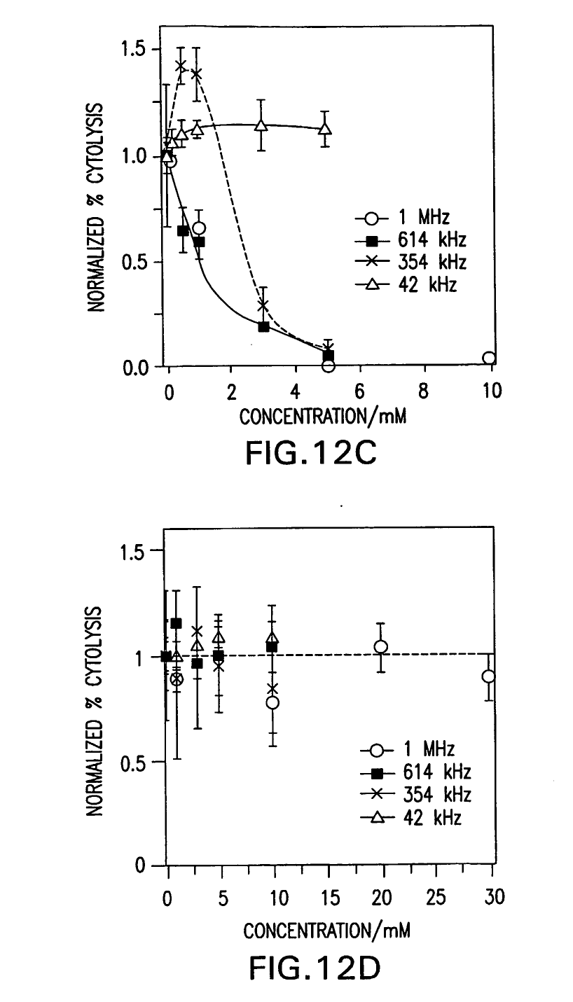

When the frequency of sonolysis is decreased from 1 MHz down to 42 kHz (see FIGS. 2, 9-12) there is a transition for HGP, HepGP and OGP from protection of HL-60 cells (at 1 MHz) to a very small sonosensitization of HL-60 cells at 42 kHz. MGP, however, had no effect on percentage cytolysis, irrespective of its concentration (in the 10 to 30 mM range) or the frequency of sonolysis. In order to gain an appreciation for the effect of ultrasound frequency on the ability of each glucopyranoside to protect HL-60 cells from ultrasound, the percentage cytolysis was normalized to a value of 1 at a concentration of zero and graphed as a function of concentration for each glucopyranoside, at the four different frequencies (FIG. 12a-12d). Normalization of the cytolysis percentage was accomplished by dividing % cytolysis observed at all concentrations of a particular glucopyranoside by % cytolysis observed at a concentration of zero.

Comparing the effect of each surfactant on ultrasound induced cytolysis at the different frequencies shows that: FIG. 12a, OGP fully protects cells from cytolysis at 1 MHz, however at 614 kHz it can only protect 50% of the cell population. When the frequency is decreased to 354 kHz or 42 kHz, OGP acts as a weak sonosensitizer, thereby increasing the % cytolysis. FIG. 12b, HepGP fully protects cells from cytolysis at 1 MHz and 614 kHz. FIG. 12c, HGP fully protects cells at 1 MHz, 614 kHz and 354 kHz, but not at 42 kHz. FIG. 12d, MGP effectively has no effect on ultrasound induced cytolysis at any ultrasound frequency.

There two noticeable trends in the effect of the surface active glucopyranosides (i.e., OGP, HepGP and HGP) on % cytolysis as the frequency is decreased from 1 MHz to 42 kHz. First, as the frequency of sonolysis is decreased, the glucopyranosides become less effective at protecting cells from ultrasound induced cytolysis. Secondly, the ability of the longest n-alkyl chain possessing glucopyranoside (OGP) to protect cells from ultrasound induced cytolysis is most affected by the frequency of sonolysis, compared to HepGP and HGP.

### Example 3

### Maltopyranoside and Thiogalactopyranoside Solutes as Sonoprotectants

Chemicals: Dulbeco's phosphate buffered saline (DPBS, pH=7.4) was obtained from Biofluids. Hexyl-β-D maltopyranoside (HMP), n-octyl-β-D-maltopyranoside (OMP), 2-propyl-1-pentyl-β-D-maltopyranoside (PPMP), n-octyl-β-D-thioglucopyranoside (OTGP), and Isopropyl-β-D-thiogalactopyranoside (IPTGalP) were obtained from Anatrace, Inc., Maumee, Ohio, USA.

Cells: HL-60 myeloid leukemia cells (American Type Culture Collection) were grown in a suspension of RPMI 1640 medium (GIBCO, Gaithersburg, Md.) containing 10% calf serum. The population of HL-60 cells doubled every 23±1 hr (hour ±SEM) when incubated at 37° C. in a CO₂ (5%) containing atmosphere. Cells were harvested, re-suspended in fresh RPMI medium and kept at 25° C. until the start of the experiment (typically less than 1 hr). The cell concentration was kept constant in all experiments (≈5×10⁵ cells/ml) because of the possible effect of cell concentration on ultrasonically induced cell lysis (Brayman, A. A. et al. 1996). The fraction of intact cells before and after ultrasound was determined using a Coulter multisizer (model IIe) connected to a sampling stand (model IIa). The number of intact cells was determined by counting the total number of particles under the bell shaped curve (e.g., FIG. 1a) before and following sonolysis. The cytolysis percentage was determined by subtracting the number of intact cells following sonolysis from the number of intact cells before sonolysis. This value was divided by the number of intact cells before sonolysis and multiplied by 100 to obtain the cytolysis percentage value.

Ultrasound Exposure: unless otherwise stated, the cell suspensions (1 ml) were sonicated in an ultrasonic field in 13×100 mm disposable, autoclaved pyrex tubes (Corning Inc., Corning, N.Y.) exposed to air and fixed in the center of a sonication bath (L3 Communications, ELAC-Nautik GmbH; Cesar generator model number 7500 18003) operating at frequencies of 1057 or 354 kHz (model number xx) or 614 kHz (model number xx). 42 kHz sonolysis was conducted in a similar way but using a Branson ultrasound bath (model number xx). Sonolysis of a suspension of activated charcoal (1 ml) produced no bands, which indicates the absence of any visible standing wave in the 1 ml sample solution. The electrical output of the ultrasound transducer (1057/354 and 614 kHz) was typically set to 10 W. We have previously characterized the spatially averaged power in the sonicated bath solution under these conditions to be 0.6 Watts/cm² (Sostaric, J. Z. and Riesz, P. J. 2002), and this calorimetrically determined power input increased linearly as a function of the generator power, from 10 to 60 W (Sostaric, J. Z. and Riesz, P. J. 2002). In the current study, the generator power was quoted as the ultrasound intensity. However, the generator power can be compared to the calorimetrically determined power by referring to the earlier study, where a diagram of the experimental set-up is also available (Sostaric, J. Z. and Riesz, P. J. 2002). At 42 kHz, a transducer was used to decrease the power of the bath to 50% of its original value. The temperature of the coupling water at all frequencies was 25° C. Cell experiments were completed within 5 to 10 minutes of adding the glucopyranosides to the cell suspensions, and each data point represents an average ±SEM, where n=5 to 8. It was found that 15 minute exposures of HL-60 cells to MGP, HGP, HepGP and OGP at the highest concentrations used in this study had no detrimental effect on the reproduction rate of the cells over the course of 120 hours. OGP has been used for the non-cytolytic extraction of membrane proteins, where various cells have been exposed to approximately 7 mM to 30 mM concentrations of OGP for up to 30 minutes (Jolly, C. L. et al. 2001; Lazo, J. S, and Quinn, D. E. 1980; Legrue, S J. et al. 1982), with no cytolytic effects observed. The current study was conducted with OGP concentrations of 3 mM or less and for exposure times of up to 10 minutes and based on previous studies, this surfactant would not be expected to be effective at extracting a significant amount of membrane proteins under the conditions of the current study (Lazo, J. S, and Quinn, D. E. 1980; Legrue, S J. et al. 1982).

The percentage of cytolysis of HL-60 cells was determined by measurement of the cell size distribution using a Coulter multisizer. This method correlates well with percentage cytolysis measured using the Trypan blue exclusion assay immediately following sonolysis and is explained in detail elsewhere (Miyoshi, N. et al. 2003). Sonolysis of cell suspensions at all frequencies resulted in a certain percentage of cells undergoing cytolysis immediately during ultrasound exposure. This immediate ultrasound induced cytolysis (i.e., % cytolysis) is represented at a concentration of zero.

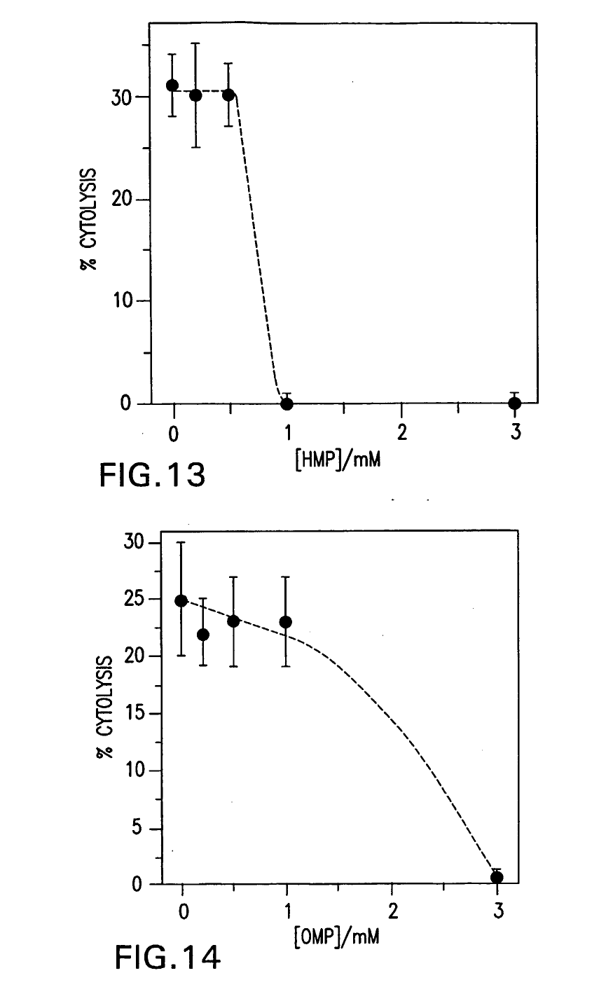

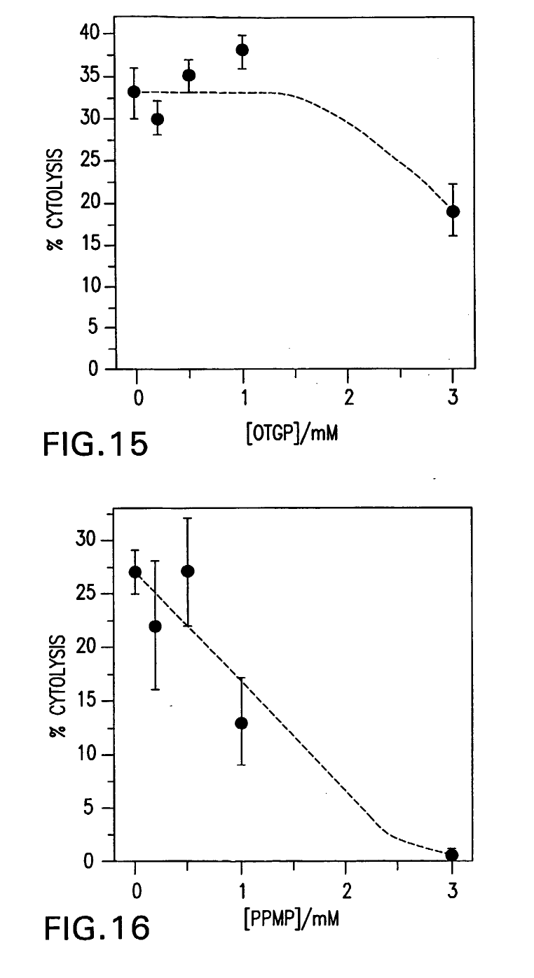

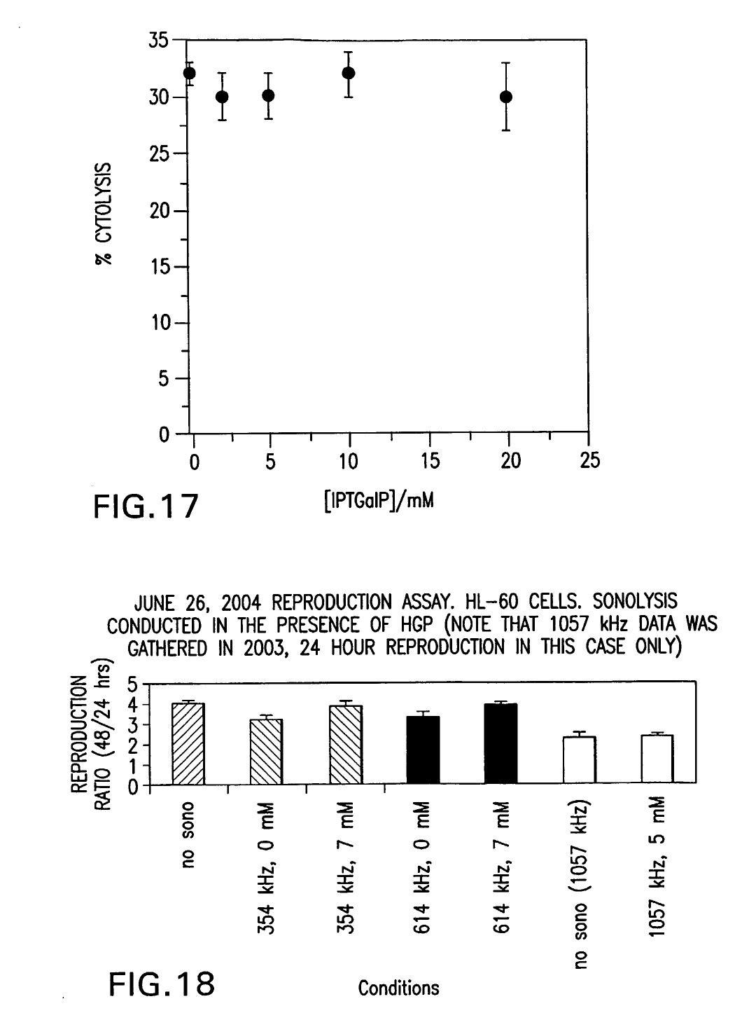

The effect of maltopyranosides (HMP, OMP, PPMP), thioglucopyranosides (OTGP), and thiogalactopyranosides (IPTGalP) on 1 MHz induced cytolysis of HL-60 cells is shown in the FIG. 13-17 (each data point is an average of 4 to 6 runs). FIGS. 13-17 can be compared to the data for HGP shown in FIG. 2.

Glucopyranoside-containing surfactants are not the only type of surfactants that can create this protection effect. The protection effect may be general to any solute with two characteristics: a) the solute possesses surface activity and b) the solute can quench radicals at their source. There are a number of different molecules that could achieve this, not just glucopyranosides, as shown by the example in FIG. 13.

Hexyl-maltopyranoside is more effective at protecting these cells (HL-60) at this frequency (1 MHz) compared to the hexyl-glucopyranoside, i.e., full protection at only 1 mM for HMP, compared to approximately 5 mM for HGP (FIG. 2). This could be due to the fact that the head group of the molecule possesses two sugar entities that can ‘quench’ cytotoxic radicals more effectively than HGP, which possesses only one sugar entity.

### Example 4

### Effect of Sonoprotectants on Long Term Cell Survival

FIG. 18 shows the ‘reproduction ratio’, which is a measure of the ability of the surviving cell population to continue reproducing following treatment by ultrasound in the presence or absence of HGP. The reproduction ratio is simply the number of cells present one or two days post treatment divided by the number of cells present on the treatment day. What the graph shows is that the control (please see the “no sono” bar) doubles in number every day. The “354 kHz, 0 mM” and “614 kHz, 0 mM” bars represent cells that have been treated with ultrasound, in the absence of the protective agents. In other words, they represent a percentage of the original cell population that had survived the initial ultrasound treatment (a certain percentage of the population immediately underwent cytolysis). Finally, the “354 kHz, 7 mM” and “614 kHz, 7 mM” data represent 100% of the cells that were protected from immediate cytolysis. The “no sono” and 7 mM (HGP) bars all continue to reproduce at the same rate. However, the bars labeled “0 mM”, representing ultrasound treated cells that had not been protected by HGP reproduce at a significantly slower rate when compared to the “no sono” control or to the two “7 mM” protected samples. This reduction of reproduction rate for the unprotected “0 mM” populations could occur for one of two reasons, a) either the cells reproductive ability has been diminished due to the effects of ultrasound or b) a proportion of the cells that survived the original ultrasound treatment are slowly dying by a longer term biological pathway, for example apoptosis. In conclusion, the data show that the presence of sonoprotectors during sonolysis of cells also offers a longer term protection against the biologically detrimental effects of ultrasound.

### Example 5

### Treatment of Prostate Cancer, Including Localized Prostatic Adenocarcinoma and Benign Prostatic Hyperplasia

The patient is hospitalized the night before treatment and given an enema for colorectal preparation approximately two hours before treatment. Treatment is executed with the patient lying in a right lateral position. The patient must remain immobile during treatment and is therefore given spinal anesthesia prior to treatment. An ultrasonic probe is inserted into the rectum and a beam of ultrasound is focused, transrectally onto the region of the prostate to be treated. Methods for the application of HIFU to the prostate include: 1) 4 MHz, 211 element PZT and piezocomposite cylindrical transrectal phased arrays (Focus Surgery Inc., Indianapolis, Ind.) 2) Catheter-based, directional transuretheral applicator integrated with a cooling balloon (Ross, A. B., et al. Phys. Med. Biol. 49 (2004) 189-204), 3) Sonoblate-200 HIFU device (Focus Surgery, Inc., Indianapolis, Ind., USA) (Uchida, T., et al. Urology, 59(3), 2002, 394-398), and 4) Ablatherm (EDAP TMS S.A., Lyon, France, www.edap-hifu.com).

The ultrasonic probe is covered by an expandable balloon possessing an aqueous coupling medium. Prior to insertion, pharmacologically suitable paste is added to the outside of the balloon, which comes into contact with the rectal wall. The paste contains a concentration of sonoprotectors of between 0.1 to 30 mM, depending on the sonoprotectors being used. The balloon is expanded following insertion, thereby preventing the applicator from coming into contact with the rectal wall and also helps to cool the rectal wall, since liquid is circulated through the balloon during treatment. The paste possessing the sonoprotectors is between the outer wall of the balloon and the rectal wall, thereby protecting the rectal wall from higher intensities of ultrasound in the unfocussed region. Adsorption of the ultrasonic wave in the region of the focal point (i.e., in the prostate) results in an increase in temperature of 85 to 100 degrees celcius, destroying the cells located in the focal point. The focal point is oval shaped with dimensions measuring up to 24 mm height and 2 mm diameter. 400 to 600 shots of ultrasound are generally applied in order to treat a whole tumor or prostate.

Prostate swelling generally occurs, therefore insertion of a catheter into the urethra is generally necessary for 3 to 8 days post treatment for urination. Generally, a tube is inserted into the urethra to prevent stricture of the urethra, as the prostate swells during treatment. Optionally, to avoid any possibility of damage to cells of the urethra during treatment, a sonoprotector filled tube is inserted into the urethra. The tube is porous to the sonoprotectors, thereby allowing the sonoprotectors to diffuse out of the tube and into contact with the cells of the urethra, thereby protecting them from ultrasound induced damage. The sonoprotector solution is a pharmacologically acceptable aqueous solution containing concentrations of sonoprotectors of the order of 0.1 to 30 mM, depending on the sonoprotectors being used and the frequency of sonolysis being employed.

### Example 6

### Acoustic Hemostasis for Treatment of Punctured Blood Vessels

In order to stop the hemorrhage of human blood vessels, without blocking the vessel, HIFU transducers (Sonic Concepts, Woodinville, Wash.) are used at frequencies of 500 kHz to 5 MHz and with spatial average intensities of between 100 W/cm² to 4000 W/cm². For superficial treatment or treatment of open wounds or treatment during surgical procedures, the transducer is equipped with a conical housing possessing an aqueous solution. The tip of the housing has an opening of about 3 mm and is covered by a suitable polymeric membrane, for example mylar (or polyurethane. The cone geometry is such that the focal point is on the membrane, the membrane being in direct contact with the blood vessel (vein or artery) to be treated. Treatment involves 10 to 20 seconds application of HIFU, followed by a determination of whether bleeding had ceased. Sonoprotectors can be applied as a viscous liquid directly to the region of rupture in concentrations of 0.1 to 30 mM prior to treatment. Treatment times would vary between 10 seconds to 3 minutes, depending on, amongst other things, the size of the rupture. This would be sufficient to lead to coagulation of the adventitia and to create a fibrin network surrounding the vessel wall.

If bleeding is not occurring at a critical rate, sonoprotectors can also be administered by IV in encapsulated nano- or micro-sized particles 0.5 to 5 minutes prior to treatment. The micro- or nano-sized particles can further possess functionality which allows them to accumulate at the site of injury. For example, microbubbles with lipid shells can bind to leukocytes by opsonization, whereby a serum complement that is deposited on the surface of the microbubble can bind to a number of different receptors that exist on activated leukocytes at the site of trauma (Springer, T; Ann. Rev. Physiol. 1995, 57, 827-872).

Hemostasis in the liver can be further enhanced by the presence of a contrast agent. Optison® at a concentration of 0.09 ml/kg to 0.3 ml/kg in saline is injected into a mesenteric vein that drains into the portal vein. The contrast agent enters the liver lobe which can be determined by a significant increase in the liver echogenecity using ultrasound imaging. Time after injection would be of the order of 0.5 to 5 minutes. In a similar way, the liver is exposed to sonoprotectors, either through direct injection of the sonoprotectors into the mesenteric vein at concentrations of between 0.1 to 30 mM, or in encapsulated form in polymeric microspheres at much higher concentrations, up to approximately 100 mM, dissolved in a suitable biological medium encapsulated by the microsphere or other pharmaceutically acceptable delivery device as described at the beginning of the section. The HIFU device can operate at frequencies from approximately 750 kHz to 5 MHz as a single element unit. Mutlielement units can also be employed for focusing and in situ ultrasound imaging of the treatment. For example, a 750 kHz to 5 MHz inner element with an outer element of lower frequency, approximately 100 to 500 kHz can be employed. Superposition of one or two frequencies in this way allows for a greater range of ultrasound effects to be created. Typical ultrasound intensities would be of the range from 100 to 5000 W/cm². The ultrasound applicator can be scanned either manually or automatically over the region of bleeding. Ultrasound administration can be either continuous, short bursts of 1 to 5 seconds to prevent overheating, or can be applied continuously in an automatic pulsed mode, which automatically controls the length of time that the applicator remains on and off, with on:off ratios on the ms time scale. In such a regime, on and off times could be of the order of 1 ms to 1000 ms, with on:off ratios in the range from 1:1000 to 1:1.

The addition of contrast agents, such as Albunex, are extremely valuable for the in vivo diagnostic ultrasound detection of vessel or artery injury (rupture) following trauma. However, it has been shown that the presence of contrast agents in the blood increases hemolysis through a cavitation process during application of HIFU to in vitro blood samples. The following describes the use of sonoprotectors for hemostasis.

Systemic concentrations of Albunex can be below the manufacturer's maximum of 0.3 ml/kg of body weight. Assuming a body weight of 70 kg and a blood volume of 5 L, the maximum allowable Albunex concentration, assuming uniform distribution in the body following several minutes of administration can be estimated as 4.2 μl of contrast agent per ml of blood. Sonoprotectors can be incorporated into the core of polymeric microspheres or other delivery agents, or introduced as a mixture with contrast agents. As HIFU is applied to the ruptured vessel or artery, the contrast agent promotes cavitation, but at the same time ruptures and promotes release of the sonoprotectors from the polymeric microspheres, in the region being treated. Thus HIFU acts by heating the tissue and creating coagulation at the site of vessel or artery rupture, while the sonoprotectors protect blood and surrounding tissue from cavitation induced hemolysis and cytolysis respectively. Concentrations of sonoprotectors employed would be of the order of 1 to 100 mM in the encapsulated form, which would decrease substantially following ultrasound induced rupture of the microspheres in the trauma region, down to concentrations that would be pharmaceutically acceptable and where sonoprotecting properties of the solutes would still be present.

### Example 7

### Protection of Surrounding Healthy Tissue During Ultrasound Mediated Thermal Ablation of Uterine Leiomyoma and Other Uterine Cancers, Uterine Fibroids and Control of Uterine Bleeding

The transducer employed can be similar in nature to a transvaginal transducer being developed by Vaizy, S. and co-workers (Chan, A. H., et al. Fertility And Sterility 82(3), 2004), which is an image-guided HIFU device that operates at between 1 to 4 MHz frequency. The applicator is covered by a balloon possessing a degassed aqueous solution that circulates through the balloon to provide cooling and prevent direct contact between the transducer and the vaginal wall. Sonoprotector (0.1-30 mM) is applied externally on the balloon wall in the form of a paste. This ensures direct acoustic coupling between the balloon and the vaginal wall and at the same time protects cells on the vaginal wall from ultrasound mediated damage. Real time imaging can be achieved using a hand held ultrasound system integrated into the ultrasound application device (SonoSite, Bothell, Wash., www.sonosite.com).

Prior to treatment, the patient is sedated with their abdomen facing upward on the operating table. A balloon catheter is inserted into the urethra and the bladder filled with a minimum of 200 mL of saline to improve transabdominal ultrasound imaging. Once the position of the uterus and pelvic structures are determined using the ultrasound imaging probe, a dilator is used to insert a tube of sufficient size into the vagina to aid the insertion of the HIFU applicator, which is covered by the deflated balloon, which in turn is covered by ample amount of paste possessing sonoprotectors. The balloon is filled with aqueous solution (50 to 200 mL) and the applicator is positioned so that the focus is on the region of the uterus to be treated. Sonication is conducted at 1 to 10 second intervals with 20 to 100 W of acoustic power, or a spatial average temporal average of between 1000 to 4000 W/cm², which would be sufficient to cause tissue necrosis and allow an echoic spot to appear on the ultrasound image. Using computer guidance and ultrasound imaging, successive spots can be placed next to each other to treat a larger volume. Sonoprotectors are supplied to the uterus through IV injection in encapsulated form, as described, at encapsulated concentrations of 1 to 100 mM, 1 to 30 minutes prior to treatment. As ultrasound ruptures the polymeric microspheres in the uterus, sonoprotectors are released, thereby protecting all tissue from cavitation induced damage, while allowing thermal ablation of the treatment area through direct adsorption of the ultrasound wave.

Alternate treatment methods could be employed for ultrasound application, not requiring transvaginal application, as described by Hynynen and co-workers (Tempany, C. M. C., et al. Radiology, 266 (3), 2003, 897-905). In that case, ultrasound is applied with a clinical MR imaging—compatible focused ultrasound system (ExAblate 2000; In-Sightec-TxSonics, Haifa, Israel, www.insightec.com). A focused piezoelectric transducer array operating at a frequency of between 1.0 and 1.5 MHz generates the ultrasonic field. The array is positioned in a water tank. A computer controls the location of the focal spot and the coagulated tissue volume. A thin plastic membrane window covers the water tank and allows the ultrasound to penetrate into the patients pelvis. A flexible gel pad contours to the shape of the patient and covers the thin plastic membrane. Degassed water is poured onto the gel to ensure good acoustic coupling between the patient and the ultrasound transducer. Again, sonoprotectors are delivered in encapsulated form to the uterus.

### Example 8

### Protection of Surrounding Tissue During Ultrasound Mediated Treatment of Breast, Liver and Kidney Cancer

An ultrasound exposure system, such as the Exablate 2000 (InSightec Co, www.insightec.com) or Ultrasound Model-JC Tumor Therapy System (Chongquin HAIFU Technology Company, China, http://www.haifu.com.cn/en/index.asp, can be used to treat tumors of the breast, kidney and liver. These instruments operate in the region of 0.8 to 1.8 MHz, which is the region of maximum sonoprotection properties of the sonoprotectors.

The microspheres are formed by any pharmaceutically acceptable method, such as those described by Kennith Suslick and co-workers (U.S. Pat. Nos. 5,498,421; 5,635,207; 5,639,473; 5,650,156; and 5,665,382). Polymeric microspheres consist of a pharmaceutically viable solution comprising the sonoprotectors in concentrations of between 1 to 100 mM, depending on the sonoprotectors being used. The particle size is 3 to 4.5 microns, the particle concentration is 5-8×10⁸ particles/mL, with a total dose for any one subject not to exceed 15 mL. Intravenous injection is continuous and does not exceed 1 mL per second. Approximately 0.5 to 5 minutes following administration, treatment can begin. Microspheres that enter the focal region of the ultrasound beam rupture due to the physical action of the ultrasonic wave on the microsphere. This results in the sudden release of sonoprotectors in and in the region of the focal point. The initial relatively high concentration of sonoprotectors encapsulated within the microspheres is rapidly diluted in the region of treatment to non-toxic levels where the sonoprotectors still retain their sonoprotecting ability. Thus, the final concentration of sonoprotectors in the region to be treated are instantaneously in the order of 0.1 to 30 mM, depending on the sonoprotecting agent being employed.

It should be noted that the microbubbles can not be completely filled with solution possessing sonoprotector, since a gas space is required so that the bubbles can rupture and release the sonoprotecting agents. Another method is to have a mixture of microbubbles that are filled with varying amounts of sonoprotecting solution (from empty bubbles to fully-filled bubbles) that are used together. In this way, the bubbles possessing less of the sonoprotectors solution violently oscillate and rupture, creating physical forces in the vicinity of partially and fully filled microbubbles that cause them to rupture also. Alternatively, microbubbles can be brought to rupture by application of other techniques including the application of electric or magnetic fields, heat or light to particles susceptible to rupture under such conditions.

To ensure that the microbubbles reach sufficient concentrations at the site to be treated by ultrasound, specific targeting methods can be employed. For example, the intrinsic properties of the microbubble shell or monoclonal antibodies and other ligands can be conjugated to the microbubble shell so that the microbubbles recognize antigens that are expressed in regions of diseased tissue only, for example, tumor cells. As another example of how microbubbles an be directed to specific sites, the microbubble shell can be made to possess a relatively large electrostatic charge. Externally applied electric fields can be used to direct the particles to the site to be treated, and/or to trap and retain a relatively large concentration of microbubbles in the treatment region.

Prior to administration, an intravenous access is created, for example, in a peripheral vein with a 20 gauge angiocatheter. The polymeric microbubbles, which are treated with care, so as to prevent their breakage, are suspended in a suitable sterile liquid. The particle suspension, which should be at room temperature, is administered through an IV line or a short sized extension tubing at a steady rate, from 0.5 to 1 mL/second. An ultrasound scan of the region to be treated is used to observe the build up of microbubbles, which will have some contrast in the ultrasonic field.

Sonoprotectors are administered 1 to 30 minutes prior to HIFU treatment to protect healthy cells in the breast, kidney and liver from cavitation induced damage, while allowing thermal ablation of tumors to occur through adsorption of the ultrasonic wave in the focal point. The microbubbles can possess certain functionality which allows for their accumulation in the region of the tumor. For example, microbubbles of 10 to 200 nm diameter can preferentially accumulate within a broad range of tumor types, most probably because of a compromise in the endothelial integrity of the microvasculature of tumors. This is observed for nanosized liposomes, which are accumulated in tumors in this way (Papahadjopoulos, D, et al. Proc Natl. Acad. Sci. 1991; 88(24):11460-4).

### Example 9

### Protection of Tissue During Low and High Frequency Sonophoresis

Sonophoresis can be used to deliver macromolecules that otherwise cannot penetrate the skin such as, for example, insulin, mannitol, heparin, morphine, caffeine, lignocaine, DNA (for gene therapy of the skin). During sonophoresis, ultrasound is transferred from the transducer to the skin through a coupling medium, due to the high acoustic impedance of air. The coupling medium can be an oil, water-oil emulsion, aqueous gel or ointment. The ultrasound applicator can operate at either high (3 to 10 MHz), medium (0.7 to 3 MHz) or low (16 to 700 kHz) frequency. Ultrasound intensities can lie in the range of 0.1 to 50 W/cm², depending on the size of the molecules to be transported, thus the size of the pores required to allow their passage through the skin.

The SonoPrep® skin permeation device (Sontra Medical Corp., www.sontra.com), which operates at 55 kHz, can be employed for sonophoresis. Prior to treatment, the subject's skin is supplied a gel, paste, ointment, emulsion or similar substance which comprises sonoprotectors in a concentration of 0.1 to 100 mM, depending on the sonoprotectors being used. Drug delivery (gene transfection) can be conducted in situ by incorporating the drug (vector/naked DNA) within the gel, paste, ointment, emulsion, etc. . . . . Alternatively, once the skin has been sonoporated, a patch containing a pharmaceutically acceptable or required dose of the particular drug is applied to the region of sonophoresis. Pores in the skin remain open long enough to allow for diffusion of the drug through the stratum corneum (the outer layer of the skin). As sonophoresis dramatically decreases the lag time necessary for a topical anaesthetic, for example EMLA® cream (AstraZeneca; http://www.astrazeneca.com), to take effect, sonoprotectors can be mixed into said cream at concentrations of 0.1 to 100 mM to prevent cavitation induced damage to cells of the skin.

Furthermore, 1 to 30 minutes prior to treatment, encapsulated sonoprotecting agents can be intravenously administered to the patient. As the skin is treated with ultrasound, the polymeric microspheres possessing the sonoprotectors will rupture in the lower layers of the skin, thereby protecting the lower layers of the skin, blood vessels and capillaries from cavitation induced damage and cytolysis.

Alternatively, the coupling medium can comprise 0.1 to 100 mM sonoprotectors. Higher frequency sound waves (1 to 5 MHz) are adsorbed by and result in the sonophoresis of the upper layers of skin. This allows the sonoprotectors to slowly diffuse into lower layers of skin. As this occurs, gradually lower frequencies of ultrasound can be employed to create cavitation in the lower layers of skin and allow penetration of sonoprotectors into still lower regions of skin, which the sonoprotecting agents protect against cavitation induced cell damage and cytolysis at the subsequently applied lower ultrasound frequencies.

### Example 10

### Protection of Cells in the Ultrasound Mediated Treatment of Brain Tumor, Vascular Thrombosis and Disruption of the Blood Brain Barrier

Ultrasound frequency with lower and upper limits of 40 kHz to 2 MHz, and more appropriately, 100 kHz to 1 MHz range are used in transcranial applications. Application of the ultrasound wave is monitored in situ using a 2 MHz or higher diagnostic ultrasound unit to avoid the formation of standing waves, which can cause higher energy deposition of the wave well outside of the focal region. Standing wave formation can be avoided by using a pulsed ultrasound regime. Ultrasound intensity at the thrombus lies in the region of 0.1 W/cm² to 35 W/cm² temporal average for thrombolysis to be achieved, and more specifically from 0.1 W/cm² to 10 W/cm². It can be expected that intensities in the lower range at <1 W/cm² could be effectively employed for successful thrombolysis of a clot in the presence of ultrasound contrast agents for the treatment of vascular thrombosis and in the presence of pharmaceutical thrombolytic agents such as tissue plasminogen activator (t-PA), urokinase (UK) and alteplase specifically for the treatment of stroke. Treatment duration is of the order of 15 minutes to up to a maximum of 4 hours, although 15 minute to 1 hour treatment times is most common.

Microbubbles or nanosized bubbles for ultrasound treatment are prepared with encapsulation of the sonoprotectors at concentrations of between 0.1 to 100 mM as described above. The microbubbles are suspended in a pharmaceutically acceptable solution and administered by IV for 1 to 15 minutes prior to ultrasound exposure, at a maximum concentration of between 0.05 mL to 0.9 mL per kg of body weight.

Furthermore, in the case of thrombus destruction, the shell of the micro- or nano-bubbles can have ligands conjugated on the surface which recognize platelet and/or fibrin components of that clot, thereby accumulating more readily in the region to be treated by ultrasound. As an example, MRZ-408 particles (ImaRx Corp., Tucson, Ariz., USA) target platelet glycoprotein IIb/IIIa receptor on the surface of activated platelets. Application of ultrasound on the site of the thrombus, or the region where BBB disruption is to occur, follows. For ultrasound induced thrombolysis, treatment can also be conducted in the presence of a thrombolytic agent, such as t-PA and administered at a pharmaceutically acceptable dose before ultrasound treatment. Rupture of polymeric microspheres can be brought about by the ultrasonic wave if at high enough intensity. Alternatively, release of sonoprotectors from the polymeric particles an be achieved through other forms of energy, including electric or magnetic stimuli. Once the sonoprotectors are released in the region of ultrasound treatment, they diffuse through the tissue to protect the whole region from ultrasound mediated damage and cytolysis, while allowing the physical effects of ultrasound to enhance thrombolysis or to transiently permeate the blood brain barrier without damaging cells or creating cytolysis or causing hemorrhage.

Currently, for treatment of thrombi in other regions of the body, for example, myocardial infarction or deep vein thrombosis, catheter mediated treatments are being employed. Although ultrasound treatment could potentially replace this type of invasive treatment method, ultrasound can also be used in conjunction with catheter treatment. First, anti-thrombolytic drugs, heparin followed by warfarin, can stabilize the thrombus following IV injection. The catheter is then used to remove the thrombus. The catheter can further deliver sonoprotectors to the region of the thrombus at a concentration of between 0.1 mM to 30 mM. At this stage, ultrasound would be applied to the region of the thrombus to enhance blood flow during treatment through physical effects, while the surrounding tissue is protected from cavitation induced damage. Most recently, catheters possessing miniaturized ultrasound transducers are being developed for intra arterial delivery of ultrasound and thrombolytic agents. The transducers operate in the range of 100 kHz to 300 kHz and can also be used for delivery of sonoprotectors to the region of the thrombus, prior to ultrasound exposure.

### Example 11

### Protection of Cells and Surrounding Tissue During Gene Therapy and Drug Delivery to Cells In Vitro and In Vivo (Sonoporation)

Ultrasound exposure is conducted by a flat plate type transducer, either in direct contact with the cells suspended in a cell culture medium or in contact with a bath full of coupling medium that transmits the wave to the cells which are suspended in or attached to either a stationary or rotating tube, plate, conical flask, cell culture flask, dish or other container suspended in the coupling medium. The coupling medium can, for example, be an aqueous solution that has been presonicated. Presonication allows the solution to be degassed and reach a constant, equilibrium temperature that can be controlled by an external water jacket surrounding the coupling medium. Degassing of the coupling medium is important for reproducibility of the results and reduction of cavitation bubble formation in the coupling medium, which can affect the passage of the waves through the coupling medium. Temperature control is important to ensure reproducible cavitation conditions. The length of time required for the degassing procedure depends on, amongst other variables, the volume of coupling liquid to be used, the ultrasound exposure conditions and the temperature of exposure. As a guide, for exposure conditions in the frequency range of 354 to 1057 kHz, over a broad range of intensities from the onset of cavitation to the maximum possible cavitation effect, in a temperature range from 10 degrees celcius to 30 degrees celcius and for a coupling liquid that consists of Milli-Q filtered water, an exposure time of approximately 10 to 15 minutes is sufficient to reach steady-state conditions in the coupling medium. The exposure temperature can lie anywhere in the range from above freezing to 40 degrees celcius, but for most cell lines, a range of 20 degrees celcius to 37 degree celcius is sufficient. One purpose of sonicating at lower temperatures, for example 20 degrees celcius, is that lower evaporation rates of water from the cell culture medium ensures that the volume of medium does not change significantly during sonolysis. Secondly, lower temperatures tend to, in general but not in all instances, promote acoustic cavitation effects, including sonochemistry and sonoluminescence. This may not be the case under certain ultrasound exposure conditions, especially above 1 MHz, in very specific ultrasound intensity ranges, namely towards the low intensity end. Thus, an alternate exposure system could consist of a transducer immersed into a bath of much larger volume, or a transducer irradiating ultrasonic waves into a bath of much larger volume (i.e., a 1 to 8 gallon tank of water). The container possessing the cells can also be immersed into the bath and ultrasound passed through the container possessing the cells and to one end of the bath which possesses an absorber to prevent reflection of the wave and formation of a standing wave in the system. In this way, the ultrasonic wave is focused onto the sample to be irradiated. Furthermore, the container in this case can be constructed in the form of a Teflon, metal or suitable plastic cylindrical housing which is closed off at each end by extremely thin Mylar® windows to allow the ultrasonic wave to pass through the chamber with little absorption or reflection of the ultrasonic wave. For bath water of such large volume, the water is degassed before treatment using a typical degassing system.

The frequency of sonolysis employed is in the range from 20 kHz to 2 MHz. Ultrasound intensities lie in the range from 0.01 W/cm² to 100 W/cm², and it is preferable to work at intensities that are above the cavitation threshold either in the presence or absence of ultrasound contrast agents. Exposure times are of the order of 5 seconds to 5 minutes and are either continuous or pulsed mode. Ultrasound contrast agents significantly lower the threshold for cavitation, i.e., they are cavitation promoters. One would have to determine in any given system whether it would be advantageous to add contrast agents to the system or not. Typically, a relatively small proportion of sonoporated cells would result for a relatively large proportion of cytolysis. However, to avoid cytolysis, sonoprotectors are added to the cells in the container just prior to sonolysis. The concentrations of sonoprotectors to be employed would lie in the range from 0.25 mM to 30 mM to achieve a degree of protection to the cells from cytolysis, while allowing the physical effects of ultrasound, or ultrasound plus microbubbles, to sonoporate the cells. Following sonoporation, the cells are incubated for 5 to 60 minutes under the appropriate incubation conditions for the given cell line, with typical conditions including a 5% CO₂ atmosphere and a temperature of 37 degrees celcius. They are be rinsed with fresh medium to remove the sonoprotectors and the drug, naked DNA, DNA vector or other material that was sonoporated and is still remaining in the cell culture medium.

For sonoporation of cells located deep inside the body, systems such as the ExAblate 2000; In-Sightec-TxSonics, Haifa, Israel, www.insightec.com) or the Ultrasound Model-JC Tumor Therapy System (Chongquin HAIFU Technology Company, China, http://www.haifu.com.cn/en/index.asp) can be used to focus ultrasound energy onto the site to be treated. For a more superficial treatment, for example of skin or muscle tissue, non-focusing transducers can be applied in combination with an appropriate coupling substance. The ultrasound conditions can be in the range of 20 kHz to 2 MHz frequency and ultrasound intensities of the order of 0.1 W/cm² to 50 W/cm². Delivery of sonoprotectors encapsulated in microspheres at concentrations of 0.1 to 100 mM for deeper tissues could be achieved through IV injection in conjunction with an echo contrast agent to aid in the sonoporation process. The mixture of sonoprotecting microspheres and echo contrast agents can be administered at maximum concentrations of up to 0.3 ml/kg of body weight for a microbubble solution containing, for example Optison or Abunex contrast agent bubbles. Treatment can begin once a high enough concentration of microbubbles reaches the treatment site, as determined by continuous ultrasound scanning of the region to be treated. Ultrasound ruptures the sonoprotecting microbubbles either directly (for partically filled microbubbles) or through the indirect action of ultrasound and collapse of gas filled microbubble/echo contrast agents in close vicinity to sonoprotectors solution filled microbubbles.

Alternative forms of microbubble rupture can also be employed, as discussed above. Plasmid or naked DNA or the drug of interest could also be delivered in conjunction with microspheres or liposomes that encapsulate the genetic material or the drug and release it at the treatment site. For the treatment of superficial tissues, for example the skin and muscle tissue, transfection material, drugs and sonoprotectors could also be administered directly to the tissue by direct injection into the tissue.

### Example 12

### Protection of Endothelial Cells During Ultrasound Treatment for Phacoemulsification or Sonophoresis

The cornea is a biological barrier which allows only a small amount (5 to 10%) of a drug to pass into the anterior of the eye. 2 to 3 fold enhancement can be achieved with ultrasound in the mid range of between about 400 to 900 kHz frequency, with transient endothelial cell damage caused by cavitation effects (Zderic, V; et al. J. Ultrasound Med., 2004, 23, 1349-1359). However, the use of sonoprotectors could protect endothelial cells from damage, and also allow higher intensities of ultrasound to be employed, thereby enhancing the sonophoresis effect considerably, while protecting endothelial cells from cavitation induced damage at higher ultrasound intensities.

The ultrasound apparatus, e.g., UZT-1.03 O (Electrical and Medical Apparatus, Moscow, Russia), consists of a flat transducer with a diameter of 0.5 to 3 cm, which is ultimately determined based on the diameter of the cornea. After administration of a local anaesthetic, an eye-cup is positioned onto the eye of the patient. The end of the eye cup is made of a suitable material that can be positioned under the eyelids to make a temporary seal between the surface of the eye and the cup. To the cup is added a pharmaceutically acceptable aqueous solution possessing sonoprotectors in the concentration range of 0.1 to 100 mM and the drug to be delivered to the eye. This solution could be a balanced salt ophthalmic solution typically used in the clinic. The transducer is placed a short distance (0.1 to 1 cm) above the cornea and ultrasound is supplied to the whole cornea, since the transducer is chosen so that it is of similar diameter to the diameter of the patient's cornea. Ultrasound conditions would lie in the frequency range of 20 kHz to 2 MHz, with optimal frequencies being in the range of 100 kHz to 800 kHz. Ultrasound intensities would lie in the range of 0.1 to 5 W/cm², depending on the frequency to be employed (higher intensities would be expected for higher ultrasound frequencies, since the cavitation threshold increases with increasing ultrasound frequency). Treatment regimes can consist of either pulsed ultrasound bursts or continuous ultrasound application for total times of between 0.5 to 10 minutes. For example, lower treatment times would be used for combinations of low frequency but high ultrasound intensity treatment. Following ultrasound treatment, the eye cup remains on the eye of the patient for 1 to 5 minutes. During this time, the coupling solution possessing the sonoprotectors and the drug can be decanted while fresh solution possessing drug only is added to the eye cup, allowing drug to diffuse through any pores created in the extracellular space between the sonoprotected endothelial cells.

Furthermore, sonoprotectors can be used to prevent corneal damage that can arise with the use of high energy ultrasound during phacoemulsification surgery. Following application of a general anesthetic, the eye is cleansed with topical povidone iodine. Following insertion of a lid speculum, a corneal incision is made in the superotemporal corneal quadrange. A phacoemulsification probe (for example Series Ten Thousand Phacoemulsification system, Alcon Surgical, Fort Worth, Tex., USA) set at 50 to 80% power and 15 to 25 ml/min of irrigation is introduced into the anterior chamber without contacting the cornea, lens or other ocular structures. The probe is activated in the center of the anterior chamber. Time of phacoemulsification can be from 1 to 10 minutes. The phacoemulsificator should be controlled by a variable voltage control, allowing the probe to operate in a 1:1 pulsed mode to avoid overheating. Sonoprotectors are added to the irrigation solution at a concentration of 0.1 to 100 mM prior to treatment, thereby protecting cells from ultrasound induced cytolysis.

### Example 13

### Protection of Plant, Animal or Microbial Cells in Ultrasound Bioreactors

The enhanced metabolic productivity of microorganisms, plant and animal cells (the “living organism”) in bioreactors can result in more efficient biotechnological processes. Examples of organisms that could be used in bioreactors are Anabaena flos-aquae, a cyanobacterium, Selenastrum capricornutum, Lactobacillus delbrueckii cells, hybridoma culture, Petunia hybrida plant cell, Panax ginseng suspended cells, Lithospermum erythrorhizon cells, Micromonospora echinospora, filamentous fungal cells such as Rhizopus arrhizus NRRL 1526, and CHO cells. Controlled sonication, i.e., relatively low power sonication, is being employed in an attempt to enhance bioreactor processes with minimal damage to the living organism. Ultrasound can enhance diffusion within and outside a cell and thereby enhance rates of reactions and metabolic yields. Alternatively, in certain bioprocesses, the system can be pre-sonicated before addition of the living organism, for example to break a sludge into smaller particles or to decompose larger molecules to smaller molecules that can be more easily biodegraded. An example of this has been shown for the biodegradation of distillery wastewater (Preeti C., et al. Ultrasonics Sonochem., 11 (2004) 197-203). Such processes would be greatly enhanced and more time and energy efficient if ultrasound is used at higher intensities in the presence of the living organism in the bioreactor. The problem is that using higher ultrasound intensities and ultrasound exposure times has a simultaneous adverse effect on retention of the living organisms since, for example, higher ultrasound intensities of longer exposure times results in unacceptable levels of cell disruption and cytolysis. Thus, described is a general method for using low or high power sonication, for enhancing bioreactor processes while protecting the living organisms from ultrasound mediated cytolysis.

Bioreactor design depends on the biotechnological process of interest and on the scale of the process. The reactor system can be a static system or a continuous flow through system (Yusuf C. Trends in Biotechnology, 21(2),2003), disclosed herein by reference in its entirety for its teaching of sonobioreactor designs. Sonolysis can be conducted in the frequency range of 20 kHz to 1 MHz, with optimal frequencies in the range of 100 kHz to 500 kHz. The latter frequency range is a good balance between cavitation production ability, compared to frequencies of more than 500 kHz, resulting in, for example, better mass transfer. Furthermore, the 100 kHz to 500 kHz range is also a region where sonoprotectors are expected to have better protecting ability, compared to frequencies of less than 100 kHz. In a static bioreactor system, sonoprotector can be added directly to the bioreaction prior to sonolysis, at a final concentration of 0.1 mM to 100 mM, depending on the sonoprotector to be employed. Concentrations could even be ten times higher, i.e. 1 mM to 1 M, depending on the particular system. For example, bioreactors which possess a large amount of small particulate matter with an amorphous surface can adsorb much of the added sonoprotector from the solution. Alternatively, certain organisms can digest the sonoprotectors. To counter this, larger concentrations of sonoprotectors need to be added to ensure enough availability of sonoprotectors in the bulk solution and at the interface of cavitation bubbles, to act as sonoprotecting agents.

Addition of sonoprotectors to the bioreactor can best be achieved by adding the sonoprotector in the form of a stock solution at higher concentration. For example, the stock solution can be an aqueous solution of sonoprotector in the concentration range of 1 to 100 mM. The volume of stock sonoprotector solution to be added to the bioreactor would thus equal one tenth the volume of the bioreactor process, making a final concentration of 0.1 to 1000 mM of sonoprotector in the bioreactor. Again, higher concentration stock solutions can be used for sonobioreactors possessing high amounts of amorphous particles, for example. Depending on the size of the bioreactor, the reaction system is circulated to ensure a homogeneous distribution of sonoprotectors throughout the system. In a flow through bioreactor, stock sonoprotector solution is continually added to the bioreaction at a rate determined so that the instantaneous steady-state concentration of sonoprotectors remains in the concentration range of 0.1-100 mM, or higher.

In this way, this method opens up two new possibilities for ultrasound bioreactors. First, rather than pre-treating a reaction system and then adding the living organism for biodegradation, ultrasound could now be applied in situ, while the living organism will be protected from ultrasound cavitation induced cytolysis. Secondly, in systems where the living organism is already present, sonoprotectors protect the living organism from damage thereby enhancing the biological process and also allowing for a higher intensity of ultrasound to be employed to further enhance the process, without creating substantial cytolysis or destruction of living organisms. Times of ultrasound exposure, ultrasound intensities, the number of ultrasound transducers and their geometrical layout depend on the bioreaction of interest, and the type of living organism being used. The effect of different ultrasound conditions on various living organisms for bioreactors are known in the art (Yusuf C, Trends in Biotechnology, 21(2), 2003), incorporated herein for its teaching of the effect of ultrasound on living organisms in bioreactors.

### Example 14

### Cell Size

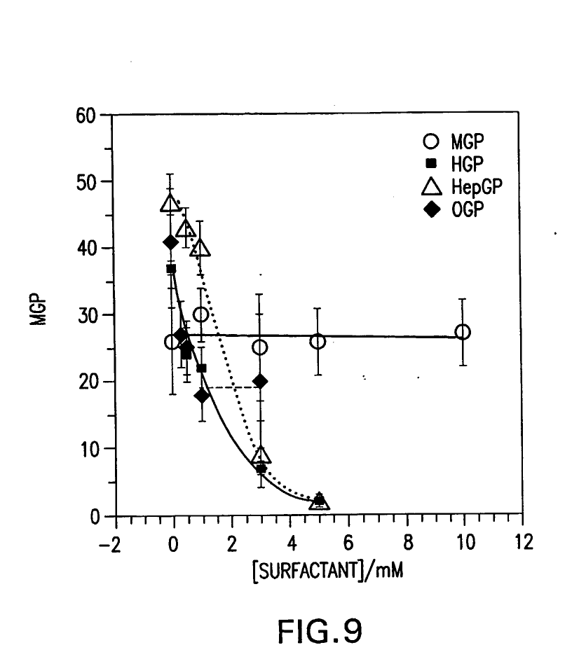

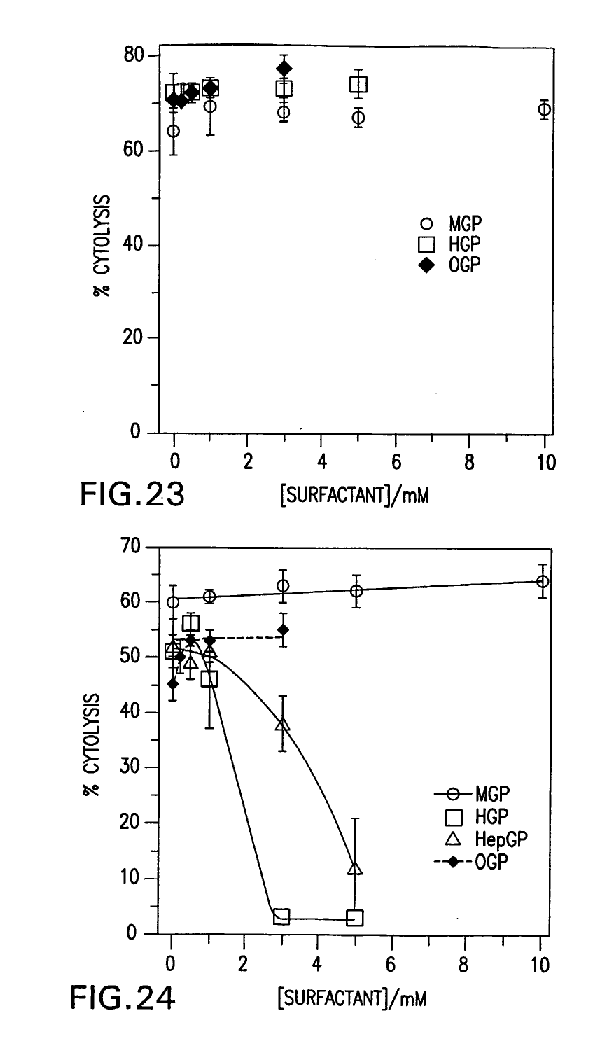

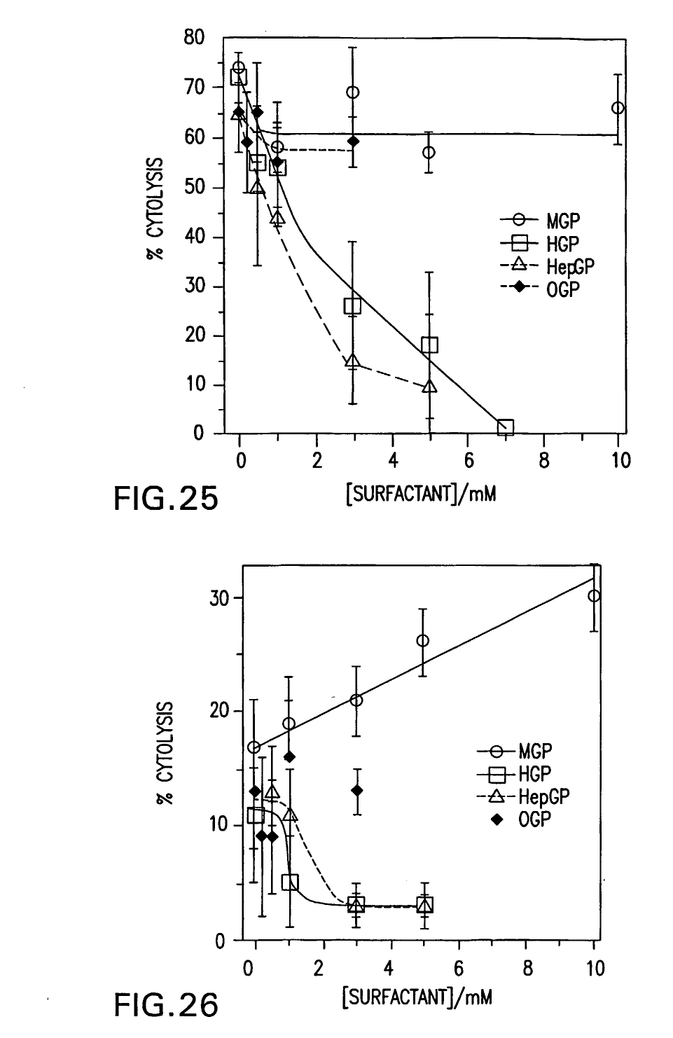

Glucopyranosides can also protect larger sized HL-525 cells from ultrasound induced cytolysis (FIGS. 23-26). As was the case for protection of HL-60 cells (FIGS. 12a to 12d), the protective effect for HL-525 cells was also ultrasound frequency dependent. However, there are some clear differences between protection for HL-525 cells and their smaller, HL-60 counterparts. For example, at a sonolysis frequency of 1 MHz (compare FIG. 2 with FIG. 26) it is clear that OGP and MGP had different protection effects for the two cells. Likewise, at 614 kHz, the protective effect of OGP was much less pronounced for HL-525 cells, compared to HL-60 cells (compare FIG. 9 with FIG. 25). These indicate selectivity in the protective effect of these molecules for different cell lines.

### Example 15

### Mechanical Destruction

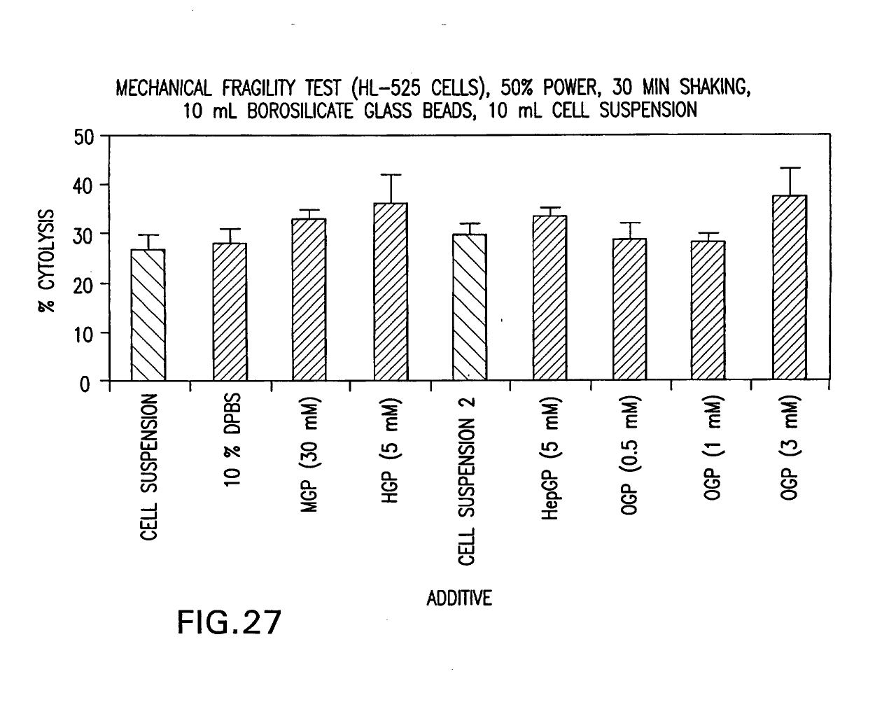

As shown in FIG. 27, HL-525 cells are slightly susceptible to an increased mechanical destruction pathway caused by the presence of glucopyranosides, compared to their HL-60 counterparts, which were generally unaffected (FIG. 6). For example, OGP (3 mM) had a significant effect on enhancing mechanical destruction of HL-525 cells (FIG. 27) but no effect on the mechanical destruction of HL-60 cells (FIG. 6). This may explain, at least in part, why OGP exhibited a smaller protective effect for HL-525 cells at 1 MHz and 614 kHz compared to HL-60 cells.

### Example 16

### Selective Destruction of Diseased Cells in Blood

The use of ultrasound in combination with glucopyranosides and mixtures of glucopyranosides can be used as a treatment for certain diseases of the blood. For example, an excessive leukocyte count in patients with chronic myelogenous leukemia can be controlled with the selective ultrasonic cytolysis of excess leukocytes, without damage to other cellular components of blood. This avoids the use of highly toxic drugs, such as busulfan, which would otherwise be required to control the leukocyte count in such patients with chronic, long term myelogenous leukemia. The patient can undergo a procedure almost identical to that of typical hemodialysis treatment used to remove impurities from the blood of patients who have kidney failure.

The patient can undergo the internal access procedure by either arteriovenous (AV) fistula or AV graft to surgically join an artery and vein under the skin in the arm, or surgically grafting a donor vein respectively. This procedure allows the vascular system to support a blood flow of 250 milliliters per minute required for a typical dialysis treatment. During a typical four hour treatment time, 60 liters of blood recirculates through the dialysis system, which accounts for approximately 10 cycles for the average person. A number of weeks following surgery, the patient can be prepared for their first hemodialysis/ultrasound treatment. A topical anesthetic is applied to the patients skin at the access point. Two needles that are connected to soft tubes that go directly to the dialysis machine are inserted into the artery and vein. The port from the artery leads into the ultrasound unit for blood treatment, prior to entering a typical dialysis machine. Before blood enters the hemodialysis machine, glucopyranosides are injected into the system at the required dose. The steady state concentration of glucopyranosides can be in the range from 0.1 to 5 mM, and various mixtures of glucopyranosides can be employed, so as to maximize the detrimental effects of ultrasound to diseased cells, while protecting healthy cells from cytolysis. Immediately following injection, the blood is treated in a flow through ultrasound unit consisting of an array of ultrasonic transducers operating in a frequency of 20 kHz to 5 MHz and intensities of between 1 to 80 W. The blood then passes into a typical dialysis unit. Initially passing through a pump, anticoagulant is added to the blood to prevent coagulation. The blood passes through a dialyzer where impurities, including the glucopyranosides are removed following contact with the semipermeable membranes of the dialyzer. An air trap just after the dialyzer and detectors throughout the line monitor the pressure in the blood to maintain safety. The second port in the skin then allows for the introduction of treated blood into the vein. Each dialysis treatment can last approximately 1 to 4 hours and can be conducted at times when the patient's leukocyte count has risen to 50,000 cells per cubic millimeter. Treatment can end when the patient's leukocyte count has dropped to just under 10,000 cells per cubic millimeter.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### 1. REFERENCES

Aguilera, J. A.; Newton, G. L.; Fahey, R. C.; Ward, J. F. Thiol Uptake by Chinese-Hamster V79-Cells and Aerobic Radioprotection as a Function of the Net Charge on the Thiol. Radiat. Res. 130:194-204; 1992.
Alegria, A. E.; Lion, Y.; Kondo, T.; Riesz, P. Sonolysis of Aqueous Surfactant Solutions—Probing the Interfacial Region of Cavitation Bubbles by Spin Trapping. J. Phys. Chem. 93:4908-4913; 1989.
Alfassi, Z. B.; Huie, R. E.; Neta, P. Kinetic Studies of Organic Peroxyl Radicals in Aqueous Solutions and Mixed Solvents. In: Alfassi, Z. B., eds. Peroxyl Radicals. West Sussex: John Wiley and Sons Ltd.; 1997:235-281.
Apfel, R. E. Acoustic Cavitation Prediction. J. Acoust. Soc. Am. 69:1624-1633; 1981.
Armour, E. P.; Corry, P. M. Cyto-Toxic Effects of Ultrasound In vitro Dependence on Gas Content, Frequency, Radical Scavengers, and Attachment. Radiat. Res. 89:369-380; 1982.
Bae, Y. H., Okano, T., Husu, R. & Kim, S. W. Thermo-sensitive polymers as on-off switches for drug release. Makromol. Chem. Rapid Commun. 8:481-485 (1987).
Bothe, E.; Schulte-Frohlinde, D.; von Sonntag, C. Radiation Chemistry of Carbohydrates. Part 16. Kinetics of HO2 elimination from peroxyl radicals derived from glucose and polyhydric alcohols. J. Chem. Soc., Perkin Trans. II 416-420; 1977.
Brayman, A. A.; Church, C. C.; Miller, M. W. Re-evaluation of the concept that high cell concentrations “protect” cells in vitro from ultrasonically induced lysis. Ultrasound Med. Biol. 22:497-514; 1996.
Carstensen, E. L.; Kelly, P.; Church, C. C.; Brayman, A. A.; Child, S. Z.; Raeman, C. H.; Schery, L. Lysis of erythrocytes by exposure to CW ultrasound. Ultrasound Med. Biol. 19:147-165; 1993.
Church, C. C.; Flynn, H. G.; Miller, M. W.; Sacks, P. G. The Exposure Vessel as a Factor in Ultrasonically-Induced Mammalian-Cell Lysis 0.2. An Explanation of the Need to Rotate Exposure Tubes. Ultrasound Med. Biol. 8:299-309; 1982.
Crum, L. A. Measurements of the Growth of Air Bubbles by Rectified Diffusion. J. Acoust. Soc. Am. 68:203-211; 1980.
Curiel, L.; Chavrier, F.; Gignoux, B.; Pichardo, S.; Chesnais, S.; Chapelon, J. Y. Experimental evaluation of lesion prediction modelling in the presence of cavitation bubbles: intended for high-intensity focused ultrasound prostate treatment. Med. Biol. Eng. Comput. 42:44-54; 2004.
Didenko, Y. T.; McNamara, W. B.; Suslick, K. S. Hot spot conditions during cavitation in water. J. Am. Chem. Soc. 121:5817-5818; 1999a.
Didenko, Y. T.; McNamara, W. B.; Suslick, K. S. Temperature of multibubble sonoluminescence in water. J. Phys. Chem. A 103:10783-10788; 1999b.
Didenko, Y. T.; Pugach, S. P.; Gordeichuk, T. V. Sonoluminescence spectra of water: Effect of the ultrasonic irradiation power. Opt. Spektrosk. 80:913-919; 1996.
Dooley, D. A.; Sacks, P. G.; Miller, M. W. Production of Thymine Base Damage in Ultrasound-Exposed Emt6 Mouse Mammary Sarcoma-Cells. Radiat. Res. 97:71-86; 1984.
Edelman, E. R., Kost, J., Bobeck, H. & Langer, R. Regulation of drug release from polymer matrices by oscillating magnetic Fields. J. Biomed. Mater. Res. 19:67-83 (1985).
Ellwart, J. W.; Brettel, H.; Kober, L. O. Cell-Membrane Damage by Ultrasound at Different Cell Concentrations. Ultrasound Med. Biol. 14:43-50; 1988.
Fahey, R. C. Protection of DNA by Thiols. Pharmacol. Ther. 39:101-108; 1988.
Ferri, J. K.; Stebe, K. J. Which surfactants reduce surface tension faster? A scaling argument for diffusion-controlled adsorption. Adv. Colloid Interface Sci. 85:61-97; 2000.
Fischel-Ghodsian, F., Brown, L., Mathiowitz, E., Brandenburg, D. & Langer, R. Enzymatically controlled drug delivery. Proc. Natl. Acad. Sci. USA 85:2403-2406 (1988).
Fu, Y.-K.; Kaufman, G. E.; Miller, M. W.; Griffiths, T. D.; Lange, C. S. Modification by cysteamine of ultrasound lethality to Chinese hamster V-79 cells. Radiat. Res. 80:575-580; 1979.
Fyrillas, M. M.; Szeri, A. J. Surfactant dynamics and rectified diffusion of microbubbles. J. Fluid Mech. 311:361-378; 1996.
Grieser, F.; Ashokkumar, M.; Sostaric, J. Z. Sonochemistry and Sonoluminescence in Colloidal Systems. In: Crum, L. A.; Mason, T. J.; Reisse, J. L.; Suslick, K. S., eds. Sonochemistry and Sonoluminescence. Dordrecht: Kluwer Academic Publishers; 1999:345-362.
Hahn, S. M.; Tochner, Z.; Krishna, C. M.; Glass, J.; Wilson, L.; Samuni, A.; Sprague, M.; Venzon, D.; Glatstein, E.; Mitchell, J. B.; Russo, A. Tempol, a Stable Free-Radical, Is a Novel Murine Radiation Protector. Cancer Res. 52:1750-1753; 1992a.
Hahn, S. M.; Wilson, L.; Krishna, C. M.; Liebmann, J.; Degraff, W.; Gamson, J.; Samuni, A.; Venzon, D.; Mitchell, J. B. Identification of Nitroxide Radioprotectors. Radiat. Res. 132:87-93; 1992b.
Halliwell, Barry; Gutteridge, John M. C. Free Radicals in Biology and Medicine. New York: Oxford University Press; 1999.
Harvey, E. N. 61:2392; 1939.
Henglein, A. Sonochemistry—Historical Developments and Modern Aspects. Ultrasonics 25:6-16; 1987.
Hristov, P. K.; Petrov, L. A.; Russanov, E. M. Lipid peroxidation induced by ultrasonication in Ehrlich ascitic tumor cells. Cancer Lett. 121:7-10; 1997.
Inoue, M.; Church, C. C.; Brayman, A.; Miller, M. W.; Malcuit, M. S. Confirmation of the Protective Effect of Cysteamine in Invitro Ultrasound Exposures. Ultrasonics 27:362-369; 1989.
Jolly, C. L.; Bersner, B. M.; Ozser, E.; Holmes, I. H. Non-lytic extraction and characterisation of receptors for multiple strains of retavirus. Arch. Virol. 146:1307-1323; 2001.
Kawai, N.; Iino, M. Molecular damage to membrane proteins induced by ultrasound. Ultrasound Med. Biol. 29:609-614; 2003.
Kennedy, J. E.; ter Haar, G. R.; Cranston, D. High intensity focused ultrasound: surgery of the future? Br. J. Radiol. 76:590-599; 2003.
Kondo, T.; Fukushima, Y.; Kon, H.; Riesz, P. Effect of Shear-Stress and Free-Radicals Induced by Ultrasound on Erythrocytes. Arch. Biochem. Biophys. 269:381-389; 1989.
Kondo, T.; Kano, E. Effect of Free-Radicals Induced by Ultrasonic Cavitation on Cell Killing. Int. J. Radiat. Biol. 54:475-486; 1988.
Kost, J., Leong, K. & Langer, R. Ultrasound-enhanced polymer degradation and release of incorporated substances. Proc. Natl. Acad. Sci. USA 86:7663-7666 (1989).
Kwon, I. C., Bae, Y. H. & Kim, S. W. Electrically erodible polymer gel for controlled release of drugs. Nature 354: 291-293 (1991).
Lagneaux, L.; de Meulenaer, E. C.; Delforge, A.; Dejeneffe, M.; Massy, M.; Moerman, C.; Hannecart, B.; Canivet, Y.; Lepeltier, M. F.; Bron, D. Ultrasonic low-energy treatment: A novel approach to induce apoptosis in human leukemic cells. Exp. Hematol. 30:1293-1301; 2002.
Lazo, J. S.; Quinn, D. E. Solubilization of Pulmonary Angiotensin-Converting Enzyme with 1-O-Normal-Octyl-Beta-D-Glucopyranoside. Anal. Biochem. 102:68-71; 1980.
Legrue, S. J.; Macek, C. M.; Kahan, B. D. Non-Cytolytic Extraction of Murine Tumor-Specific Transplantation Antigens with the Non-Ionic Detergent Octyl-Beta-Deuterium-Glucopyranoside. J. Natl. Cancer Inst. 69:131-136; 1982.
Leighton, T. G. The Acoustic Bubble. London: Academic Press; 1994.
Li, T. L.; Tachibana, K.; Kuroki, M. Gene transfer with echo-enhanced contrast agents: Comparison between Albunex, Optison, and Levovist in mice—Initial results. Radiology 229:423-428; 2003.
Lippitt, B.; McCord, J. M.; Fridovich, I. The sonochemical reduction of cytochrome c and its inhibition by superoxide dismutase. J. Biol. Chem. 247:4688-4690; 1972.
Marmottant, P.; Hilgenfeldt, S. Controlled vesicle deformation and lysis by single oscillating bubbles. Nature 423:153-156; 2003.
Mason, T. J. Sonochemistry: The Uses of Ultrasound in Chemistry. Cambridge: Royal Society of Chemistry; 1990.
Mason, T. J.; Lorimer, J. P. Sonochemistry: Theory, applications and uses of Ultrasound in Chemistry. West Sussex: Ellis Horwood Limited; 1988.
Mathiowitz, E. & Cohen, M. D. Polyamide microcapsules for controlled release. V. Photochemical release. J. Membr. Sci. 40,:67-86 (1989).
McNamara, W. B.; Didenko, Y. T.; Suslick, K. S. Sonoluminescence temperatures during multi-bubble cavitation. Nature 401:772-775; 1999.
Melodelima, D.; Chapelon, J. Y.; Theillere, Y.; Cathignol, D. Combination of thermal and cavitation effects to generate deep lesions with an endocavitary applicator using a plane transducer: Ex vivo studies. Ultrasound Med. Biol. 30:103-111; 2004.
Miller, D. L.; Thomas, R. M.; Frazier, M. E. Ultrasonic Cavitation Indirectly Induces Single-Strand Breaks in DNA of Viable Cells-Invitro by the Action of Residual Hydrogen-Peroxide. Ultrasound Med. Biol. 17:729-735; 1991.
Miller, M. W.; Miller, D. L.; Brayman, A. A. A review of in vitro bioeffects of inertial ultrasonic cavitation from a mechanistic perspective. Ultrasound Med. Biol. 22:1131-1154; 1996.
Miller, M. W.; Miller, W. M.; Battaglia, L. F. Biological and environmental factors affecting ultrasound-induced hemolysis in vitro: 3. Antioxidant (Trolox (R)) inclusion. Ultrasound Med. Biol. 29:103-112; 2003.
Misik, V.; Miyoshi, N.; Riesz, P. Effects of cysteamine and cystamine on the sonochemical accumulation of hydrogen peroxide—Implications for their mechanisms of action in ultrasound-exposed cells. Free Radic. Biol. Med. 26:961-967; 1999.
Misik, V.; Riesz, P. EPR characterization of free radical intermediates formed during ultrasound exposure of cell culture media. Free Radic. Biol. Med. 26:936-943; 1999.
Misik, V.; Riesz, P. Free radical intermediates in sonodynamic therapy. Ann. N.Y. Acad. Sci. 899:335-348; 2000.
Mitchell, J. B.; Degraff, W.; Kaufman, D.; Krishna, M. C.; Samuni, A.; Finkelstein, E.; Ahn, M. S.; Hahn, S. M.; Gamson, J.; Russo, A. Inhibition of Oxygen-Dependent Radiation-Induced Damage by the Nitroxide Superoxide-Dismutase Mimic, Tempol. Arch. Biochem. Biophys. 289:62-70; 1991.
Mitragotri, S.; Kost, J. Low-frequency sonophoresis—A review. Adv. Drug Deliv. Rev. 56:589-601; 2004.
Miyata K, Maruoka S, Nakahara M, Otani S, Nejima R, Samejima T, Amano S. Corneal endothelial cell protection during phacoemulsification: low-versus high-molecular-weight sodium hyaluronate. J Cataract Refract Surg. 28(9):1557-60; 2002.
Miyoshi, N.; Misik, V.; Riesz, P. Sonodynamic toxicity of gallium-porphyrin analogue ATX-70 in human leukemia cells. Radiat. Res. 148:43-47; 1997.
Miyoshi, N.; Sostaric, J. Z.; Riesz, P. Correlation between sonochemistry of surfactant solutions and human leukemia cell killing by ultrasound and porphyrins. Free Radic. Biol. Med. 34:710-719; 2003.
Neppiras, E. A. Acoustic Cavitation. Phys. Rep.-Rev. Sec. Phys. Lett. 61:159-251; 1980.
Neppiras, E. A.; Noltingk, B. E. 64B:1032-1038; 1951.
Newton, G. L.; Aguilera, J. A.; Ward, J. F.; Fahey, R. C. Binding of radioprotective thiols and disulfides in Chinese hamster V79 cell nuclei. Radiat. Res. 146:298-305; 1996.
Noltingk, B. E.; Neppiras, E. A. Cavitation produced by ultrasonics. 63B:674-685; 1950.
Nozaki, T.; Ogawa, R.; Feril, L. B.; Kagiya, G.; Fuse, H.; Kondo, T. Enhancement of ultrasound-mediated gene transfection by membrane modification. J. Gene. Med. 5:1046-1055; 2003.
Sacks, P. G.; Miller, M. W.; Church, C. C. The Exposure Vessel as a Factor in Ultrasonically-Induced Mammalian-Cell Lysis 0.1. A Comparison of Tube and Chamber Systems. Ultrasound Med. Biol. 8:289-298; 1982.
Schuchmann, M. N.; von Sonntag, C. Radiation Chemistry of Carbohydrates. Part 14 Hydroxyl Radical Induced Oxidation of D-glucose in oxygenated aqueous solution. J. Chem. Soc. Perkin Trans. II 1958-1963; 1977.
Siegel, R. A., Falamarzian, M., Firestone, B. A. & Moxley, B. C. pH-controlled release from hydrophobic/polyelectrolyte copolymer hydrogels. J. Control. Release 8:179-182 (1988).
Sostaric, J. Z. Interfacial Effects on Aqueous Sonochemistry and Sonoluminescence. Ph.D. Thesis, The University of Melbourne, 1999. (http://eprints.unimelb.edu.au/archive/00000504/)
Sostaric, J. Z.; CarusoHobson, R. A.; Mulvaney, P.; Grieser, F. Ultrasound-induced formation and dissolution of colloidal CdS. J. Chem. Soc.-Faraday Trans. 93:1791-1795; 1997.
Sostaric, J. Z.; Mulvaney, P.; Grieser, F. Sonochemical Dissolution of Mno2 Colloids. J. Chem. Soc.-Faraday Trans. 91:2843-2846; 1995.
Sostaric, J. Z.; Riesz, P. Adsorption of surfactants at the gas/solution interface of cavitation bubbles: An ultrasound intensity-independent frequency effect in sonochemistry. J. Phys. Chem. B 106:12537-12548; 2002.
Sostaric, J. Z.; Riesz, P. Sonochemistry of surfactants in aqueous solutions: An EPR spin-trapping study. J. Am. Chem. Soc. 123:11010-11019; 2001.
Stottlemyer, T. R.; Apfel, R. E. The effects of surfactant additives on the acoustic and light emissions from a single stable sonoluminescing bubble. J. Acoust. Soc. Am. 102:1418-1423; 1997.
Suslick, K. S. Sonochemistry. Science 247:1439-1445; 1990.
Suslick, K. S. Ultrasound: Its Chemical, Physical and Biological Effects. 1988
Suslick, K. S.; Hammerton, D. A.; Cline, R. E. The Sonochemical Hot-Spot. J. Am. Chem. Soc. 108:5641-5642; 1986.
Takahashi H, Sakamoto A, Takahashi R. Ohmura T, Shimmura S, Ohara K. Free radicals in phacoemulsification and aspiration procedures. Arch Opthalmol. 120(10):1348-52; 2002.
Yasui, K. Effect of surfactants on single-bubble sonoluminescence. Phys. Rev. E 58:4560-4567; 1998.
Young, F. R. Cavitation. London, U.K.: McGraw-Hill; 1989.
Zheng, S. X.; Newton, G. L.; Gonick, G.; Fahey, R. C.; Ward, J. F. Radioprotection of DNA by Thiols—Relationship between the Net Charge on a Thiol and Its Ability to Protect DNA. Radiat. Res. 114:11-27; 1988.

1. A method for protecting cells from ultrasound-mediated cytolysis comprising delivering to the cells a surfactant, wherein the surfactant comprises at least one unit having the formula I 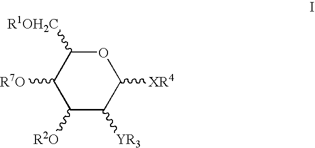
wherein X is oxygen, sulfur, or NR⁵, and
Y is oxygen, sulfur, or NR⁶,
wherein R¹-R⁷ are each, independently, hydrogen, a branched- or straight-chain alkyl group, a substituted or unsubstituted aryl group, an aralkyl group, a cycloalkyl group, an ester group, an aldehyde group, a keto group, an amide group, a residue of a saccharide, or a combination thereof,
or the pharmaceutically-acceptable salt or ester thereof,
wherein at least one of R¹-R⁷ is a hydrophobic group,
wherein the surfactant is not sodium chondroitin sulfate, sodium hyaluronate, or a combination thereof.

2. The method of claim 1,
wherein the surfactant has molecular weight less than 5,000 Da.

3. The method of claim 1, wherein the surfactant has a molecular weight less than 1,000 Da.

4. The method of claim 1, wherein the surfactant comprises less than 10 units having the formula I.

5. The method of claim 1, wherein R⁴ is a hydrophobic group and R¹-R³ and R⁷ are, independently, hydrogen or a residue of a saccharide.

6. The method of claim 1, wherein R⁷ is a hydrophobic group and R¹-R⁴ are, independently, hydrogen or a residue of a saccharide.

7. The method of claim 1, wherein at least one of R¹-R⁴ and R⁷ is hydrogen.

8. The method of claim 1, wherein X and Y are oxygen.

9. The method of claim 1, wherein R¹-R³ are hydrogen.

10. The method of claim 1, wherein R⁷ is hydrogen.

11. The method of claim 1, wherein R⁷ is a residue of a saccharide.

12. The method of claim 11, wherein the residue of the saccharide is a monosaccharide.

13. The method of claim 12, wherein the monosaccharide is 2-deoxyribose, fructose, idose, gulose, talose, galactose, mannose, altrose, allose, xylose, lyxose, arabinose, ribose, threose, glucosamine, erythrose, or the pyranoside thereof.

14. The method of claim 12, wherein the monosaccharide is a glucopyranoside.

15. The method in any of claim 1, wherein R⁴ is a branched- or straight chain C₁ to C₂₅ alkyl group.

16. The method of claim 15, wherein R⁴ is a branched- or straight chain C₁ to C₁₀ alkyl group.

17. The method of claim 15, wherein R⁴ is a branched- or straight chain C₂ to C₉ alkyl group.

18. The method of claim 15, wherein R⁴ is a branched- or straight chain C₄ to C₉ alkyl group.

19. The method in any of claim 1, wherein R⁴ is methyl, ethyl, propyl, isopropyl, butyl, pentyl, hexyl, heptyl, octyl, nonyl, or octyl.

20. The method of claim 1, wherein R¹ is C(O)R⁸, wherein R⁸ is a branched- or straight chain C₁ to C₂₅ alkyl group.

21. The method of claim 1, wherein R¹ is C(O)NHR⁹, wherein R⁹ is a branched- or straight chain C₁ to C₂₅ alkyl group.

22. The method of claim 1, wherein R², R³, and R⁷ are hydrogen.

23. The method of claim 22, wherein R⁴ is a branched- or straight chain C₁ to C₂₅ alkyl group.

24. The method of claim 22, wherein R⁴ is methyl, ethyl, propyl, butyl, pentyl, or hexyl.

25. The method of claim 1, wherein the surfactant is the α-anomer.

26. The method of claim 1, wherein the surfactant is the β-anomer.

27. The method of claim 1, wherein the surfactant is an alkyl-β-D-thioglucopyranoside, an alkyl-β-D-thiomaltopyranoside, alkyl-β-D-galactopyranoside, an alkyl-β-D-thiogalactopyranoside, or an alkyl-β-D-maltrioside.

28. The method of claim 1, wherein the surfactant is hexyl-β-D-thioglucopyranoside, heptyl-β-D-thioglucopyranoside, octyl-β-D-thioglucopyranoside, nonyl-β-D-thioglucopyranoside, decyl-β-D-thioglucopyranoside, undecyl-β-D-thioglucopyranoside, dodecyl-β-D-thioglucopyranoside, octyl-β-D-thiomaltopyranoside, nonyl-β-D-thiomaltopyranoside, decyl-β-D-thiomaltopyranoside, undecyl-β-D-thiomaltopyranoside, or dodecyl-β-D-thiomaltopyranoside.

29. The method of claim 1, wherein the surfactant is an alkyl-β-D-glucopyranoside.

30. The method of claim 1, wherein the surfactant is hexyl-β-D-glucopyranoside, heptyl-β-D-glucopyranoside, octyl-β-D-glucopyranoside, nonyl-β-D-glucopyranoside, decyl-β-D-glucopyranoside, undecyl-β-D-glucopyranoside, dodecyl-β-D-glucopyranoside, tridecyl-β-D-glucopyranoside, tetradecyl-β-D-glucopyranoside, pentadecyl-β-D-glucopyranoside, hexadecyl-β-D-glucopyranoside, methyl-6-O—(N-heptylcarbamoyl)-α-D-glucopyranoside, 6-O-methyl-n-heptylcarboxyl)-α-D-glucopyranoside, or 3-cyclohexyl-1-propyl-β-D-glucopyranoside.

31. The method of claim 1, wherein the surfactant is an alkyl-β-D-maltopyranoside.

32. The method of claim 1, wherein the surfactant is 2-propyl-1-pentyl-β-D-maltopyranoside hexyl-β-D-maltopyranoside, heptyl-β-D-maltopyranoside, octyl-β-D-maltopyranoside, nonyl-β-D-maltopyranoside, decyl-β-D-maltopyranoside, undecyl-β-D-maltopyranoside, dodecyl-β-D-maltopyranoside, tridecyl-β-D-maltopyranoside, tetradecyl-β-D-maltopyranoside, pentadecyl-β-D-maltopyranoside, or hexadecyl-β-D-maltopyranoside.

33. The method of claim 1, wherein the surfactant is laetrile, arbutin, salicin, digitoxin, n-lauryl-beta-D-maltopyranoside, glycyrritin, p-nitrophenyl-beta-D-glucopyranoside, p-nitrophenyl-beta-D-galactopyranoside, p-nitrophenyl-beta-D-lactopyranoside, or p-nitrophenyl-beta-D-maltopyranoside.

34. The method of claim 1, wherein the surfactant is naturally-occurring.

35. The method of claim 34, wherein the surfactant is (Z)-5′-hydroxyjasmone 5′-O-beta-D-glucopyranoside, 3′-O-beta-D-glucopyranosyl-catalpol, prinsepiol-4-O-beta-D-glucopyranoside, fraxiresinol-4′-O-beta-D-glucopyranoside, quercetin 3-O-alpha-L-arabinopyranosyl-(1-->2)-beta-D-glucopyranoside, kaempferol 3-O-beta-D-glucopyranoside, quercetin 3-O-beta-D-glucopyranoside, catechin (4-alpha-->8) pelargonidin 3-O-beta-glucopyranoside, epicatechin (4-alpha-->8) pelargonidin 3-O-beta-glucopyranoside, afzelechin (4-alpha-->8) pelargonidin 3-O-beta-glucopyranoside, epiafzelechin (4-alpha-->8) pelargonidin 3-O-beta-glucopyranoside, quercetin 3,7-O-beta-D-diglucopyranoside, quercetin 3-O-alpha-L-rhamnopyransol-(1-->6)-beta-D-glucopyranosol-7-O-beta-D-glucopyranoside, isorhamnetin-3-O-beta-D-6′-acetylglucopyranoside, or isorhamnetin-3-O-beta-D-6′-acetylgalactopyranoside.

36. The method of claim 1, wherein the cells are undergoing sonoporation for compound delivery.

37. The method of claim 1, wherein the cells are tumor cells, or healthy cells in the vicinity of tumor cells, undergoing high intensity focused ultrasound (HIFU).

38. The method of claim 1, wherein the cells are healthy cells in the vicinity of a thrombus, undergoing high intensity focused ultrasound (HIFU).

39. The method of claim 1, wherein the cells are plant, animal or microbial cells in a bioreactor to which ultrasound is applied.

40. The method of claim 1, wherein the cells are brain cells during transcranial thrombolysis using focused ultrasound.

41. The method of claim 1, wherein the cells are corneal endothelial cells during phacoemulsification.

42. A method of protecting cells from ultrasound-mediated cytolysis comprising administering to the cells a surfactant, wherein the surfactant accumulates at the gas/liquid interface of cavitation bubbles, wherein the surfactant quenches a radical.

43. The method of claim 42, wherein the surfactant comprises a carbohydrate comprising at least one hydrophobic group.

44. The method of claim 42, wherein the carbohydrate is a monosaccharide.

45. The method of claim 42, wherein the monosaccharide is 2-deoxyribose, fructose, idose, gulose, talose, galactose, mannose, altrose, allose, xylose, lyxose, arabinose, ribose, threose, glucosamine, erythrose, or the pyranoside thereof.

46. The method of claim 42, wherein the monosaccharide is a glucopyranoside.

47. The method of claim 42, wherein the carbohydrate is a disaccharide.

48. The method of claim 42, wherein the disaccharide is lactose, cellobiose, or sucrose.

49. The method of claim 42, wherein the disaccharide is a maltosepyranoside.

50. The method of claim 42, wherein the carbohydrate is a polysaccharide.

51. The method of claim 42, wherein the polysaccharide is hyaluronan, chondroitin sulfate, dermatan, heparan, heparin, dermatan sulfate, and heparan sulfate, alginic acid, pectin, or carboxymethylcellulose.

52. The method of claim 42, wherein the hydrophobic group comprises a branched- or straight chain C₁ to C₂₅ alkyl group.

53. The method of claim 42, wherein hydrophobic group comprises a branched- or straight chain C₁ to C₁₀ alkyl group.

54. The method of claim 42, wherein hydrophobic group comprises a branched- or straight chain C₂ to C₉ alkyl group.

55-63. (canceled)

64. A method of protecting cells from ultrasound-mediated cytolysis comprising administering to the cells a surfactant wherein the surfactant is 3-cyclohexyl-1-propyl-β-D-glucoside.

65. A method of protecting cells from ultrasound-mediated cytolysis comprising administering to the cells a surfactant wherein the surfactant is 6-O-methyl-n-heptylcarboxyl-α-D-glucopyranoside.

66. A method of treating a tumor in a subject in need of such treatment, comprising:
a. administering to the area of the tumor an effective amount of a surfactant, wherein the surfactant accumulates at the gas/liquid interface of cavitation bubbles, wherein the surfactant quenches a radical; and
b. subjecting the tumor to high intensity focused ultrasound (HIFU), whereby the tumor is treated.

67. A method of delivering a compound to a cell comprising:
a. administering to the cells a composition comprising a surfactant wherein the surfactant accumulates at the gas/liquid interface of cavitation bubbles, wherein the surfactant quenches radicals; and
b. subjecting the cells to ultrasound frequencies sufficient to sonoporate the cells in the presence of the compound, thereby delivering the compound to the cells.

68-78. (canceled)

79. A composition comprising a surfactant in a suitable pharmaceutical carrier, wherein the surfactant can accumulate at the gas/liquid interface of cavitation bubbles wherein the surfactant can quench radicals.

80. A composition comprising a compound and at least one surfactant from claim 1 in a suitable pharmaceutical carrier, wherein the compound is delivered to cells in a subject by sonoporation.
